SEQUENCE LISTING (41 sequences)

SEQ ID NO: 1 - RNA, 70 nt, Homo sapiens
guuguuguaaacauccccgacuggaagcuguaagacacagcuaagcuuucagucagauguuugcugcuac

SEQ ID NO: 2 - RNA, 22 nt, Homo sapiens
uguaaacauccccgacuggaag

SEQ ID NO: 3 - RNA, 67 nt, Homo sapiens
uggagcugugugcagggccagcgcggagcccgagcagccgcggugaagcgccugugcucugccgaga

SEQ ID NO: 4 - RNA, 24 nt, Homo sapiens
ugaagcgccugugcucugccgaga

SEQ ID NO: 5 - RNA, 73 nt, Homo sapiens
uguauccuugaauggauuuuuggagcaggaguggacaccugacccaaaggaaaucaauccauaggcuagcaau

SEQ ID NO: 6 - RNA, 19 nt, Homo sapiens
aauggauuuuuggagcagg

SEQ ID NO: 7 - DNA, 19 bp, Artificial Sequence
  synthetic PCR Primer
tcctacgggaggcagcagt

SEQ ID NO: 8 - DNA, 26 bp, Artificial Sequence
  synthetic PCR Primer
ggactaccagggtatctaatcctgtt

SEQ ID NO: 9 - DNA, 23 bp, Artificial Sequence
  synthetic PCR Primer
cgtattaccgcggctgctggcac

SEQ ID NO: 10 - DNA, 21 bp, Artificial Sequence
  synthetic PCR Primer
cggtggagtatgtggcttaat

SEQ ID NO: 11 - DNA, 19 bp, Artificial Sequence
  synthetic PCR Primer
ccatgcagcacctgtgtaa

SEQ ID NO: 12 - DNA, 20 bp, Artificial Sequence
  synthetic PCR Primer
cgcctccgaagagtcgcatg

SEQ ID NO: 13 - DNA, 20 bp, Artificial Sequence
  synthetic PCR Primer
aggccttcgggttgtaaagt

SEQ ID NO: 14 - DNA, 20 bp, Artificial Sequence
  synthetic PCR Primer
cggggatttcacatctgact

SEQ ID NO: 15 - DNA, 20 bp, Artificial Sequence
  synthetic PCR Primer
cagaagaagcaccggctaac

SEQ ID NO: 16 - RNA, 22 nt, Artificial Sequence
  scrambled control sequence
guggaugaaccgcaacuaccau

SEQ ID NO: 17 - DNA, 20 bp, Artificial Sequence
  synthetic PCR Primer
ccatttacggcagaaacagc

SEQ ID NO: 18 - DNA, 20 bp, Artificial Sequence
  synthetic PCR Primer
gccagggagagggtttttac

SEQ ID NO: 19 - DNA, 20 bp, Artificial Sequence
  synthetic PCR Primer
cgtgaaggaaatagccctga

SEQ ID NO: 20 - DNA, 20 bp, Artificial Sequence
  synthetic PCR Primer
tgaaagggagggttcatctg

SEQ ID NO: 21 - DNA, 20 bp, Artificial Sequence
  synthetic PCR Primer
atccacacgggcagagtaat

SEQ ID NO: 22 - DNA, 20 bp, Artificial Sequence
  synthetic PCR Primer
tttatagaaatgcgggtggc

SEQ ID NO: 23 - DNA, 20 bp, Artificial Sequence
  synthetic PCR Primer
caacatggaaacctccatcc

SEQ ID NO: 24 - DNA, 20 bp, Artificial Sequence
  synthetic PCR Primer
gaccagttcctgggtgacat

SEQ ID NO: 25 - DNA, 20 bp, Artificial Sequence
  synthetic PCR Primer
agacttttgtggacatgggg

SEQ ID NO: 26 - DNA, 21 bp, Artificial Sequence
  synthetic PCR Primer
cggtggagtatgtggcttaat

SEQ ID NO: 27 - DNA, 19 bp, Artificial Sequence
  synthetic PCR Primer
ccatgcagcacctgtgtaa

SEQ ID NO: 28 - DNA, 20 bp, Artificial Sequence
  synthetic PCR Primer
cgcctccgaagagtcgcatg

SEQ ID NO: 29 - DNA, 34 bp, Artificial Sequence
  synthetic PCR Primer
gctctagagcatgaaatttgtcgccaaaatcctg

SEQ ID NO: 30 - DNA, 34 bp, Artificial Sequence
  synthetic PCR Primer
ggggtaccccttattcaatgctcttgagcacttc

SEQ ID NO: 31 - DNA, 47 bp, Artificial Sequence
  synthetic PCR Primer
gctctagagcatgaaatttgtcgcctaataatcctgaccatcgccgc

SEQ ID NO: 32 - DNA, 34 bp, Artificial Sequence; identical to SEQ ID NO: 30
  synthetic PCR Primer

SEQ ID NO: 33 - DNA, 40 bp, Artificial Sequence
  synthetic PCR Primer
gctctagagcatgaatgttatgtcgaaacgtttttttgcc

SEQ ID NO: 34 - DNA, 39 bp, Artificial Sequence
  synthetic PCR Primer
ggggtaccccatttaccgggtcagcatgccgttggctat

SEQ ID NO: 35 - DNA, 23 bp, Artificial Sequence
  synthetic PCR Primer
cccagtcacgacgttgtaaaacg

SEQ ID NO: 36 - DNA, 23 bp, Artificial Sequence
  synthetic PCR Primer
agcggataacaatttcacacagg

SEQ ID NO: 37 - RNA, 28 nt, Artificial Sequence
  miR-30d-5p binding sequence
cggcuccaacaucaccgacuggggcgau

SEQ ID NO: 38 - RNA, 33 nt, Artificial Sequence
  miR-30d-5p binding sequence
aucguuuauuucggggauguucagaaucugugg

SEQ ID NO: 39 - RNA, 40 nt, Artificial Sequence
  miR-30d-5p binding sequence
cccugaagaaagaccugaacauccccgucuggauccacac

SEQ ID NO: 40 - protein, 320 aa, Akkermansia muciniphila
MKFVAKILTIAAALSSLSMGANQVTVDSSIKPYAPTSGVSGNLNAVGSDTLNNLMTLWAEGFNKKYPSVKVGVEGKGSSTAPPALTAGTAQLAPMSRQMKREEIAAFEAKYGYKPTEIKVALDAVAFFVNKNNPIQALTLTQIDSIFSSTFKRGGSNITDWGDAGVPSMKGKAISIYGRNSASGTNGFVKEIALKKGDYKNSVKEQPGSSAVVQGISSDEQGIGYSGIGYVTSGVKTLSLAEKSGKAAVQPSYDNCINGTYPLSRYLLIYVNKKPGEPLDTLTREFIKFIVSKDGQEIVTKDGYYPIPAKVSAEVLKSIE

SEQ ID NO: 41 - protein, 674 aa, Ktedonobacter racemifer
MQQYFPNKILYGGDYNPEQWSEETWREDMRLMKLAHVNMVSINIFSWTLLEPEPHKYHFEQLDRIMDMLAEHGIYADLATATASPPTWMSRLYPSMLPVTQQGVRMSHGSRQHYCPNSPDYRRKAGALVEQIATRYAKHPALKMWHLNNEYGCHTGVCYCENCAAAFRKWLQERYQTLDKVNQAWGTTFWSQHYYEWEDVLPPRATPAQTNPTQTLDYWRFMNDSLRGCYELEEEILRRTTPSIPLTTNLMVAFKPVDVFDWAKHMDIVSFDMYPTPHDGAAQVAMPHDIMRGAKGGQPHIVMEMSPSQVNWQPQTPHKRPGQLRMHIMQSIARGANGALFFQWRQSMAGAEKYHSAVVSHEGSEQNRIFKQVAQAGAELAKLASQVANTRIHAKVALLMDWQSWWSSEYQPGPSDQLRYYEQILTYYKALYARNIAVDIVSPEAELGENLGKYKLVIAPLLHMIMPGVEQKLKGFVEQGGTFLTTFFSGIVDEHEHVIPGGYPGALRELLGIYVEEFDPLTPQMSNEVVIEEGELRGRYAATRWGELVHLKGAQALARFGQDYYAQQPAITEHSYGQGKAYYVATHPEQRLVDALIKQICAQAGVEPVLSTPEGVEVTLREGEHGEKFYFVLNQSKERQQITLSEGSYTSLLDEGKVQETITIEPFDVLVLKG

(19) United States
(12) Patent Application Publication
(10) Pub. No.: US 2021/0348163 A1
(43) Pub. Date: Nov. 11, 2021

(54) The Microbiome as a Target of MicroRNAs for the Treatment of Disease

(71) Applicant: The Brigham and Women's Hospital, Inc., Boston, MA (US)

(72) Inventors: Shirong Liu, Boston, MA (US); Howard Weiner, Brookline, MA (US)

(21) Appl. No.: 17/270,711

(22) PCT Filed: Aug. 23, 2019
(86) PCT No.: PCT/US2019/047954

§ 371 Date: Feb. 23, 2021

Related U.S. Application Data
(60) Provisional application No. 62/722,136, filed on Aug. 23, 2018.

(51) Int. Cl. C12N 15/113; A61K 31/713

(52) U.S. Cl. CPC C12N 15/113; A61K 31/713; C12N 2320/35; C12N 2310/32; C12N 2310/141

(57) ABSTRACT

Methods for treating subjects who have autoimmune diseases including multiple sclerosis. The methods include administering, e.g., orally, one or more micro RNAs, e.g., miR-30d, miR-7706, and miR-1246, or mimics thereof.

### CLAIM OF PRIORITY

This application claims the benefit of U.S. Provisional Application Ser. No. 62/722,136, filed on Aug. 23, 2018. The entire contents of the foregoing are incorporated herein by reference.

### TECHNICAL FIELD

This application relates, at least in part, to methods for treating subjects who have autoimmune diseases including multiple sclerosis. The methods include administering, e.g., orally, one or more micro RNAs, e.g., miR-30d, miR-7706, and miR-1246, or mimics thereof.

### BACKGROUND

Multiple sclerosis (MS) is an autoimmune disease directed against the central nervous system (CNS) myelin, and is associated with demyelination, oligodendrocyte loss, reactive gliosis, and axonal degeneration (Baecher-Allan et al., 2018). MS is a heterogeneous, multifactorial disease influenced by both genetic and environmental factors (Baecher-Allan et al., 2018). Pathologically, activated autoreactive CD4+ T cells in the periphery migrate to the CNS and initiate the MS process. Interferon gamma (IFNγ)-producing Th1 and interleukin-17 (IL-17)-secreting Th17 CD4+ T cells play a central role in the pathogenesis of MS (Baecher-Allan et al., 2018). These responses can be regulated in the periphery and/or in the CNS by regulatory cells such as FoxP3+ regulatory T cells (Tregs) (Lu and Rudensky, 2009).

### SUMMARY

As shown herein, administration of certain miRNAs, including miR-30d, increased the abundance of the gut commensal Akkermansia muciniphila (A. muciniphila), which in turn induced cytokines in dendritic cells that drove Treg differentiation and ameliorated symptoms in the experimental autoimmune encephalomyelitis (EAE) model of MS. In addition, administration of miR-7706 and miR-1246 was also shown to ameliorate EAE. These findings identify new avenues of therapeutic intervention.

Thus provided herein are methods for treating, reducing risk of development or progression of, or reducing symptoms of, an inflammatory condition in a subject. The method comprise administering a therapeutically effective amount of a nucleic acid comprising a sequence that is identical to a contiguous sequence of at least 12 nucleotides present in mature miR-30d, miR-7706, and/or miR-1246 microRNA, to a subject in need thereof. Also provided herein are nucleic acids comprising a sequence that is identical to a contiguous sequence of at least 12 nucleotides present in mature miR-30d, miR-7706, and/or miR-1246 microRNA for use in a method of treating, reducing risk of development or progression of, or reducing symptoms of, an inflammatory condition in a subject, the method comprising administering a therapeutically effective amount of a nucleic acid comprising a sequence that is identical to a contiguous sequence of at least 12 nucleotides present in mature miR-30d, miR-7706, and/or miR-1246 microRNA, to a subject in need thereof.

Also provided herein are methods for reducing interferon gamma (IFNγ)-producing Th1 and/or interleukin-17 (IL-17)-secreting Th17 CD4+ T cells, and/or increasing regulatory cells such as FoxP3+ regulatory T cells (Tregs), in the periphery and/or in the CNS in a subject. The methods comprise administering a therapeutically effective amount of a nucleic acid comprising a sequence that is identical to a contiguous sequence of at least 12 nucleotides present in mature miR-30d, miR-7706, and/or miR-1246 microRNA, to a subject in need thereof. Additionally provided herein are nucleic acids comprising a sequence that is identical to a contiguous sequence of at least 12 nucleotides present in mature miR-30d, miR-7706, and/or miR-1246 microRNA for use in a method of reducing interferon gamma (IFNγ)-producing Th1 and/or interleukin-17 (IL-17)-secreting Th17 CD4+ T cells, and/or increasing regulatory cells such as FoxP3+ regulatory T cells (Tregs), in the periphery and/or in the CNS in a subject.

In some embodiments, the nucleic acid is 12-24 nucleotides long.

In some embodiments, the nucleic acid is identical to a contiguous sequence of at least 13, 14, 15, 16, 17, 18, 19, 20, 21, or 22 nucleotides present in mature miR-30d, miR-7706, and/or miR-1246 microRNA.

In some embodiments, the nucleic acid is a mature miRNA or miRNA mimic selected from miR-30d, miR-7706, and/or miR-1246, e.g., a miRNA mimic thereof.

In some embodiments, the methods include administering miR-30d; miR-7706; miR-1246; miR-30d and miR-7706; miR-30d and miR-1246; miR-7706 and miR-1246; or miR-30d, miR-7706, and miR-1246.

Further provided herein are methods for of increasing relative abundance of Akkermansia muciniphila in the gut microbiome of a subject. The methods include administering a therapeutically effective amount of a nucleic acid comprising a sequence that is identical to a contiguous sequence of at least 12 nucleotides present in mature miR-30d to a subject in need thereof. Also provided herein are nucleic acids comprising a sequence that is identical to a contiguous sequence of at least 12 nucleotides present in mature miR-30d for use in a method of increasing relative abundance of Akkermansia muciniphila in the gut microbiome of a subject in need thereof.

In some embodiments, the nucleic acid is 12-24 nucleotides long. In some embodiments, the nucleic acid is identical to a contiguous sequence of at least 13, 14, 15, 16, 17, 18, 19, 20, 21, or 22 nucleotides present in mature miR-30d microRNA. In some embodiments, the nucleic acid is a mature miR-30d miRNA or miRNA mimic of miR-30d.

In some embodiments, the subject has an inflammatory condition. In some embodiments, the subject has cancer, e.g., a solid tumor, e.g., and is being treated with immunotherapy, e.g., a checkpoint inhibitor antibody.

In some embodiments, the condition is an inflammatory autoimmune disease.

In some embodiments, the condition is selected from the group consisting of Type 1 diabetes; multiple sclerosis; inflammatory bowel disease (IBD)/colitis; obesity and obesity-related conditions; epilepsy; immune-mediated liver injury; amyotrophic lateral sclerosis (ALS); rheumatoid arthritis; and aging or progeria.

In some embodiments, the nucleic acid is a miRNA mimic. In some embodiments, the miRNA mimic comprises one or more modifications. In some embodiments, the modifications include but are not limited to: double-stranded sequence, 5′ Amino-Modifier C6, and/or 3′ [dT][dT].

In some embodiments, the nucleic acid is administered orally or rectally.

In some embodiments, the nucleic acids are formulated to be administered orally or rectally.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D. Analysis of Gut Microbiome Changes During MOG-induced EAE.

(A-D) Mice were immunized with OVA or MOG and feces were collected at day 0 (0-day post immunization, 0 d.p.i., naïve), 8 d.p.i. (prior to EAE symptom onset for MOG-immunized mice), and 15 d.p.i. (peak EAE for MOG-immunized mice). (A-C) Bacterial 16S rDNA sequence-based microbiome surveys were performed. (A) Principal coordinates analysis (PCoA) based on unweighted UniFrac metrics. (B) Principal coordinates analysis (PCoA) based on weighted UniFrac metrics. (C) Relative abundance of bacteria classified at a species-level taxonomy. OVA n=5, MOG n=10. One-way ANOVA Dunnett's multiple comparisons test. Arrow highlights species that were increased at EAE peak; MOG 15 d.p.i. vs MOG 0 d.p.i. P=0.0001, MOG 15 d.p.i. vs MOG 8 d.p.i. P=0.0099, MOG 15 d.p.i. vs OVA 15 d.p.i. P=0.05. (D) qPCR quantification of the relative abundance of Akkermansia muciniphila (A. muciniphila) by measuring 16S rDNA, referenced to universal bacterial 16S rDNA. Naïve n=27, 15 d.p.i. of OVA n=19, 15 d.p.i. of MOG n=23. Error bars denote mean±SEM, One-way ANOVA Tukey's multiple comparisons test.

FIGS. 2A-I. Both Fecal Transfer and Fecal miRNA Transfer from Peak EAE Donor Ameliorate EAE in Recipients.

(A-B) The effect of transfer of feces from different stages of EAE on EAE in recipient mice. (A) Schematic of experimental design. Donor mice were immunized with MOG/CFA to induce EAE. Feces were collected at day 0 (naïve), day 8 post immunization (8 d.p.i., prior to symptom onset), and 15 d.p.i. (peak) and orally gavaged to recipient mice 6 days-, 4 days- and 2 days-prior to the induction of EAE in the recipients. (B) Clinical scores of EAE (left) and linear regression curves (right) in recipient mice. Combined data of two independent experiments at 0 d.p.i. (n=12), 8 d.p.i. (n=5), and 15 d.p.i. (n=12). Error bars denote mean±SEM; statistical analysis by two-way ANOVA and linear regression. (C-D), Analysis of the role of live bacteria in the EAE fecal transfer. (C) Experimental scheme. Donor mice were immunized with MOG or ovalbumin (OVA). Feces were collected at 15 d.p.i. (EAE peak), heat-inactivated or kept intact, and orally gavaged to recipient mice 6 days-, 4 days- and 2 days-prior to EAE induction in the recipients. (D) Clinical scores of EAE (left) and linear regression curves (right) in the recipient mice. Representative data of two independent experiments with n=7 each group; Error bars denote mean±SEM, statistical analysis by two-way ANOVA and linear regression. (E-F) Effect of oral administration of MOG-induced EAE peak fecal RNA on EAE. (E) Experimental scheme. Donor mice were immunized with MOG or OVA. Feces were collected at 15 d.p.i. when the MOG-immunized mice were at peak of EAE. Fecal RNA was isolated from donor feces and orally gavaged 6 days-, 4 days- and 2 days-prior to induction of EAE in the recipients. (F) Clinical scores of EAE (left) and linear regression curves (right) in the recipient mice. Representative data of two independent experiments with n=10 each group; Error bars denote mean±SEM, statistical analysis by two-way ANOVA and linear regression. (G-I) Therapeutic effect of oral administration of EAE peak fecal RNA on established EAE. Fecal RNA isolated from EAE peak or OVA-immunized mice was orally gavaged to EAE recipients at the dose of 10 μs RNA in 200 μl H₂O/mouse daily for 7 consecutive days starting when recipients had a disease score=1. (G) Clinical scores of EAE (left) and linear regression curves (right) in the recipient mice, representative data of three independent experiments, H₂O (vehicle) n=13, OVA-induced n=12, MOG-induced n=13; Error bars denote mean±SEM, statistical analysis by two-way ANOVA and linear regression. (H) Histopathological evaluation of demyelination with Luxol Fast Blue (LFB) and axonal loss with Bielschowsky's silver (Silver) staining of representative spinal cord sections from naïve mice and EAE mice treated with feces from H₂O (vehicle) or EAE peak EAE feces. Arrows denote demyelination (LFB) and axonal lost (Silver) in H₂O (vehicle) treated EAE, scale bars, 500 μm. (I) Quantification of demyelination and axonal loss based on LFB and Silver staining for individual mice. Representative data of three independent experiments with n=6 mice/group, Error bars denote mean±SEM, one-way ANOVA Dunnett's multiple comparisons test. n.s. not significant, * P<0.05, ** P<0.01, ***P<0.001, ****P<0.0001.

FIGS. 3A-D. MiR-30d Is Enriched in Feces from EAE Animals at Peak Disease and in Feces from Untreated MS Patients.

(A-B), RNA was isolated from feces of non-immunized (naïve) mice, mice immunized with OVA or mice immunized with MOG at 15 days post immunization (peak EAE). (A) Fold change of the changed miRNAs in top 25 abundant miRNAs by small RNA-Seq. Data were normalized to total reads. * P<0.05, **P<0.01, n=5 each group, Error bars denote mean±SEM, two-way ANOVA Dunnett's multiple comparisons test. (B) higher expression of miR-30d-5p in MOG-immunized EAE peak was verified by qPCR; Non-immunized n=9, OVA-immunized n=12, MOG-immunized n=12, Error bars denote mean±SEM, one-way ANOVA Dunnett's multiple comparisons test. (C-D), RNA was isolated from feces of non-treated relapsing-remitting MS patients and healthy controls (HC). (C) Fold change of the top 25 miRNAs by small RNA-Seq. Data were normalized to total reads. * P<0.05, ** P<0.01, n=10 each group; Error bars denote mean±SEM, Mann Whitney test. (D) higher expressions of miR-30d-5p, miR-7706 and miR-1246 in MS patients were verified by qPCR, n=12 each group, Error bars denote median ±95% CI, Mann Whitney test. n.s. not significant, * P<0.05, **P<0.01, *** P<0.001.

FIGS. 4A-I. Oral Administration of Synthetic miR-30d Ameliorates EAE in a Recipient Gut microbiome-dependent Manner.

(A-B) synthetic miR-30d, scramble control, or H₂O(vehicle) was orally gavaged to EAE recipients starting at disease onset (day 11, disease score=1) daily for 7 consecutive days. (A) Clinical scores of EAE (left) and linear regression curves (right) in the recipient mice. Representative data of two independent experiments; H₂O(vehicle) n=8, scramble n=13, miR-30d n=11, Error bars denote mean±SEM, statistical analysis by two-way ANOVA and linear regression. (B) Quantification of demyelination and axonal loss for individual mice. Data combined from two independent experiments with n=8 mice/group; Error bars denote mean±SEM, one-way ANOVA Dunnett's multiple comparisons test. (C-D) Mice were immunized with MOG and orally administered synthetic miR-30d or scramble control daily at a dose of 1000 pmol in 200 μl H₂O/mouse for 7 consecutive days. Foxp3+ T cells in the total CD4+ T cell population (C) and in the Vβ11+CD4+ T cell population (D) in the spleen were analyzed by FACS. Left panel: Representative FACS plots of Foxp3+CD4+ T cells; Right panel: % of CD4+ Foxp3+ T cells in individual animals (n=4 per group). Error bars denote mean±SEM; one-way ANOVA Tukey's multiple comparisons test. (E-G) Effect on EAE of transfer of fecal microbiome from synthetic miR-30d treated mice. Donor mice were immunized with MOG and orally treated with H₂O (vehicle), scrambled-miR-30d, or miR-30d for 7 consecutive days. Feces were collected and used to colonize mice that were pre-treated with antibiotics (ABX) for 7 days prior to colonization. Recipient mice were then induced for EAE. (E) Experimental scheme. (F) Clinical scores of EAE (left) and linear regression curves (right) in the recipient mice. Combined data of two experiments with H₂O (vehicle) n=19, scramble n=21, miR-30d n=24; Error bars denote mean±SEM, statistical analysis by two-way ANOVA and linear regression. (G) Quantification of demyelination and axonal loss. Values for individual mice are shown, combined from 2 independent experiments. n=19, scramble n=19, miR-30d n=20; Error bars denote mean±SEM, one-way ANOVA Dunnett's multiple comparisons test. (H-I) ABX abrogated therapeutic effect of oral miR-30d on EAE. Synthetic miR-30d, scramble control, or H₂O (vehicle) control was orally gavaged to EAE recipients starting at the onset of disease (day 11, disease score=1) at a dose of 250 pmol in 200 μl H₂O/mouse daily for 7 consecutive days. Mice were simultaneously gavaged with an antibiotics mixture (ABX). (H) Clinical scores of EAE (left) and linear regression curves (right., Combined data from two independent experiments, H₂O (vehicle) n=10, Scrambled miR-30d n=11, miR-30d n=11, Error bars denote mean±SEM, statistical analysis by two-way ANOVA and linear regression. (I) Quantification of demyelination and axonal loss for individual mice from two independent experiments (n=6 mice/group); Error bars denote mean±SEM, one-way ANOVA Dunnett's multiple comparisons test. * P<0.05, ** P<0.01, *** P<0.001.

FIGS. 5A-I. MiR-30d Enhances β-galactosidase of A. muciniphila and Expands A. muciniphila in vivo.

(A) A. muciniphila genes (AMUC_RS06985, AMUC_RS07700, AMUC_RS10850) were predicted to be targeted by miR-30d by sequence alignment. SEQ ID NOs:37, 2, 38, 2, 39 and 2 are shown. (B) A. muciniphila was grown in the presence of synthetic miR-30d, scrambled miR-30d or H₂O (vehicle). Transcripts of the predicted targeting genes at log phase were quantified by qPCR normalized to 16S rRNA. H₂O (vehicle) n=13, scrambled miR-30d n=15, miR-30d n=15, Error bars denote mean±SEM, one-way ANOVA Dunnett's multiple comparisons test. (C) Protein sequence alignment of AMUC_RS06985 of A. muciniphila (SEQ ID NO:40) and β-galactosidase of Ktedonobacter racemifer (K. racemifer)(Krac_10625) (SEQ ID NO:41). (D) AMUC_RS06985 of A. muciniphila or its truncated sequence was cloned into a β-galactosidase-deficient (lacZAM15) E. coli. The cloned E. coli colonies were grown on an X-gal-containing agar. (E) A. muciniphila was grown on BHI agar containing lactose and β-galactosidase activity indicator, X-gal and was treated with synthetic miR-30d or scrambled miR-30d. β-galactosidase activity was quantified according to the color change, n=5 each group, Error bars denote mean±SEM, paired t test. (F-I) The effect of oral administration of synthetic miR-30d on the gut microbiome. Mice were immunized with MOG and orally gavaged with 250 pmol synthetic miR-30d, scramble or H₂O (vehicle) for 7 days. Feces were collected at day 7 and bacterial 16S rDNA sequence-based microbiome surveys were performed. (F) Experimental scheme. (G) Principal coordinates analysis (PCoA) based on weighted UniFrac metrics and (H) Relative abundance of bacteria by 16S sequencing was classified at a species-level taxonomy. H₂O (vehicle) n=14, scramble n=13, miR-30d n=13. One-way ANOVA Dunnett's multiple comparisons test. Arrow identifies species that were significantly higher in miR-30d group compared to the other two groups. H₂O (vehicle) vs miR-30d P=0.0129, scramble vs miR-30d P=0.0087. (I) qPCR quantification of the relative abundance of A. muciniphila by measuring the 16S rDNA gene, referenced to universal 16S rDNA. H₂O (vehicle) n=14, scramble n=14, miR-30d n=13. Error bars denote mean±SEM, One-way ANOVA Tukey's multiple comparisons test.

FIGS. 6A-G A. muciniphila Promotes Tregs by Stimulating the Production of Treg-driving Cytokines in Dendritic Cells and Suppresses EAE.

(A-B) Effect of orally gavaged A. muciniphila on established EAE. Fresh cultured log phase Akkermanisa, E. coli, or Brain Heart Infusion culture medium (Medium) was orally administered to EAE recipients in 200 μl culture medium daily starting at the onset of disease (day 11, disease score=1) for 7 consecutive days. (A) Clinical scores of EAE (left) and linear regression curves (right) in the recipient mice. Combined data of 3 experiments. Medium n=23, E. coli n=27, A. mucimphila n=28, Error bars denote mean±SEM, statistical analysis by two-way ANOVA and linear regression. (B) Quantification of demyelination and axonal loss for individual mice. Combined data of three independent experiments (n=6 mice/group); Error bars denote mean±SEM, one-way ANOVA Dunnett's multiple comparisons test. (C-D) Freshly cultured logarithmic phase A. mucimphila, E. coli, or Medium was orally administered to MOG-immunized mice in 200 μl culture medium/mouse daily for 7 consecutive days. Foxp3+ T cells in the total CD4+ T cell population (C) and in the Vβ11+CD4+ T cell population (D) in the spleen were analyzed by FACS. Left panel: Representative FACS plots of Foxp3+CD4+ T cells; Right panel: % of CD4+ Foxp3 T cells in individual animals (n=4 per group). Error bars denote mean±SEM, One-way ANOVA Tukey's multiple comparisons test. n.s. not significant, * P<0.05, ** P<0.01. (E) Sorted naive CD4+ T cells from Foxp3-GFP reporter mice were induced toward Treg cell differentiation for 3 days in the presence of TGF-β plus IL-2 and in the presence of either A. mucimphila or E. coli. (F) CD11c+ dendritic cells were sorted from the mesenteric lymph nodes (MLN) of naïve mice and stimulated with A. mucimphila or E. coli. Sorted naive CD4+ T cells from Foxp3-GFP reporter mice were added 24 hours after and were induced toward Treg cell differentiation for 3 days in the presence of TGF-β and IL-2. (E-F) 72 h after Treg induction, live CD4+ cells were gated and determined for Foxp3+(GFP+) T cells. Left panel: Representative FACS plots of Foxp3+CD4+ T cells; Right panel: % of CD4+ Foxp3+ T cells in individual replicates. Data represent the mean±SEM, (E) n=4 and (F) n=9, one-way ANOVA Dunnett's multiple comparisons test. (G) CD11c+ dendritic cells were sorted from the MLN of naïve mice and stimulated with E. coli or A. mucimphila for 24 hours. RNA was isolated and quantified for Tgfb, Il6, and Il1b by qPCR. Data represent the mean±SEM, n=7, one-way ANOVA Dunnett's multiple comparisons test. n.s. not significant, * P<0.05, ** P<0.01, *** P<0.001, **** P<0.0001.

FIG. 7. Intestinal Dendritic Cells Are Responsible for the Generation of miR-30d Specifically upon MOG Immunization.

Mice were immunized with MOG or OVA/CFA. 10 days post immunization, dendritic cells, epithelial cells, macrophages, TCR αβ+ and TCR γδ+ intraepithelial lymphocytes (IEL) in the colon were sorted. The expression of miR-30d-5p in these cells was determined by qPCR. n=6, Error bars denote mean±SEM, one-way ANOVA Dunnett's multiple comparisons test. n.s. not significant, **P<0.01, *** P<0.001.

FIGS. 8A-B. The Dose Response of Oral Administration of Synthetic miR-30d in Ameliorating EAE.

The indicated dose of synthetic miR-30d, scrambled sequence control, or H₂O as blank control were orally administered to MOG/CFA-induced EAE mice starting from when the mice were scored 1, for 7 consecutive days. (A) Clinical scores of EAE (left) at the end of treatment (17 d.p.i) and 1 day post the end of treatment (18 d.p.i). Sample size of each group is indicated, Error bars denote mean±SEM, statistical analysis by two-way ANOVA. (B) Quantification of demyelination (LFB and MBP) and axonal loss (Silver and Neurofilament) for individual mice were determined by histological staining of the spinal cords. n=6 mice/group; Error bars denote mean±SEM, one-way ANOVA Dunnett's multiple comparisons test.

FIG. 9. Oral Administration of Synthetic miR-30d at the Dose of 250 pmol Increases Foxp3+ Regulatory T Cells.

Mice were immunized with MOG and orally administered synthetic miR-30d or scrambled miR-30d control daily at a dose of 250 pmol in 200 μl H₂O/mouse for 7 consecutive days. Foxp3+ T cells in the total CD4+ T cell population in the spleen were analyzed by FACS. Left panel: Representative FACS plots of Foxp3+CD4+ T cells; Right panel: % of CD4+ Foxp3+ T cells in individual animals (n=10 per group). Error bars denote mean±SEM; one-way ANOVA Tukey's multiple comparisons test. * P<0.05, *** P<0.001.

FIGS. 10A-B. Treg-Promoting Effect of Oral MiR-30d Administration is not Caused by Acting on T cell Differentiation Directly.

(A) Mice were immunized with MOG and orally administered synthetic miR-30d or scrambled control daily at a dose of 1000 pmol in 200 μl H₂O/mouse for 7 consecutive days. miR-30d level in the serum specimen were quantified by qPCR. n=5 per group, Error bars denote mean±SEM; one-way ANOVA Tukey's multiple comparisons test. n.s.<not significant. (B) Naïve CD4+ T cells from C57BL/6 spleen were differentiated into Treg (Foxp3+), Th17 (IL-17A+) and Th1 (IFN-γ+) cells by plate bound anti-CD3 and anti-CD28 in the presence of corresponding polarizing cytokines. The direct effect of miR-30d on T cell differentiation was examined by supplying synthetic miR-30d to the culture. T cell subsets were analyzed by FACS. Left panel: Representative FACS plots of T cell subsets; Right panel: Bar graph of % of T cell subsets individual culture. Error bars denote mean±SEM; one-way ANOVA Tukey's multiple comparisons test. * P<0.05, n.s.<not significant.

FIG. 11. Orally Administered miR-30d Keeps Intact to the Gut in Forms of Microvesicles and Non-vesicles.

Germ-free mice were orally administered synthetic miR-30d 1000 pmol in 200 μl. Fecal specimen were collected dynamically. Microvesicle (220 nm-800 nm) fractions, Exosome (20 nm-220 nm) fractions and Non-Vesicle (Vesicle-free, <20 nm) fractions of the feces were separated by size filtration. RNA was isolated and miR-30d, and as control, miR-1224 level in the fecal specimen were quantified by qPCR. n=2 per group, Error bars denote mean±SEM; one-way ANOVA Tukey's multiple comparisons test. **P<0.01, ***P<0.001.

FIG. 12. Detection of β-galactosidase (lactase) Activity in A. muciniphila which Hydrolyzes Lactose into Dextrose (glucose) that is Essential for A. muciniphila.

Scheme of β-galactosidase activity test with X-gal.

FIGS. 13A-B. MiR-30d enter A. muciniphila and Promote the Growth of A. muciniphila in vitro.

(A) A. muciniphila was cultured in presence of synthetic miR-30d or scrambled control for 18 hours to an exponential phase. miR-30d in A. muciniphila was determined by in situ hybridization using a 5′-DIG and 3′-DIG dual labeled probe for miR-30d and 10 nm immuno gold-conjugated anti-Digoxigenin antibody. (B) Synthetic miR-30d or scrambled control were supplied in a mixed culture of A. muciniphila and E. coli for 18 hours. The relative abundance of A. muciniphila and E. coli was determined by qPCR detecting 16S rDNAs of A. muciniphila and E. coli. n=8 per group, Error bars denote mean±SEM; one-way ANOVA Tukey's multiple comparisons test. ** P<0.01, n.s.<not significant.

FIGS. 14A-B. Oral Administration of Synthetic MiR-1246 and MiR-7706 Ameliorated EAE.

(A-B) synthetic miR-1246, miR-7706, scrambled miR-7706 control, or H2O (vehicle) was orally gavaged to EAE recipients at the dose of 250 pmol starting at disease onset (day 11, disease score=1) daily for 7 consecutive days. (A) Clinical scores of EAE (left) and linear regression curves (right) in the recipient mice. Sample size: H₂O n=8, Scramble n=5, miR-7706 n=11, miR-1246 n=11; Error bars denote mean±SEM, statistical analysis by two-way ANOVA and linear regression based on the scores from the start of the treatment (11 d.p.i) until the end of experiment. (B) Quantification of demyelination and axonal loss for individual mice. n=5 per group; Error bars denote mean±SEM, one-way ANOVA Dunnett's multiple comparisons test.

FIG. 15. Oral Administration of Synthetic MiR-30d Ameliorates Chronic Progressive EAE.

Chronic progressive EAE was induced in 8-week-old female NOD/ShiLtJ (Commonly called NOD) mice by subcutaneous immunization with 150 μg of MOG35-55 peptide in 4 mg/ml CFA. Pertussis toxin was given i.p. (150 ng per mouse) at the time of immunization and 48 h later. 250 pmol synthetic miR-30d or scrambled miR-30d control was orally administered daily beginning on day 43 post immunization when mice were scored 2, for 14 consecutive days. Clinical scores of EAE in the recipient mice were monitored. n=13 animals per group; Error bars denote mean±SEM, statistical analysis by two-way ANOVA.

FIGS. 16A-B. Oral Administration of Synthetic MiR-30d Reduces Type 1 Diabetes Incidence and Improves Hyperglycemia in NOD Mouse Model.

(A-B) NOD/ShiLtJ (commonly called NOD) mice spontaneously develop diabetic hyperglycemia starting at ˜12 weeks of age. 250 pmol of synthetic miR-30d or scrambled control were orally administered daily to the mice starting 8 weeks of age for 11 consecutive days. Blood glucose level (A) and diabetes incidence (B) were monitored once per week. n=10 animals per group; Error bars denote mean±SEM, statistical analysis by two-way ANOVA.

FIGS. 17A-B. Oral Administration of Synthetic MiR-30d Improves Type 2 Diabetes/Obesity in High Fat Diet Induced Obesity (DIO) Mouse Model.

(A-B) High fat diet (HFD) induced diabetes mice (C57BL/J DIO stock No: 380050; Black 6 DIO, the Jackson Laboratory) were kept on HFD (60 kcal % fat, 5.2 kcal/gram) and were orally gavaged synthesized miR-30d or scrambled control at the dose of 500 pmol every other day for 8 weeks starting at 12 weeks of age. intraperitoneal glucose tolerance test (IPGTT) was used to assess the ability of metabolizing glucose (A), and lipids (Cholesterol, Triglycerides) and Lactate dehydrogenase (LDH) in sera were measured (B) by the end of treatment.

### DETAILED DESCRIPTION

The gut microbiome plays an important role in the development of immune system (An et al., 2014; Belkaid and Hand, 2014; Hooper et al., 2012). Different commensals in the gut have been shown to promote the differentiation of subsets of lymphocytes. In mice, segmented filamentous bacteria induce intestinal Th17 cells (Ivanov et al., 2009), Bacteroides fragilis (B. fragilis) colonization of germ-free mice preferentially induces Th1 cells (Mazmanian et al., 2005), and polysaccharide A of B. fragilis suppresses Th17 cells in conventional mice by promoting IL-10 producing in Tregs through a TLR2 signaling pathway (Round et al., 2011). Clusters IV and XIVa of Clostridium promotes a transforming growth factor-β (TGF-β)-rich environment in the gut and Treg accumulation (Atarashi et al., 2011). The human symbiont Clostridium ramosum was also demonstrated to induce Treg (Sefik et al., 2015; Yissachar et al., 2017).

The gut microbiome has been linked to many disorders including inflammatory bowel disease (Ott et al., 2004), obesity (Turnbaugh et al., 2008), diabetes (Qin et al., 2012), and autism (Hsiao et al., 2013) and modulation of gut microbiome is being explored as a therapeutic modality. One such approach is fecal microbiome transplantation (FMT) for which there are more than 200 registered clinical trials (Schmidt et al., 2018). It is not clear whether FMT is a result of the transfer of microbes as the transfer of sterile filtrates from donor stool, rather than fecal microbes, was efficacious in patients with Clostridium difficile infection (Ott et al., 2017), raising the possibility that FMT may not act by microbial transfer but by transplantation of other fecal component(s) which in turn modulate the microbiome.

We and others have detected an altered gut microbiome in MS (Berer et al., 2017; Cekanaviciute et al., 2017; Chen et al., 2016; Jangi et al., 2016; Tremlett et al., 2016) and we have previously identified microRNAs (miRNA, miR) in the feces and found that fecal miRNA can shape the gut microbiome (Liu et al., 2016). In line with this, a recent study found that ginger-derived miRNAs can be taken up by the gut microbes, alter the microbial composition, and modulate the host physiology (Teng et al., 2018). Here, in order to investigate how the altered gut microbiome affects the course of MS, and whether fecal miRNA may be involved, we studied the gut microbiome and miRNA in the experimental autoimmune encephalomyelitis (EAE) model of MS. Unexpectedly, transfer of feces from EAE peak disease was protective when EAE was induced in recipient animals. We found that miR-30d, rather than live microbes, was responsible for the disease amelioration following fecal transfer. Furthermore, we found that miR-30d increased the abundance of the gut commensal Akkermansia muciniphila (A. muciniphila).

A. muciniphila is a mucin-degrading bacterium (Derrien et al., 2004) that has been reported to have anti-inflammatory properties. It has been shown that A. muciniphila improved diet-induced obesity (Everard et al., 2013) in a mechanism likely dependent on a specific protein (Amuc 1100) isolated from the outer membrane of A. muciniphila (Plovier et al., 2017). Furthermore, oral administration of A. muciniphila was shown to enhance glucose tolerance and attenuate adipose tissue inflammation by inducing Foxp3+ Tregs in the visceral adipose tissue (Shin et al., 2014). Of note, treatment with metformin increased A. muciniphila (Wu et al., 2017), and metformin treatment has been shown to attenuate EAE (Nath et al., 2009). Consistent with these studies, Hansen et al. reported that early life treatment with vancomycin propagated A. muciniphila and reduced diabetes incidence in the NOD mouse (Hansen et al., 2012). Furthermore, A. muciniphila has been shown to be associated with the anti-seizure effects of a ketogenic diet (Olson et al., 2018) and very recently shown to improve SOD1-Tg model of Amyotrophic Lateral Sclerosis (Blacher et al., 2019). Decreased A. muciniphila has been shown to be associated with progeria in humans and transplantation of A. muciniphila was sufficient to enhance healthspan and lifespan in progeroid mouse models (Barcena et al., 2019). Several groups have reported an increase of A. muciniphila in the gut microbiome of MS subjects (Berer et al., 2017; Cekanaviciute et al., 2017; Jangi et al., 2016; Tremlett et al., 2016). Cekanaviciute et al. found that A. muciniphila increased Th1 differentiation in vitro but found no effect in A. muciniphila-monocolonized mice (Cekanaviciute et al., 2017). In this study, we found that cell-mediated autoimmune diseases such as EAE in mice and MS in humans induced miR-30d upregulation in intestinal DCs and in the stool specimen. Although the mechanisms underlying this miRNA upregulation remains to be elucidated, the present results showed that oral administration of miR-30d expanded A. muciniphila in the EAE mouse gut by directly regulating gene expression of AMUC_RS06985, which we identified to be a new β-galactosidase in A. muciniphila. A. muciniphila in turn induced upregulation of TFG-β and downregulation of IL-6 and IL-10 transcripts by DCs in mesenteric lymph nodes (MLN), favoring Treg expansion that control effector T cells during EAE (Koutrolos et al., 2014).

Given that the microbiome plays an important role in health and disease (An et al., 2014; Fung et al., 2017; Honda and Littman, 2016; Hooper et al., 2012; Jangi et al., 2016; Qin et al., 2012; Tremaroli and Bäckhed, 2012), a major unmet need is to find approaches by which the microbiome can be specifically manipulated (Schmidt et al., 2018). FMT has been shown to be effective in the treatment of recurrent Clostridium difficile infection (van Nood et al., 2013), and is being investigated as a potential treatment for a number of disease conditions (Schmidt et al., 2018). Although promising, FMT is a complex biologic intervention without well-defined targets (Ianiro et al., 2014). More importantly, in practice, currently only feces from “Healthy” donor are used in most FMTs (Schmidt et al., 2018). While their effects on diseases have not been fully evaluated, feces from patients and diseased models have been excluded from FMT trials. As shown herein, feces from peak diseased donors improved the disease, and synthetic miRNAs were identified that can specifically modulate the microbiome and ameliorate inflammatory autoimmune disease. Of note, the miRNAs were identified in the feces of both EAE mice and untreated MS patients, which suggests that fecal miRNAs may represent a previously unrecognized process by which the host regulates the microbiome. These findings identify a new avenue for modulating the microbiome and raise the possibility that the feces of animals with disease and patients may be enriched for miRNAs with therapeutic properties.

Methods of Treatment

The present methods can be used to treat, risk of development or progression of, or reduce symptoms of, inflammatory conditions in a subject. As used in this context, to “treat” means to ameliorate at least one symptom of the disorder. The methods described herein include methods for the treatment of disorders associated with inflammation, e.g., as described herein. Generally, the methods include administering a therapeutically effective amount of one or more miRNAs as described herein, to a subject who is in need of, or who has been determined to be in need of, such treatment. The miRNAs can include, e.g., miR-30d, miR-7706, and/or miR-1246. The methods can include administering miR-30d; miR-7706; miR-1246; miR-30d and miR-7706; miR-30d and miR-1246; miR-7706 and miR-1246; or miR-30d, miR-7706, and miR-1246.

The conditions that can be treated include inflammatory autoimmune diseases in a subject. Inflammatory diseases include Type 1 diabetes; multiple sclerosis; inflammatory bowel disease (IBD)/colitis; obesity and obesity-related conditions; epilepsy; immune-mediated liver injury; amyotrophic lateral sclerosis (ALS); rheumatoid arthritis; and aging or progeria.

In some embodiments, the methods can be used to reduce interferon gamma (IFNγ)-producing Th1 and/or interleukin-17 (IL-17)-secreting Th17 CD4+ T cells, and/or increase regulatory cells such as FoxP3+ regulatory T cells (Tregs), in the periphery and/or in the CNS.

In some embodiments, the condition is one that has been shown to be improved by increasing Akkermansia muciniphila. For example, Akkermansia muciniphila has been shown to improve metabolism in obese and diabetic mice, and in overweight and obese human (see, e.g., Plovier et al., A purified membrane protein from Akkermansia muciniphila or the pasteurized bacterium improves metabolism in obese and diabetic mice. Nat Med. 2017; 23(1):107-113; Depommier et al., Supplementation with Akkermansia muciniphila in overweight and obese human volunteers: a proof-of-concept exploratory study. Nat Med. 2019; 25(7):1096-1103; Shin et al., An increase in the Akkermansia spp. population induced by metformin treatment improves glucose homeostasis in diet-induced obese mice. Gut. 2014; 63(5):727-35; and Everard et al, Cross-talk between Akkermansia muciniphila and intestinal epithelium controls diet-induced obesity. Proc Natl Acad Sci USA. 2013; 110(22):9066-71).

The present methods can be used to treat Type 1 diabetes, as it has been shown that vancomycin increases Akkermansia and reduces diabetes in NOD mouse. See, e.g., Hansen et al, Early life treatment with vancomycin propagates Akkermansia muciniphila and reduces diabetes incidence in the NOD mouse. Diabetologia. 2012; 55(8):2285-94.

Orally administered Akkermansia muciniphila has been shown to protect from immune-mediated liver injury in mouse model; see e.g., Wu et al., Protective Effect of Akkermansia muciniphila against Immune-Mediated Liver Injury in a Mouse Model. Front Microbiol. 2017; 8:1804.

The ketogenic diet (KD) has been used to treat refractory epilepsy. Akkermansia has been shown to mediate ketogenic diet protection of seizures, see Olson et al, The Gut Microbiota Mediates the Anti-Seizure Effects of the Ketogenic Diet. Cell. 2018; 173(7):1728-1741.e13.

Amyotrophic Lateral Sclerosis (ALS) is a genetically-driven neurodegenerative disorder. Akkermansia muciniphila has been shown to ameliorate mouse-ALS symptoms in a SOD1-Tg mice model, see Blacher et al, Potential roles of gut microbiome and metabolites in modulating ALS in mice. Nature. 2019; DOI: 10.1038/s41586-019-1443-5.

In addition, gut microbiota play an important part in the pathogenesis of mucosal inflammation, such as inflammatory bowel disease (IBD). Extracellular vesicles (EV) from Akkermansia muciniphila protected from DSS-induced IBD phenotypes, see Kang et al., Extracellular vesicles derived from gut microbiota, especially Akkermansia muciniphila, protect the progression of dextran sulfate sodium-induced colitis. PLoS One. 2013; 8(10):e76520.

Further, while the precise role of gut microbiome in aging has not been well elucidated, in two different mouse models of progeria Barcena et al found that progeria is characterized by intestinal dysbiosis with alterations in gut microbiome including a decrease in the abundance of Verrucomicrobia which Akkermansia belongs to. They found that human progeria patients also display intestinal dysbiosis and that long-lived humans (that is, centenarians) exhibit a substantial increase in Verrucomicrobia. Using the mouse models of progeria, they found that transplantation with the verrucomicrobia Akkermansia muciniphila was sufficient to enhance healthspan and lifespan in both progeroid mouse models. These findings provide a rationale for microbiome-, particularly Akkermansia-based interventions against age-related diseases. See Barcena, C. et al. Healthspan and lifespan extension by fecal microbiota transplantation into progeroid mice. Nat Med 25, 1234-1242 (2019).

Finally, Akkermansia muciniphila was shown to improve the efficacy of immunotherapy, e.g., anti-PD-1, in tumor therapy, and thus the present methods may be used in treating subjects with cancer, e.g., combination with immunotherapy in the treatment of cancers, e.g., solid tumors including. See Routy et al, Gut microbiome influences efficacy of PD-1-based immunotherapy against epithelial tumors. Science. 2018; 359(6371):91-97. Immunotherapy can include administration of an immunotherapy compound, e.g., an immune checkpoint inhibitory antibody, e.g., to PD-L1, PD-1, CTLA-4 (Cytotoxic T-Lymphocyte-Associated Protein-4; CD152); LAG-3 (Lymphocyte Activation Gene 3; CD223); TIM-3 (T-cell Immunoglobulin domain and Mucin domain 3; HAVCR2); TIGIT (T cell Immunoreceptor with Ig and ITIM domains); B7-H₃ (CD276); VSIR (V-set immunoregulatory receptor, aka VISTA, B7H5, C10orf54); BTLA 30 (B- and T Lymphocyte Attenuator, CD272); GARP (Glycoprotein A Repetitions; Predominant; 25 PVRIG (PVR related immunoglobulin domain containing); or VTCN1 (Vset domain containing T cell activation inhibitor 1, aka B7-H4). The methods can be used to treat a solid or hematopoietic tumor, e.g., melanoma, lung cancer (e.g., non-small cell lung cancer or small cell lung cancer), renal cell carcinoma, urothelial bladder cancer, hodgkins lymphoma, head and neck cancer, merkel cell carcinoma, MSI-H or dMMR cancer, colorectal cancer, gastic cancer, hepatocellular carcinoma, cervical cancer, PMBL, cutaneous squamous cell cancer, breast cancer, esophageal cancer, pancreatic cancer, ovarian cancer, and prostate cancer.

Pharmaceutical Compositions and Methods of Administration

The methods described herein include the use of pharmaceutical compositions comprising a miRNA described herein, e.g., human miR-30d, miR-7706, and/or miR-1246, as an active ingredient. The composition can include miR-30d; miR-7706; miR-1246; miR-30d and miR-7706; miR-30d and miR-1246; miR-7706 and miR-1246; or miR-30d, miR-7706, and miR-1246.

The human miR-30d precursor sequence is as follows: GUUGUUGUAAACAUCCCCGACUGGAAGCUGUAAGACACAGCUAAGCUU UCAGUCAGAUGUUUGCUGCUAC (SEQ ID NO:1), which has a predicted tertiary stem-loop structure as follows:

```
    guu u ccc gua ac
5′ gu guaaacauc gacuggaagcu ag a
          || ||||||||| ||||||||||| ||
3′ cg cguuuguag cugacuuucga uc c
    cau u --a --a ga
```

The mature hsa-miR-30d sequence is uguaaacauccccgacuggaag (SEQ ID NO:2).

The human miR-7706 precursor sequence is as follows: UGGAGCUGUGUGCAGGGCCAGCGCGGAGCCCGAGCAGCCGCGGUGAAG CGCCUGUGCUCUGCCGAGA (SEQ ID NO:3), which has a predicted tertiary stem-loop structure as follows:

```
    uggag g u - - gga - ag
5′ cu ug gcagggc cag cgc gcc cg c
              || || ||||||| ||| ||| ||| ||
3′ ga gc cgucucg guc gcg ugg gc a
    ----a - - u c aag c cg
```

The mature hsa-miR-7706 sequence is ugaagcgccugugcucugccgaga (SEQ ID NO:4).

The human miR-1246 precursor sequence is as follows: UGUAUCCUUGAAUGGAUUUUUGGAGCAGGAGUGGACACCUGACCCAAA GGAAAUCAAUCCAUAGGCUAGCAAU (SEQ ID NO:5), which has a predicted tertiary stem-loop structure as follows:

```
    -- -au uga ------- ag a u
5′ ugu ccu auggauu uuugg cagg g g
        ||| ||| ||||||| ||||| |||| |
3′ acg gga uaccuaa aaacc gucc c g
    ua auc --- cuaaagg ca a a
```

The mature hsa-miR-1246 sequence is aauggauuuuuggagcagg (SEQ ID NO:6).

In some embodiments, the present methods include the administration of at least one miRNA; the miRNAs used herein include pre-miRNA and mature miRNA, or a mimic thereof. “miRNA mimics” are chemically synthesized nucleic acid based molecules. microRNA mimics imitate the function of endogenous microRNAs in cells and can be designed as mature molecules, double-stranded molecules, or miRNA precursors (e.g., pri- or pre-microRNAs). MicroRNA mimics can be include synthetic and/or natural, modified and/or unmodified RNA, DNA, RNA-DNA hybrids or alternative nucleic acid chemistries as are generally known in the art.

A miRNA mimic as used herein can be a double stranded nucleic acid having a guide strand that has a nucleic acid sequence that is similar, or in some cases identical, to a guide strand of a naturally occurring mature miRNA. Naturally occurring miRNAs are processed from long nucleic acids having secondary structural properties (referred to as pri-miRNA and pre-miRNA) to produce naturally occurring mature miRNA. The mature miRNA is a double stranded molecule of about 22 (e.g., 20-24 or 21-23) nucleotides in length.

In some embodiments, the miRNA includes a sequence with at least 80% sequence identity to the full sequence of the endogenous human miRNA (i.e., SEQ ID NO:2, 4, or 6). In some embodiments, the miRNA includes a sequence with at least 90% sequence identity to at least 12, 13, 14, 15, 16, 17, 18, 19, 20, 21, or 22 consecutive nucleotides of SEQ ID NO: 2, 4, or 6. In some embodiments, the miRNA includes a sequence with at least 91%, 92%, 93%, 94%, 95%, 96%, 97%, 98% or 99% sequence identity to the full length og SEQ ID NO:2, 4, or 6. In some embodiments the sequence of the miRNA mimic may include the same bases, but the base of the mimic may be modified, i.e. hydrophobically modified. In other cases the mimic may include one or more different bases or nucleotides than the naturally occurring mature miRNA.

One having skill in the art armed with the sequences provided herein will be able, without undue experimentation, to identify further sequences. In some embodiments, an inhibitory nucleic acid contain a sequence that is identical to at least 5, 6, 7, 8, 9, 10, 11, 12, 13, 14, 15, 16, 17, 18, 19, 20, 21, 22, 23, 24, or 25 continguous nucleotides present in the miRNA, e.g., mature or precursor miRNA). In some embodiments, the miRNAs comprise a sequence that is complementary to a contiguous sequence of at least, e.g, 13, 14, 15, 16, 17, 18, 19, 20, 21, 22, 23, 24, or 25, nucleotides present in a mature microRNA miR-30d, miR-7706, and/or miR-1246, e.g., having the sequence of SEQ ID NO:2, 4, or 6, optionally wherein the nucleic acid comprises at least one modified base.

miRNA mimics are available and known in the art. miRNA mimics can be, e.g., double-stranded RNA molecules, e.g., with at least one strand with at least 90% sequence identity to SEQ ID NO: 2, 4, or 6. A miRNA mimic can include one or more modifications, on one strand or on both sense and anti-sense strand, as compared to an endogenous (natural) miRNA, such as natural residues or non-natural residues substituted at one or more positions with respect to the endogenous miRNA sequence. Examples of nucleotides that can be employed in miRNA mimics can include, without limitation, 5-Amino-Modifier C6, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninj e, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-dT, 3-dTdT, 3-β-amino-3-N-2-carboxypropyl) uracil, (acp3)w, and 2,6-diaminopurine.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, lipids, lipidsome, nanoparticles, microvesicles, compatible with pharmaceutical administration. Supplementary active compounds can also be incorporated into the compositions. For example, for treating cancer the composition can include an immunotherapy compound, e.g., an immune checkpoint inhibitory antibody, e.g., to PD-L1, PD-1, CTLA-4 (Cytotoxic T-Lymphocyte-Associated Protein-4; CD152); LAG-3 (Lymphocyte Activation Gene 3; CD223); TIM-3 (T-cell Immunoglobulin domain and Mucin domain 3; HAVCR2); TIGIT (T cell Immunoreceptor with Ig and ITIM domains); B7-H3 (CD276); VSIR (V-set immunoregulatory receptor, aka VISTA, B7H5, C10orf54); BTLA 30 (B- and T Lymphocyte Attenuator, CD272); GARP (Glycoprotein A Repetitions; Predominant; 25 PVRIG (PVR related immunoglobulin domain containing); or VTCN1 (Vset domain containing T cell activation inhibitor 1, aka B7-H4). The supplementary active compounds can also be administered separately, e.g., as a combination therapy, e.g., in some embodiments the two compounds are administered concurrently (either in a single or separate compositions) or sequentially.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In the present methods, oral administration is preferred. Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The pharmaceutical compositions can also be prepared in the form of suppositories (e.g., with conventional suppository bases such as cocoa butter and other glycerides) or retention enemas for rectal delivery.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

Experimental Procedures

The following materials and methods were used in the Examples set forth below, unless otherwise noted.

Mice and Fecal Specimen Collection

Animal procedures were approved by the Harvard Medical Area (HMA) Standing Committee on Animals. C57BL/6J mice and Foxp3^{GFP+} mice (Stock No. 023800) were from The Jackson Laboratory and acclimated in the local animal facility for at least two weeks prior to study initiation. Otherwise specified, all mice used were 6-8 weeks old at the initiation of study. For all experiments (fecal transplantation, fecal RNA transplantation, synthetic microRNA administration, bacteria administration), mice of same age and gender were ear-tagged and randomly allocated into groups and co-housed. Mice were housed under specific pathogen-free conditions at the Harvard Institutes of Medicine and the Hale Building for Transformative Medicine at Brigham and Women's Hospital. Mice that received oral administration of Akkermansia muciniphila (A. muciniphila) or E. coli were housed in BSL-2N facility. Fecal specimens were collected immediately upon defecation, snap frozen, and stored at −80° C. for analysis.

Human Fecal Specimens

Human fecal specimens were collected from 12 healthy volunteers (9 females, average 49 years of age) and 12 untreated relapsing-remitting multiple sclerosis patients (11 females, average 47 years of age). All subjects gave written consent according to a protocol approved by the Institutional Review Board at Brigham and Women's Hospital. All subjects were excluded for GI disorders, antibiotics, or probiotic use in the last 2 months and during the sampling period. All stool samples were collected using Commode specimen collection system (Fisher Scientific) and stored at −80° C. until further processing.

EAE Induction

EAE was induced by injecting 6- to 8-week-old female C57BL/6J mice with 150 μg MOG35-55 peptide (Genemed Synthesis) emulsified in complete Freund's adjuvant (CFA)(BD™ Difco™) per mouse subcutaneously in the flanks, followed by intraperitoneal administration of 150 ng pertussis toxin (List biological laboratories, Inc.) per mouse on days 0 and 2 as described (Mayo et al., 2014). In some experiments, as an immunization control, 150 μg OVA₃₂₇₋₃₃₃ (Anaspec) were used to replace MOG₃₅₋₅₅. Clinical signs of EAE were assessed according to the following score: 0, no signs of disease; 1, loss of tone in the tail; 2, hind limb paresis; 3, hind limb paralysis; 4, tetraplegia; 5, moribund.

Histopathology

Mice were euthanized at the termination of experiments and were intracardially perfused with PBS, followed by fixation with Bouin's Fixative solution (RICCA Chemical). Tissue was processed and stained as previously described (Mayo et al., 2014). Paraffin embedded serial sections were stained with Luxol Fast Blue for myelin, Bielschowsky silver for axons. In dose response experiments, additional evaluation of demyelination and neuron loss was carried out using rabbit anti-MBP (1:1000; Dako) and neurofilament (1:3000; Abcam) respectively and with secondary biotinylated antibodies. Avidin-peroxidase and 3,4-Diaminobenzidine was used as the color substrate (Reuter et al., 2015). The demyelinated area and axonal/neuronal loss were determined using ImageJ software (National Institutes of Health, USA) and the percentages of demyelinated and axonal/neuronal lost area out of total area were calculated.

Fecal RNA Isolation

Total RNA (including miRNA) was extracted from stool specimens using mirVana™ miRNA isolation kit (catalog number: AM1560, Ambion®) following the established protocol (Liu et al., 2016). Briefly, mouse or human stool was homogenized in sterile PBS. RNA was extracted with acid Acid-Phenol: Chloroform. Aqueous phase precipitation was performed by mixing with 1.25 volumes of 100% ethanol, followed by purification on a glass fiber filter cartridge. Following elution, RNA quality was assessed by A260/A280 ratios using ND-1000 Nanodrop and Agilent 2100 Bioanalyzer (Agilent Technologies). The purity of RNA was A260/A280: A260/A230: ≥1.3. RNA isolates were stored at −80° C. until use.

Fecal Transplantation, Fecal RNA Treatment

For mouse fecal transplantation, 5 mg per mouse of feces from donor mice was suspended in 200 μl sterile PBS and was administered to recipient C57BL/6J mice by orally gavage at the time showed in the figures. In some cases, feces were inactivated by heating at 80° C. for 60 min to kill bacteria while keeping miRNA in the feces (Jung et al., 2010). To investigate the effect of fecal RNA on EAE, 10 μg of RNA isolated from feces, as described above, was eluted in 200 μl nuclease-free water and administered to mouse by orally gavage at the time as indicated in the figures.

Small RNA Sequencing

Small RNA-seq libraries were constructed from fecal RNA isolates using NEXTflex™ Small RNA-Seq Kit (Bioo Scientific Corporation., USA). 500 ng of RNA was used as input material. The library was prepared with a unique indexed primer so that libraries could be pooled into one sequencing flow cell. Multiplex adaptor ligations, primer hybridization, reverse transcription reaction and PCR amplification were performed according to the manufacturer's protocol. Libraries were further purified with a gel size selection using Blue Pippin (Sage Science, Inc. USA). The obtained libraries were checked for quality with Agilent 2200 TapeStation and were sequenced with the Illumina NextSeq 500 System (50 nt, single read) at the Biopolymers Facility at Harvard Medical School. Data were analyzed following the exceRpt small RNA-seq pipeline V4.6.2 (Subramanian et al., 2015). Normalization and differential expression were performed with the R package DESeq2 v.1.10.1 (R version 3.3.2) (Love et al., 2014).

MiRNA Measurement by qPCR

Quantitative PCR (qPCR) was performed to verify the relative level of miRNAs that were identified in small RNA-Seq. 200 ng of total fecal RNA was input for miRNA cDNA synthesis using TaqMan™ Advanced miRNA cDNA Synthesis kit (Applied Biosystems). MiRNA cDNAs were then quantified by real-time PCR using TaqMan® Fast Advanced Master Mix and TaqMan Advanced MiRNA Assays (Applied Biosystems) on QuantStudio™ 7 Flex Real-Time PCR System (Applied Biosystems) following the manufacturer's protocol: hsa-miR-21-5p/mmu-miR-21a-5p (Assay ID: mmu482709_mir), mmu-miR-30d-5p/hsa-miR-30d-5p (Assay ID: mmu478606_mir), hsa-miR-7706 (Assay ID: 480578_mir), hsa-miR-1246 (Assay ID: 477881_mir). A reference gene for quantifying fecal miRNA using qPCR has not been established. MiR-21 has been detected in both mouse and human feces (Johnston et al., 2018; Link et al., 2010; Liu et al., 2016; Schönauen et al., 2018). Our small RNA-seq data suggested that miR-21 was highly presented in mouse and human feces and was not distinguishable between healthy and MS patient and between naïve and immunized mice. We thus used miR-21 as reference to measure the relative level of miR-30d, miR-1246 and miR-7706 using comparative CT method (Schmittgen and Livak, 2008).

Antibiotic Treatment

In order to investigate the involvement of gut microbiome in the effect of miRNA, to deplete bacteria, mice were given a mixture of antibiotics (ampicillin 1 mg/ml, vancomycin 0.5 mg/ml, neomycin 1 mg/ml, metronidazole 1 mg/ml, and streptomycin 1 mg/ml (Sigma-Aldrich)), following an established protocol (Benjamin et al., 2013) in drinking water or in 200 μl nuclease-free water by orally gavage as specified on figure legends, for 7 consecutive days. Bacteria depletion was confirmed by culturing the colonic luminal content anaerobically on BHI agar and aerobically on LB agar.

16S rDNA Analyses of Gut Microbiome

16S rDNA sequence survey was performed following our established procedure (Liu et al., 2016; Tankou et al., 2018). Briefly, DNA in the mouse feces was extracted using a QIAamp Fast DNA Stool Mini Kit (Qiagen). Amplicons spanning variable region 4 (V4) of the bacterial 16S rRNA gene were generated with primers containing barcodes (515F, 806R) from the Earth Microbiome project (Caporaso et al., 2012) using HotMaster Taq and HotMaster Mix (QuantaBio) and paired-end sequenced on an Illumina MiSeq platform at the Harvard Medical School Biopolymer Facility. Data was processed using the QIIME 2 software following an established protocol (Caporaso et al., 2012; 2010). Briefly, sequences were de-multiplexed and quality filtered in which reads were truncated if two consecutive bases fall below a quality score of Q20 (1% error), and reads that were <75% of full length were discarded (Caporaso et al., 2012). OTUs were picked using the open reference method sumaclust (metabarcoding.org/sumatra) and sortmeRNA (Kopylova et al., 2012). Taxonomy was picked against the Greengenes database (greengenes.secondgenome.com) using a 97% similarity threshold.

Fecal Microbe Quantification by qPCR

DNA extracted from fecal pellets as above described was verified for specific bacteria abundance. Quantitative PCR (qPCR) analysis was conducted using a ViiA7 system (Applied Biosystems). A. muciniphila was quantified by Taqman amplification reactions consisting of DNA, TaqMan® Universal PCR Master Mix (Applied Biosystems), and primer pairs as follows: All bacteria (universal 16S rDNA, reference): Forward: TCCTACGGGAGGCAGCAGT (SEQ ID NO:7), Reverse: GGACTACCAGGGTATCTAATCCTGTT (SEQ ID NO:8), Probe: CGTATTACCGCGGCTGCTGGCAC (SEQ ID NO:9) (Nadkarni et al., 2002); A. muciniphila 16S rRNA gene: Forward: CGGTGGAGTATGTGGCTTAAT (SEQ ID NO:10), Reverse: CCATGCAGCACCTGTGTAA (SEQ ID NO:11), probe: CGCCTCCGAAGAGTCGCATG (SEQ ID NO:12). In some experiment, E. coli 16S rRNA gene was detected using the primers and probe: Forward: AGGCCTTCGGGTTGTAAAGT (SEQ ID NO:13), Reverse: CGGGGATTTCACATCTGACT (SEQ ID NO:14), Probe: CAGAAGAAGCACCGGCTAAC (SEQ ID NO:15). The relative quantity was calculated using the comparative CT method normalizing to the amount of all bacteria in the sample (Schmittgen and Livak, 2008).

MiRNA Target Prediction

The sequence of miR-30d-5p (uguaaacauccccgacuggaag (SEQ ID NO:2)) was blasted against whole genome sequence of A. muciniphila using the NCBI blast tool for sequence pairing. RNAhybrid was used to characterize the minimum free energy of secondary structure binding between miR-30d and potential targeting A. muciniphila RNA (Krüger and Rehmsmeier, 2006; Rehmsmeier et al., 2004).

Synthetic MiRNA Treatment

Synthesized Mission® miRNA mimics (Sigma-Aldrich) of miR-30d-5p (uguaaacauccccgacuggaag (SEQ ID NO:2)) and scrambled miR-30d-5p sequence miRNA control (guggaugaaccgcaacuaccau (SEQ ID NO:16)) were orally gavaged to C57BL/6J mice at a dose of 250 pmol daily in 200 μl nuclease-free water for 7 consecutive days.

The sequences of Mission® miRNA mimics used were (5′ to 3′): miR-30d-5p_antisense: [AmC6]CUUCCAGUCGGGGAUGUUUUACA[dT][dT] (SEQ ID NO:2); miR-30d-5p_sense: UGUAAACAUCCCCGACUGGAAG[dT][dT] (SEQ ID NO:2); hsa-miR-1246_antisense: [AmC6]CCUGCUCCAAAAAUCCUAUU[dT][dT] (SEQ ID NO:6); hsa-miR-1246_sense: aauggauuuuuggagcagg[dT][dT] (SEQ ID NO:6); hsa-miR-7706_antisense: [AmC6]UCUCGGCAGAGCACAGGCGCUUUCA[dT][dT] (SEQ ID NO:4); hsa-miR-7706 sense: ugaagcgccugugcucugccgaga[dT][dT] (SEQ ID NO:4). Feces were collected 24 hours post last synthetic miRNA administration for bacteria abundance detection. In dose response experiments (FIGS. 8A-B), different doses of miRNAs were used as indicated.

Dynamic miR-30d Measurement in the Gut (Feces) Post Oral Administration of miR-30d

Synthetic miR-30d was orally administered to germ-free mice and fecal specimen were collected at the gavage (0 hour), and every 2 hours post administration. Fecal sample was soaked in 2 ml cold PBS for 5 min, and dissociated with PowerLyzer®24 Homgenizer (Mo Bio Laboratories, CA). The suspension was centrifuged at 300×g, 4° C. for 10 min, followed by the additional centrifugation at 2000×g, 4° C. for 15 min. The supernatant was collected and filtered through a 0.8 μm filter (EMD Millipore, MA) to further remove debris. Microvesicle (MV), exosome and non-vesicle fractions were sequentially separated from the filtrate with 0.22 μm filter (EMD Millipore), 0.02 μm filter (GE Healthcare) and 3 kDa Amicon Ultra Centrifugal Filters (EMD Millipore), as previously described (Wei et al., 2017). Total RNA was isolated from each fraction using Total RNA Purification Kit (Norgen Biotek, Canada). The RNA concentrations were determined using Quant-iT RiboGreen RNA Assay Kit (Thermo Fisher Scientific). Two nanogram of total RNA was used in 10 μl reverse transcription reaction with Universal cDNA Synthesis kit II (Exiqon). The qPCR reaction was performed using the ExiLENT SYBR Green master mix and pre-designed LNA primers (Exiqon) and miR-21 as reference.

In experiment of determine miRNAs in serum, serum RNA was isolated using Plasma/Serum RNA Purification Kit (Norgen Biotek Corporation). The levels of miRNA were quantified with qPCR using the approach described in this section.

Bacteria Strains, Growth Conditions and Bacteria Administration

Akkermansia muciniphila Derrien et al. (ATCC® BAA-835™) and E. coli K-12 (Strain #: 7296, The Coli Genetic Stock Center at Yale) were grown anaerobically in Brain Heart Infusion (BHI) medium (SKU 53286, Sigma Aldrich). For mice treatment, 5×10⁸ freshly cultured logarithmic phase bacteria in 200 μl BHI medium were given by oral gavage daily for 7 consecutive days. For in vitro stimulation of cultured cells (FIG. 6E-G), A. muciniphila and E. coli K-12 were cultured to a logarithmic phase and harvested by spinning down at 12600 rpm. Bacteria were resuspended in sterile PBS to a density of OD600=1. The bacteria suspensions were inactivated by eight cycles of freezing at −80° C. and thawing at 37° C. The inactivation was confirmed by culturing the suspension to find no growing clone.

In experiments testing β-galactosidase activity in A. muciniphila, freshly cultured logarithmic phase A. muciniphila was spread or streaked over the surface of agar (1.5%) containing one of the following broth: BHI (with 0.2% dextrose), BHI w/o Dextrose (Ordering code: B2701-09, United States Biological), BHI w/o Dextrose plus 0.2% lactose (Catalog No. L6-500, Fisher Scientific), BHI w/o Dextrose plus 0.2% sucrose (SKU S0389, Sigma Aldrich), BHI w/o Dextrose plus 0.2% mucin from porcine stomach (SKU M1778, Sigma Aldrich), or BHI w/o Dextrose plus 0.2% mucin from porcine stomach and plus 400 μg/ml X-Gal (Catalog No. X4281, Gold Biotechnology), and incubated at 37° C. anaerobically for 5 days.

In experiments investigating the effect of miR-30d on A. muciniphila β-galactosidase (FIG. 5E), 10 μl of freshly cultured logarithmic phase A. muciniphila was inoculated to a Ø10 mm sterile disk (Item ID: 74146, Millipore Sigma) on BHI w/o Dextrose plus 0.2% lactose and plus 400 μg/ml X-Gal, and incubated at 37° C. anaerobically for 5 days, during which 30 μl of 100 μM synthetic miR-30d, or scrambled miR-30d was added at 24 h and 48 h after inoculation. β-galactosidase activity was quantified by measuring the color changed (blue) area around the disk with A. muciniphila using ImageJ.

Bacterial Gene Transcript Quantification by qPCR

A. muciniphila was cultured in the presence of H₂O (vehicle), 3 μM miRNA mimics miR-30d, and scrambled miR-30d to a log phase and stopped by chilling on ice and stabilized with RNAlater® Solutions (Ambion). Total bacterial RNA from cultured bacterial was extracted using TRIzol® Max™ Bacterial RNA isolation Kit (Ambion) following the manufacturer's protocol. cDNA was prepared using High Capacity cDNA Reverse Transcription Kit (Applied biosystems). QPCR was performed using Taqman Universal PCR Master Mix and TaqMan® Gene Expression Assay primer pairs as following: A. muciniphila AMUC_RS06985: Forward: CCATTTACGGCAGAAACAGC (SEQ ID NO:17), Reverse: GCCAGGGAGAGGGTTTTTAC (SEQ ID NO:18), probe: CGTGAAGGAAATAGCCCTGA (SEQ ID NO:19). A. muciniphila AMUC_RS07700: Forward: TGAAAGGGAGGGTTCATCTG (SEQ ID NO:20), Reverse: ATCCACACGGGCAGAGTAAT (SEQ ID NO:21), probe: TTTATAGAAATGCGGGTGGC (SEQ ID NO:22). A. muciniphila AMUC_RS10850: Forward: CAACATGGAAACCTCCATCC (SEQ ID NO:23), Reverse: GACCAGTTCCTGGGTGACAT (SEQ ID NO:X24X), probe: AGACTTTTGTGGACATGGGG (SEQ ID NO:25). The relative quantity of each bacterial gene transcripts was calculated by the ΔCt method and referenced to A. muciniphila 16S rRNA: Forward: CGGTGGAGTATGTGGCTTAAT (SEQ ID NO:26), Reverse: CCATGCAGCACCTGTGTAA (SEQ ID NO:27), probe: CGCCTCCGAAGAGTCGCATG (SEQ ID NO:28).

Construction of E. coli Strains Expressing AMUC_RS06985 and Detection of β-Galactosidase (Lactase) Activity

The genes AMUC_RS06985, truncated AMUC_RS06985, and AMUC_RS07700 were amplified by PCR using the following primers (with restriction sequences underlined): AMUC_RS06985 (XbaIAMUC_RS06985Fwd: 5′-GCTCTAGAGCATGAAATTTGTCGCCAAAATCCTG-3′ (SEQ ID NO:29), KpnIAUMC_RS06985Rev: 5′-GGGGTACCCCTTATTCAATGCTCTTGAGCACTTC-3′ (SEQ ID NO:30)), truncated AMUC_RS06985 (XbaITruncatedAMUC_RS06985Fwd:5′-GCTCTAGAGCATGAAATTTGTCGCCTAATAATCCTGACCATCGCCGC-3′ (SEQ ID NO:31), KpnITruncatedAUMC_RS06985Rev:5′-GGGGTACCCCTTATTCAATGCTCTTGAGCACTTC-3′ (SEQ ID NO:32)), and AMUC_RS07700 (XbaIAMUC_RS07700Fwd:5′-GCTCTAGAGCATGAATGTTATGTCGAAACGTTTTTTTGCC-3′ (SEQ ID NO:33), KpnIAUMC_RS07700Rev:5′-GGGGTACCCCATTTACCGGGTCAGCATGCCGTTGGCTAT-3′ (SEQ ID NO:34)).

PCR products of the genes containing XbaI and KpnI restriction sites were cloned into pUC18 (a gift from Joachim Messing, Addgene plasmid #50004) (Norrander et al., 1983) between the XbaI and KpnI sites of the vector. TOP10 competent E. coli cells (Genotype: F-mcrA Δ(mrr-hsdRMS-mcrBC) Φ80lacZ/IM15 lacX74 recA1 araD139 Δ(araleu)7697 galU galK rpsL (StrR) endA1 nupG) (Invitrogen) were transformed with constructed plasmids by heat shock. Cells with ampicillin resistance were selected by plating the transformed cells on LB agar containing 100 μg/ml Ampicillin. E. coli strains expressing the intended inserts were confirmed by sequencing using primers: M13pUC-Fwd 5′-CCCAGTCACGACGTTGTAAAACG-3′ (SEQ ID NO:35) and M13pUC-Rev 5′-AGCGGATAACAATTTCACACAGG-3′ (SEQ ID NO:36). To detect β-galactosidase activity of the constructed strains, bacteria were streaked on LB agar containing 100 μg/ml ampicillin and 400 μg/ml X-gal, and grew at 37° C. for 3 days.

Protein Sequence Alignment

The sequence of protein product of A. mucimphila gene AMUC_RS06985 (Accession ID: WP_012420345) was aligned to sequences of beta-galactosidases of different species available at UniProt (uniprot.org) using Protein BLAST tool from NCBI. Typical positive blast hit, the beta-galactosidase of Ktedonobacter racemifer DSM 44963 (Accession ID: EFH89096) (E value: 0.023) was further aligned using T-Coffee (Notredame et al., 2000) and viewed with Jalview (Waterhouse et al., 2009).

Flow Cytometry and Cell Isolation

To investigate the effect of miRNA or bacteria on immune cells in vivo, mice were immunized with MOG and simultaneously orally administered with synthetic miRNA or bacteria for 7 consecutive days as indicated in results. On day 8 post immunization, cells were collected from the spleen and measured T lymphocytes following established approach (Rezende et al., 2015). Briefly, intracellular cytokine staining was performed by first stimulating cells for 4 h with PMA (phorbol 12-myristate 13-aceate; 50 ng/ml; Sigma-Aldrich) and ionomycin (1 μM; Sigma-Aldrich) and a protein-transport inhibitor containing monensin (1 μg/ml GolgiStop; BD Biosciences) before detection by staining with antibodies. Surface markers were stained for 25 min at 4° C. in Mg2+ and Ca2+ free HBSS with 2% FCS, 0.4% EDTA (0.5 M) and 2.5% HEPES (1 M) then were fixed in Cytoperm/Cytofix (eBioscience), permeabilized with Perm/Wash Buffer (eBiosciences). Flow-cytometric acquisition was performed on a Fortessa (BD Biosciences) by using DIVA software (BD Biosciences) and data were analyzed with FlowJo software versions 10.4.1 (TreeStar). Surface staining antibodies included: Alexa Fluor® 700 anti-CD3 (17A2; 1:100; Biolegend), BV605-anti-CD4 (RM4.5; 1:300; BD Bioscience), PE-anti-Vbeta11 (RR3-15; 1:200; BD Pharmingen). Intracellular staining antibodies used: FITC-anti-FoxP3 (FJK-16s; 1:100; eBioscience), BV421-anti-IFN-γ (XMG1.2; 1:300; Biolegend), PE-Cy7-IL-17A (eBio17B7; 1:100; eBioscience).

To investigate which intestinal cells expressed miR-30d during EAE-induction (FIG. 7), colonic tissue was collected 10 days post MOG/CFA or OVA/CFA immunization. Colonic epithelial cells and lamina propria cells were isolated following the established protocol (Moreira et al., 2019). Colonic homogenates were incubated with DTT as described (Moreira et al., 2019) and were separated into CD45- and CD45+ fractions using CD45 Microbeads (Order number: 130-052-301, Miltenyi Biotec). Epithelial cells from the CD45− fraction were further sorted out by staining with 7-AAD for dead cell exclusion and FITC-anti-CD3 (500A2; 1:100; Biolegend), APC-anti-CD326 (Ep-CAM) (G8.8; 1:100; Biolegend), APC-anti-CD324 (E-Cadherin) (DECMA-1; 1:100; Biolegend) and APC-anti-pan Cytokeratin (C-11; 1:100; Invitrogen). CD45+ fraction was further sorted for αβ+ T cells (7-AAD−, CD3+, TCRγδ-, TCRβ+) and γδ+ T cells (7-AAD−, CD3+, TCRγδ+) by staining with 7-AAD for dead cell exclusion and FITC-anti-CD3 (500A2; 1:100; Biolegend), PE-anti-TCRγδ (GL3; 1:100; Biolegend) and Brilliant Violet 605-anti-TCR-β (H57-597; 1:100; Biolegend). CD45+ fraction was separated from the colonic lamina propria isolates using CD45 Microbeads and was further stained with 7-AAD and APC-conjugated epithelium dump channel (anti-CD326 (Ep-CAM) (G8.8; 1:100; Biolegend), anti-CD324 (E-Cadherin) (DECMA-1; 1:100; Biolegend), anti-pan Cytokeratin (C-11; 1:100; Invitrogen)), PerCP-conjugated dump channel (anti-NK1.1 (PK136; 1:100; Biolegend), anti-Ly-6G (1A8; 1:100; Biolegend), anti-B220 (RA3-6B2; 1:100; Biolegend), anti-CD317 (927; 1:100; Biolegend), anti-CD3 (145-2C11; 1:100; Biolegend)), FITC-anti-CD45 (30-F11; 1:100; Biolegend), PE-anti-CX3CR1 (SA011F11; 1:100; Biolegend), BV605-anti-F4/80 (BM8; 1:100; Biolegend), BV605-anti-CD64 (X54-5/7.1; 1:100; Biolegend), PE/Cy7-anti-CD11c (N418; 1:100; Biolegend) and AF700-anti-I-A/I-E (MHCII) (M5/114.15.2; 1:100; Biolegend) to sort for macrophages (7-AAD− APC− PerCP− CD45+ F4/80+ CD64+ CX3CR1+) and dendritic cells (7-AAD− APC− PerCP− CD45+ F4/80− CD64− MHCII hi+CD11c+).

To investigate the effect of A. muciniphila on Foxp3+ Treg induction (FIG. 6E-G), CD11c+ dendritic cells (DCs) isolated from the mesenteric lymph nodes (MLNs) of naive mice were first enriched with UltraPure CD11c MicroBeads (order No. 130-108-338, Miltenyi Biotec), and then sorted by gating 7-AAD− PerCP− CD45+F4/80− CD64− CD11c+ cells. Antibodies used for sorting were: PerCP-conjugated dump channel (anti-TER-119 (TER-119), anti-NK1.1(PK136), anti-CD19(6D5), anti-Ly-6G (1A8), anti-CD3e(145-2C11), all at 1:300 dilution; Biolegend), APC-anti-CD45 (30-F11; 1:300; Biolegend), FITC-anti-F4/F80 (BM8; 1:100; Biolegend), FITC-anti-CD64 (X54-5/7.1; 1:100; Biolegend) and PE-anti-CD11c (N418; 1:200; Biolegend). Naive CD4+ T cells were isolated from the spleen of Foxp3^{GFP+} mice using Naïve CD4+ T cell Isolation Kit (Order No. 130-104-453, Miltenyi Biotec).

In Vitro Induction of Foxp3 Tregs with A. muciniphila

To investigate the effect of A. muciniphila on Foxp3+ Treg induction (FIG. 6E-G), Foxp3+ Tregs were induced from naïve CD4+ T cells that were purified from the splenocytes of Foxp3^{GFP+} mice as described above in the presence of TGF-01 (2 ng/ml, R&D Systems) and IL-2 (10 ng/ml, R&D Systems) for 3 days. In some experiments (FIG. 6E), 1 μl/well (200 μl) of the inactivated OD600=1 A. muciniphila or E. coli were added to directly stimulate naïve CD4 T cells. In some experiments (FIG. 6F-G), 1 μl/well of the inactivated OD600=1 A. muciniphila or E. coli were added to DCs, isolated from MLN of naïve mice, described above, for 24 hours. Stimulated DCs were harvested for RNA isolation (FIG. 6G), or co-cultured with naïve CD4 T cells to expand Tregs for additional 3 days by adding naïve CD4+T from Foxp3^{GFP+} mice to the stimulated DCs, at a DC: naïve CD4+ T cell ratio of 1:10 (FIG. 6F).

Cellular RNA Isolation and qPCR Quantification of Transcripts of miRNA and Cytokine Genes

To determine miR-30d changes in different cells (epithelial cells, macrophages, dendritic cells, TCRαβ+IELs, TCRγδ+IELs) in the gut of MOG/CFA- or OVA/CFA-immunized mice (FIG. 7), and to determine the expression of Tgfb, Il6 and Il1b mRNAs in A. muciniphila-treated DCs (FIG. 6G), total RNA (including miRNA) was extracted from sorted cells or cultured DCs using mirVana™ miRNA isolation kit (catalog number: AM1560, Ambion®) following manufacturer's protocol. qPCR was performed to detect miR-30d using TaqMan® MiRNA Reverse Transcription (Applied Biosystems) and Taqman Universal PCR Master Mix according to the manufacturer's protocol. The input of total RNA per sample was 5 ng. The TaqMan® MiRNA Assay IDs (Applied Biosystems) were: snoRNA135 (inner control, assay ID: 001230), the hsa-miR-30d-5p (assay ID: 000420). The TaqMan™ Gene Expression Assay IDs (Applied Biosystems) were: Gapdh (Assay ID: Mm99999915_g1, reference gene), Tgfb1 (Assay ID: Mm01178820_m1), 116 (Assay ID: Mm00446190_m1), Il1beta (Assay ID: Mm00434228_m1).

In Situ Hybridization Detection of miR-30d Entered in A. muciniphila A. muciniphila was cultured in 1 ml medium of BHI w/o dextrose plus 0.2% mucin in the presence of 5 μM synthetic miR-30d mimics or scramble for 18 hours to an exponential phase. Bacterial cells were spin down at 12000 rpm. Washed twice with ice cold PBS and fixed with 4% PFA/0.25% Glutaraldehyde. 100 nm cryosection were proceeded on nickel grids and carried out for in situ hybridization using a 5′-DIG and 3′-DIG dual labeled probe for miR-30d (Cat #YD00613716-BEG, Product #339112, Qiagen) and 10 nm immuno gold-conjugated anti-Digoxigenin antibody (Cat #25399, Electron Microscopy Sciences) following the manufacturer's protocol. Sections on grids were imaged using Tecnai G2 Spirit BioTWIN Transmission Electron Microscope.

Statistical Analysis

Unless otherwise indicated, data were analyzed using GraphPad Prism 7.0c software (San Diego, Calif., USA). The differences between two groups were analyzed with Student's t-test with proper correction. The differences between more than two groups were analyzed using ANOVA with multiple comparisons test. A two-sided p-value of ≤0.05 was considered as significant. Unless otherwise specified, results were expressed as mean±SEM.

### Example 1. Gut Microbiome Changes During EAE

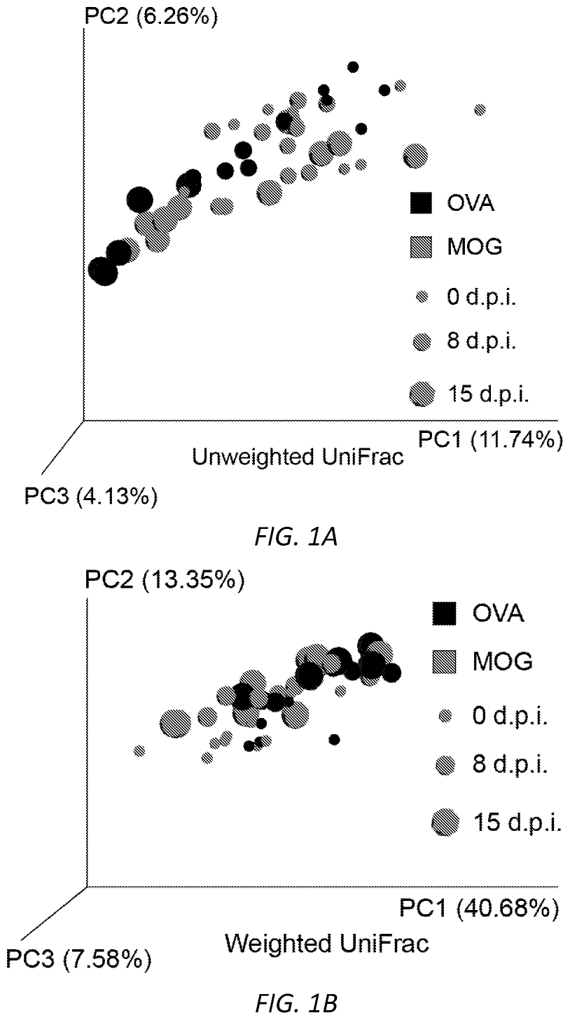

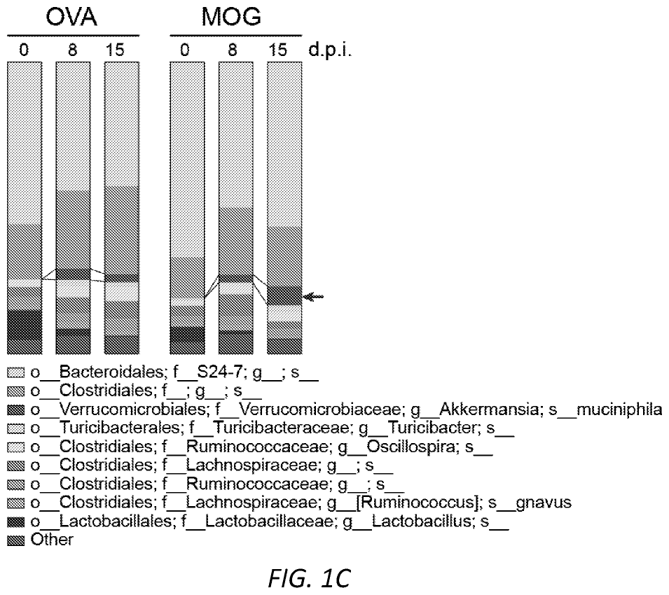

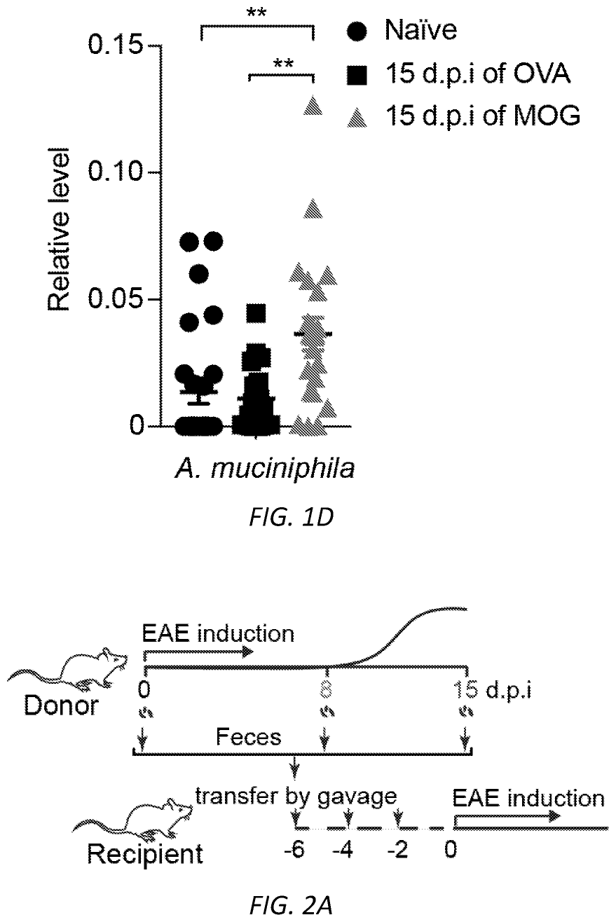

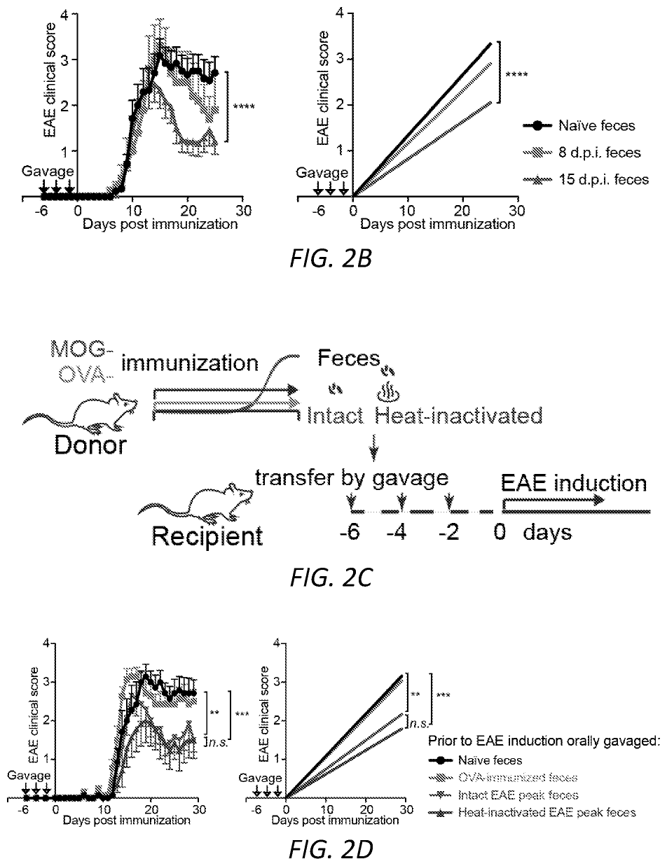

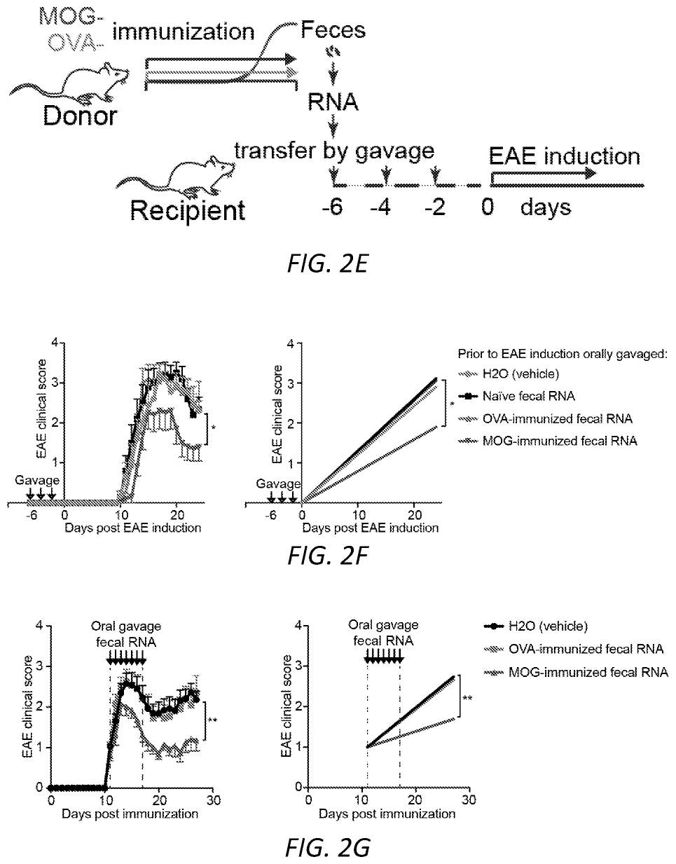

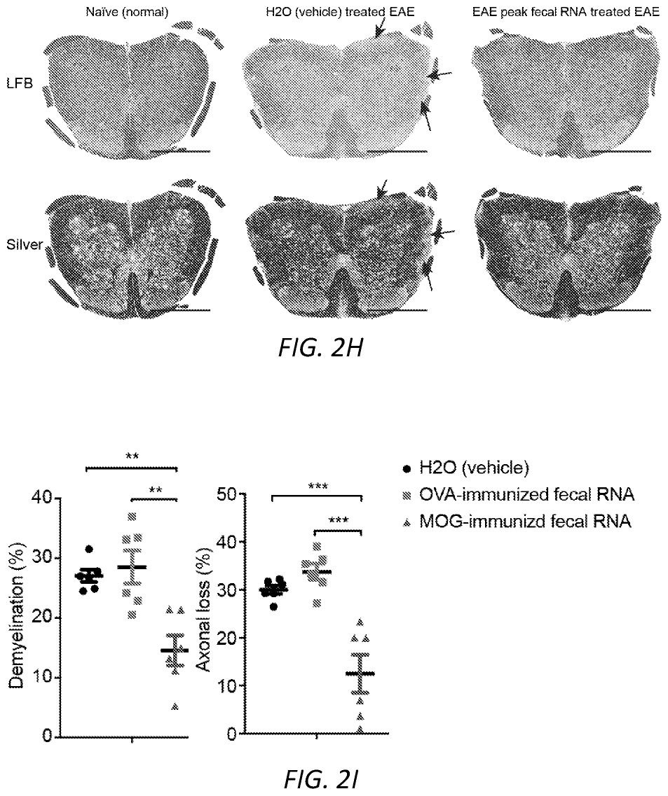

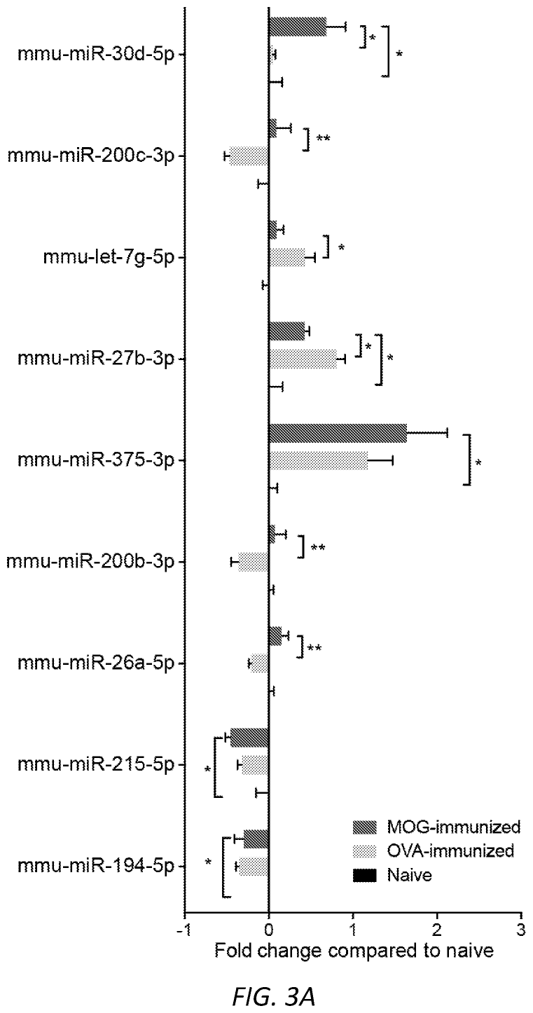

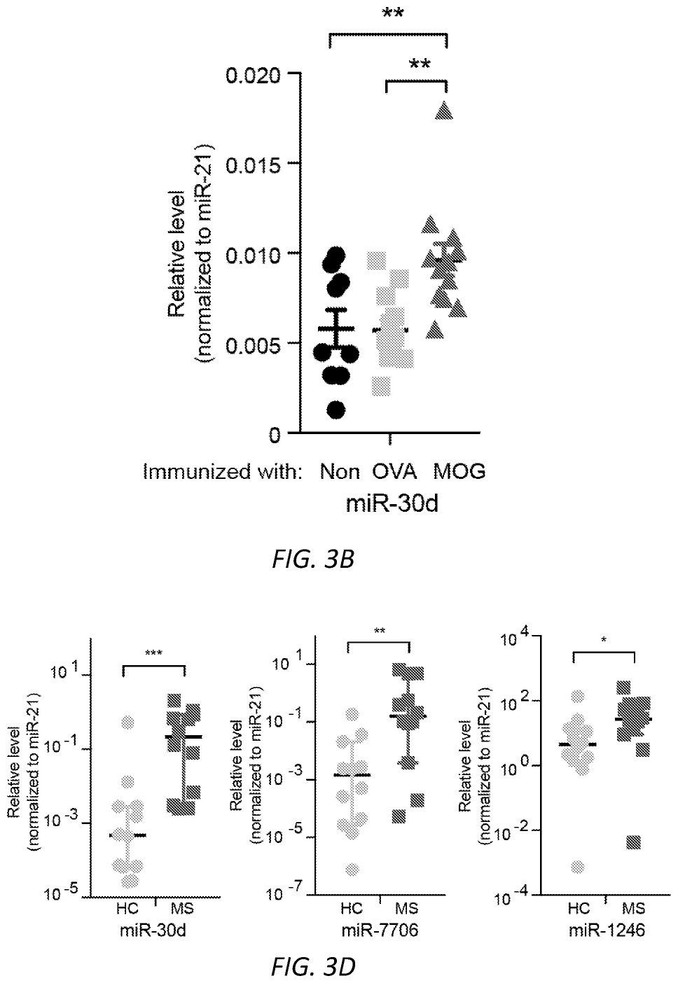

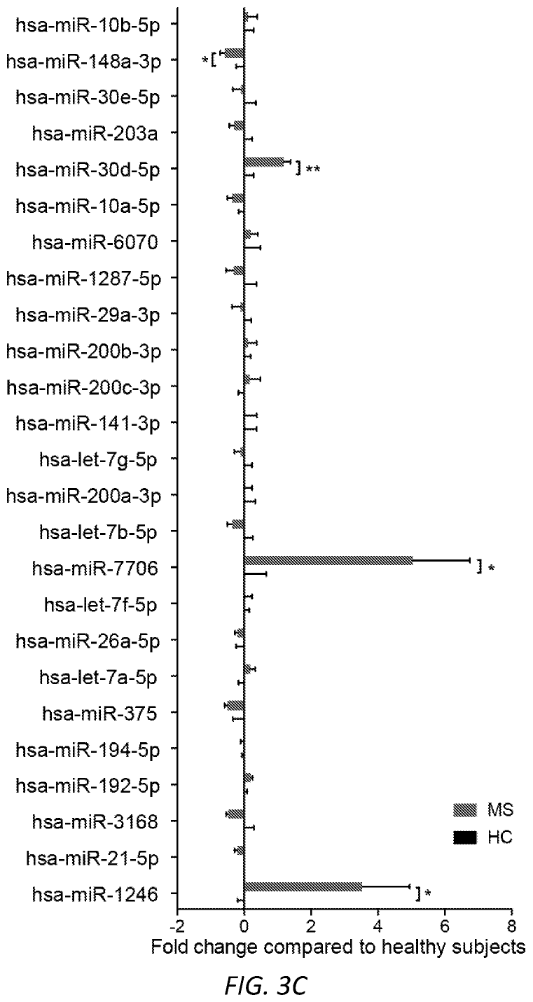

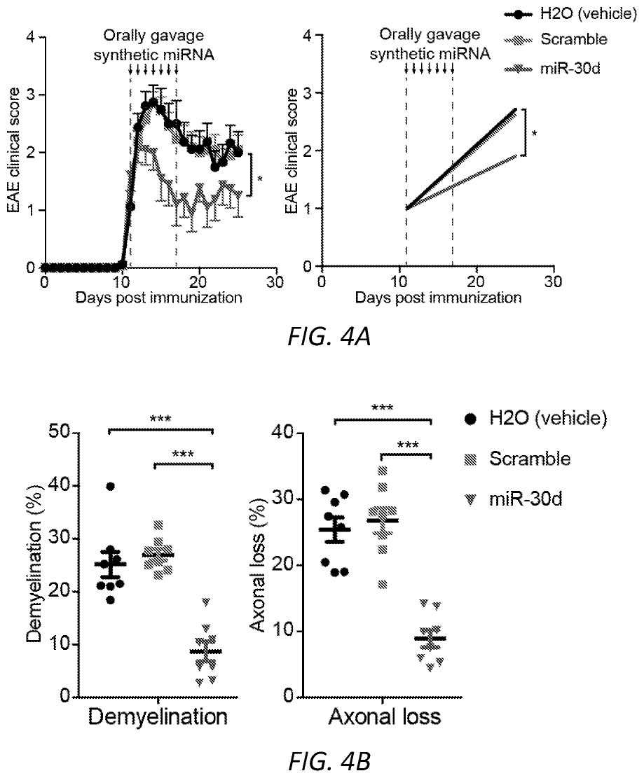

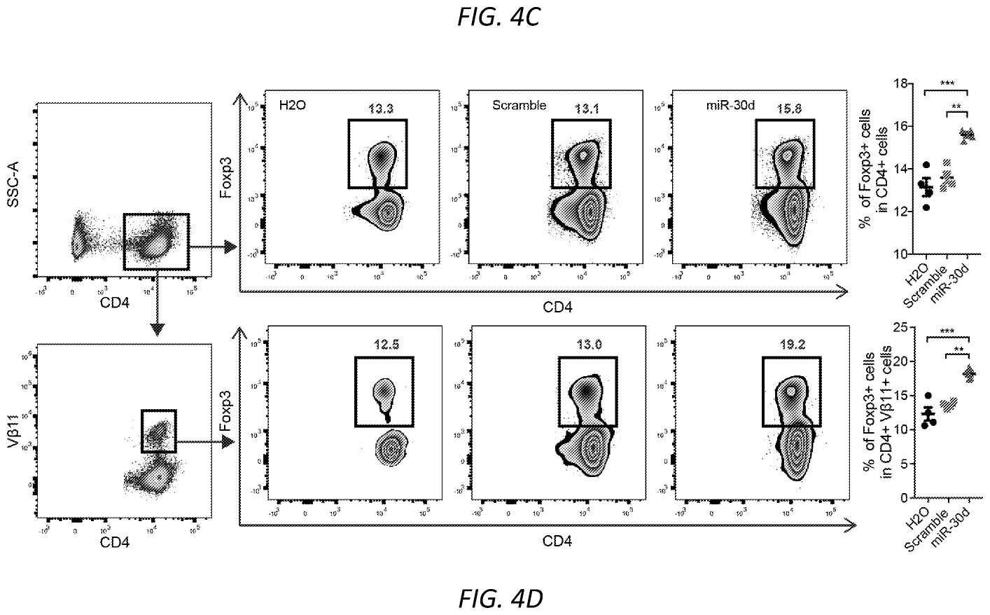

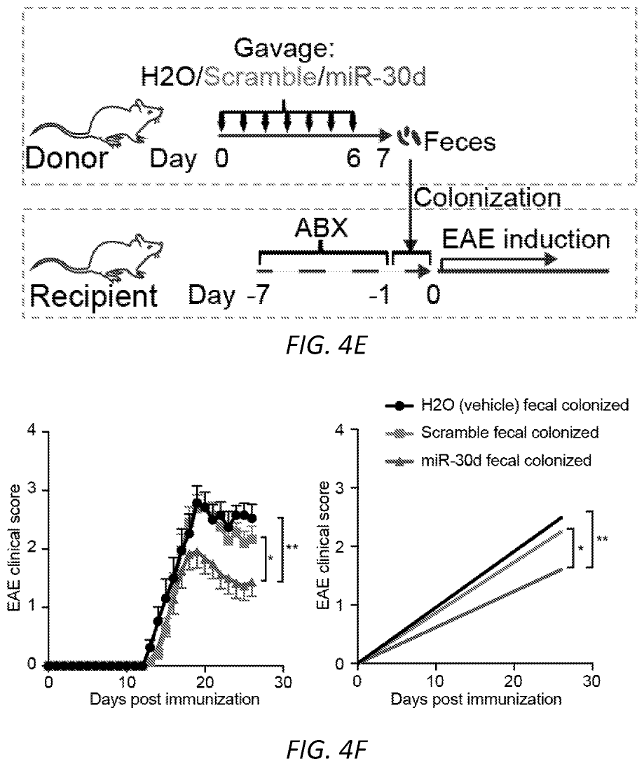

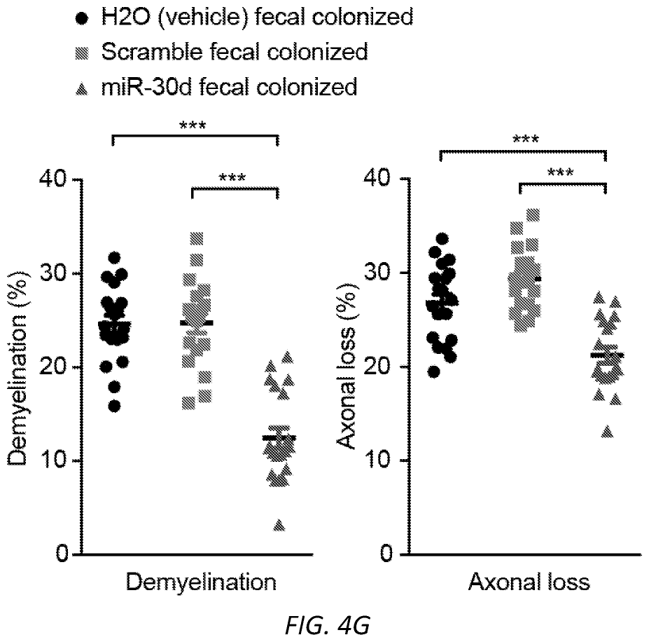

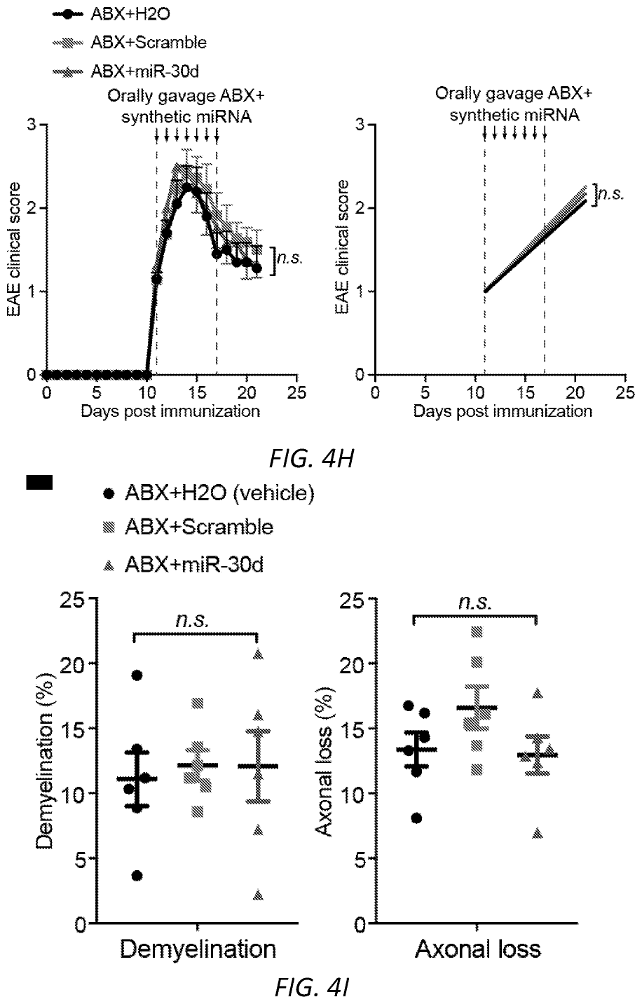

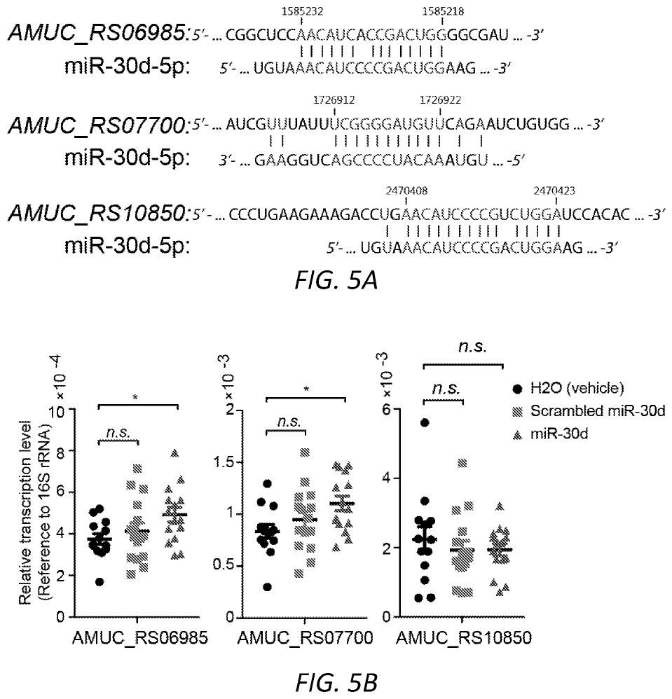

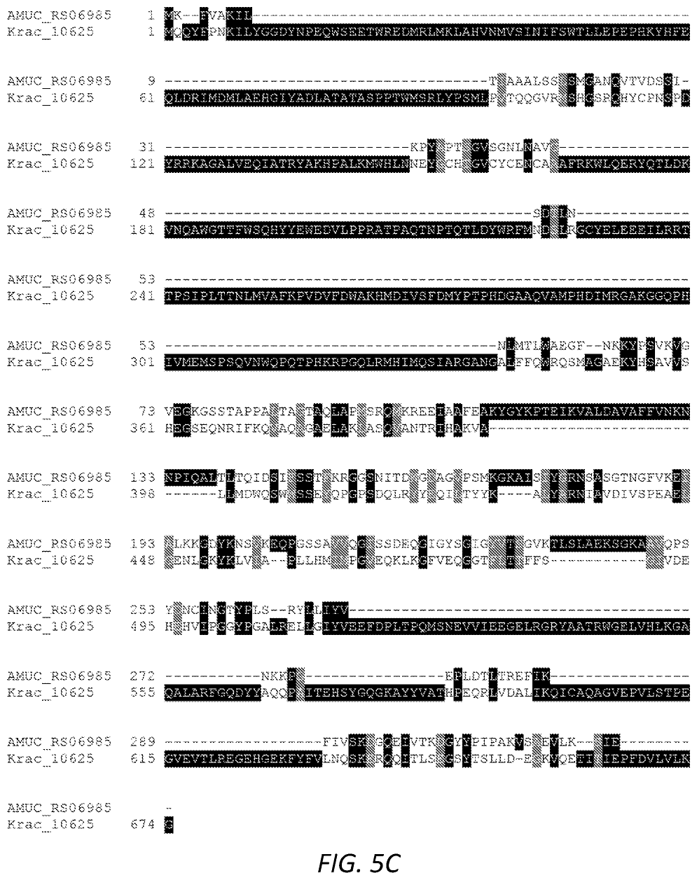

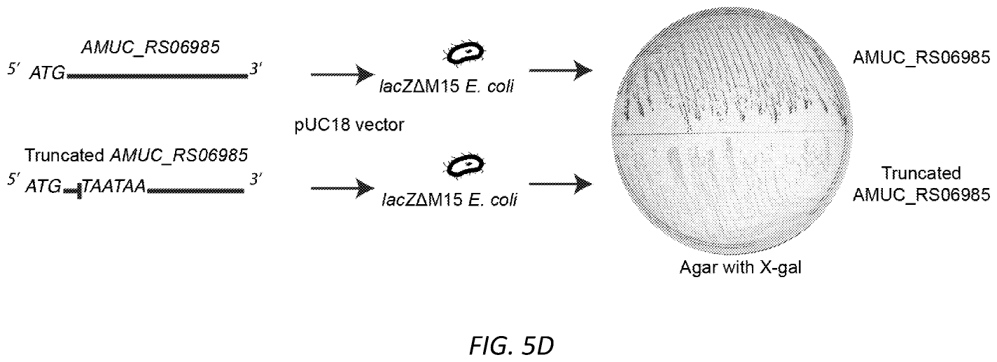

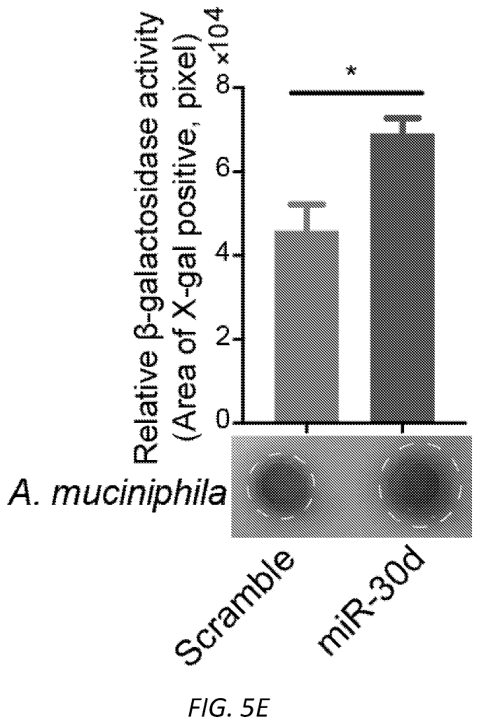

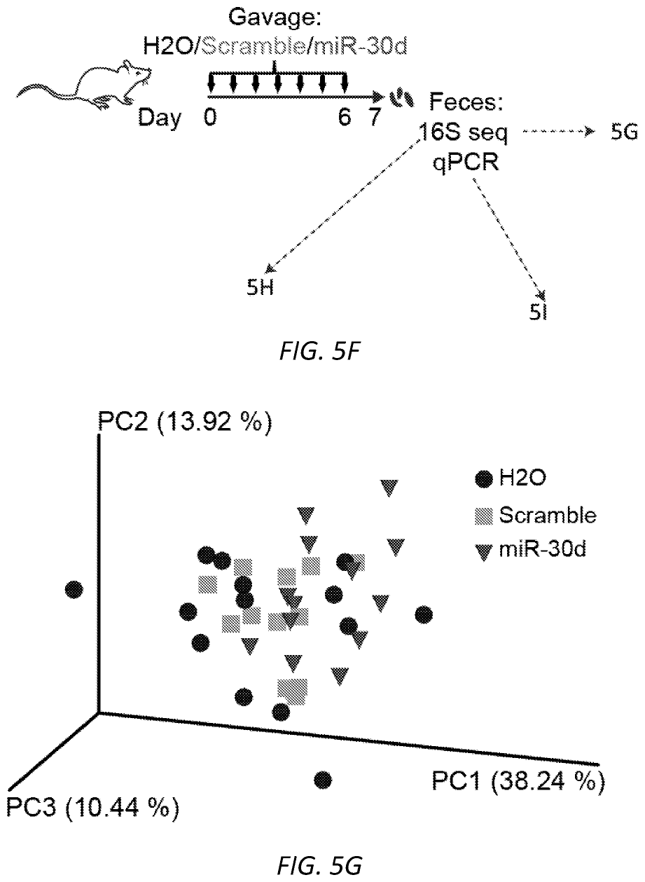

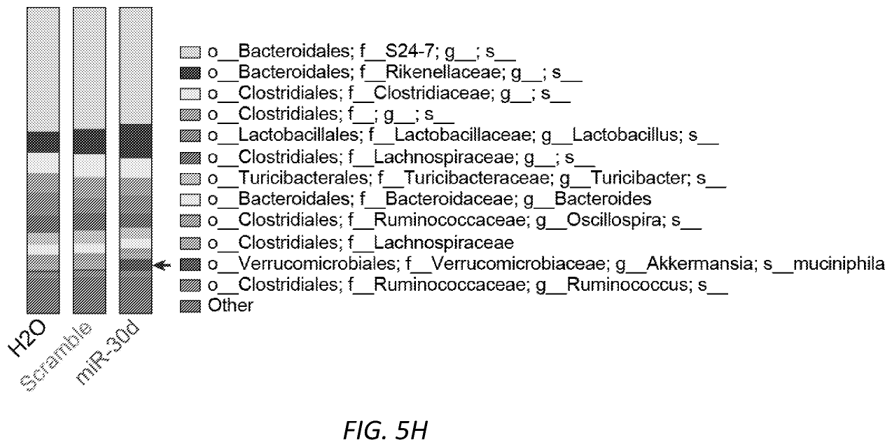

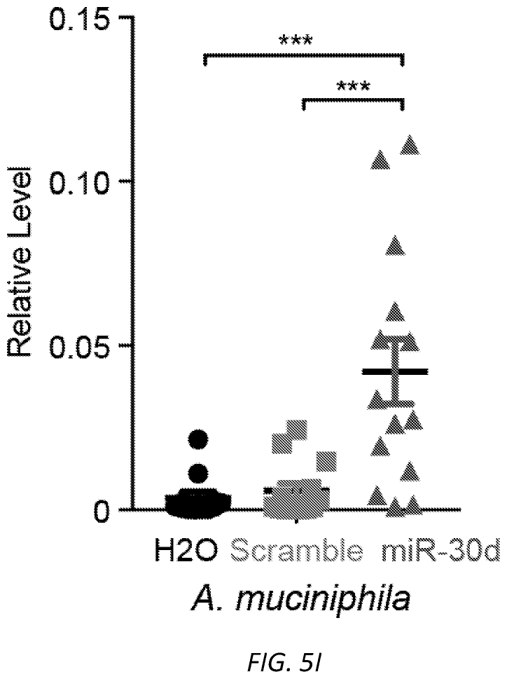

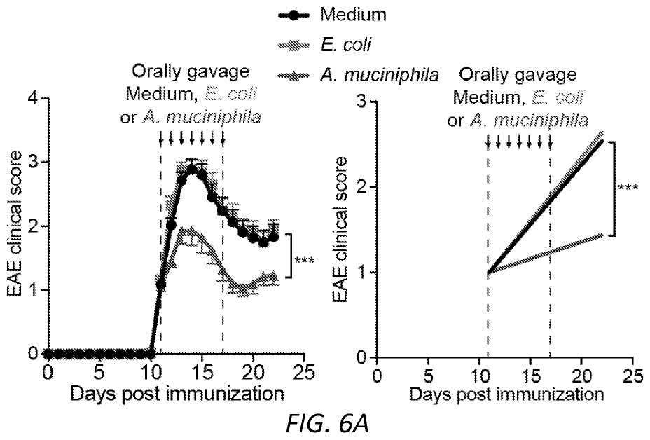

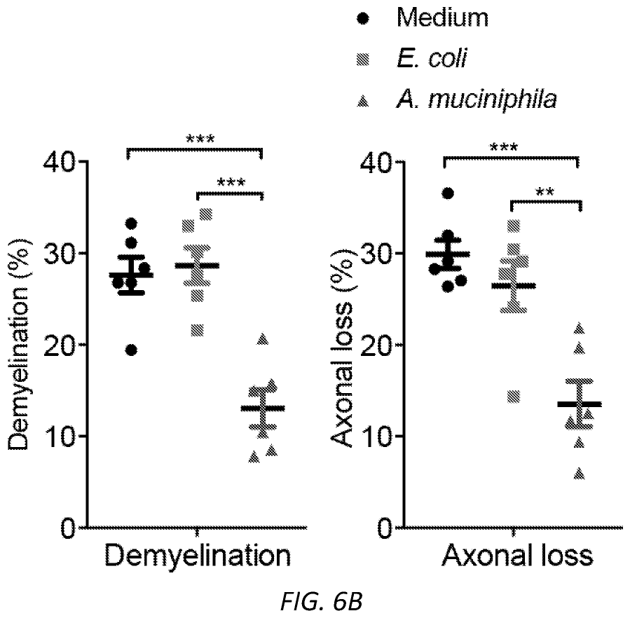

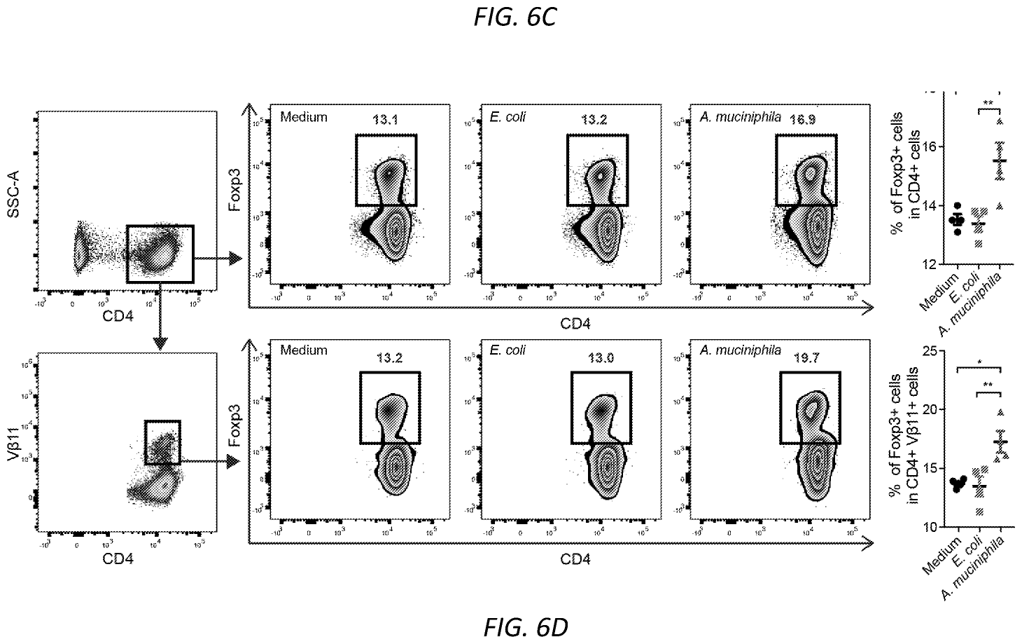

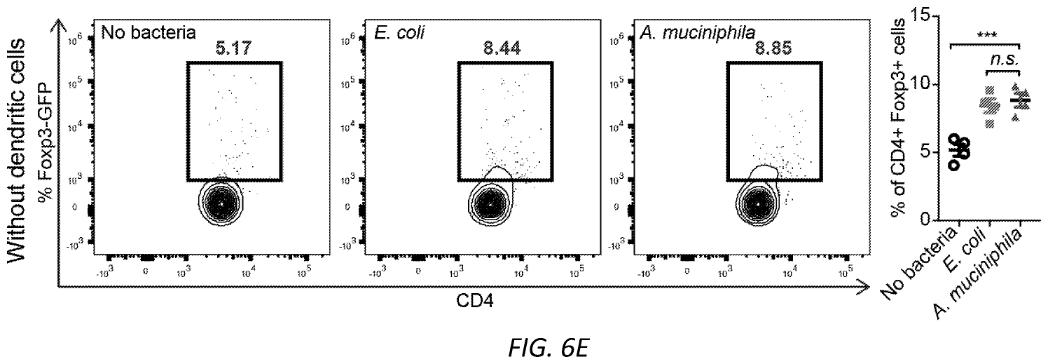

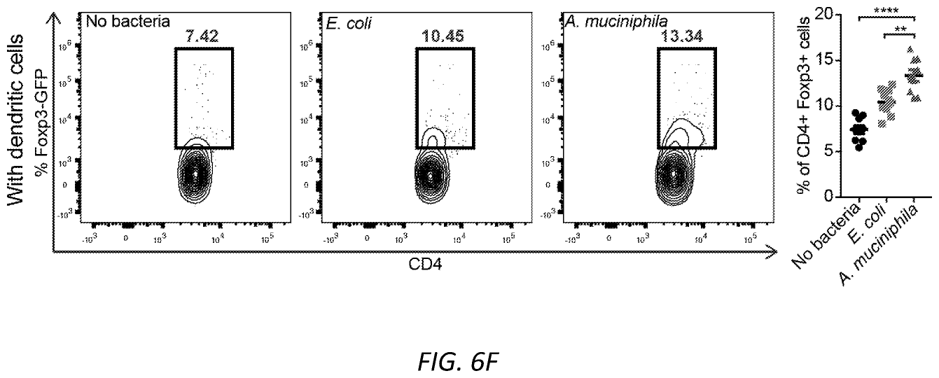

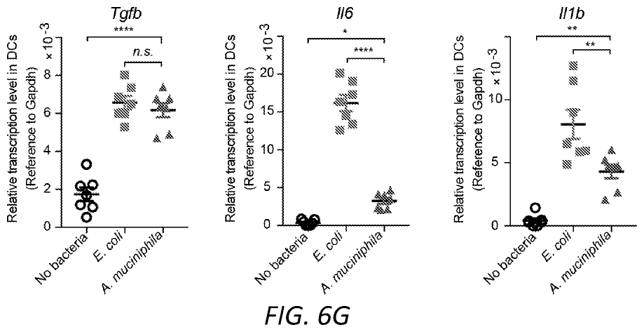

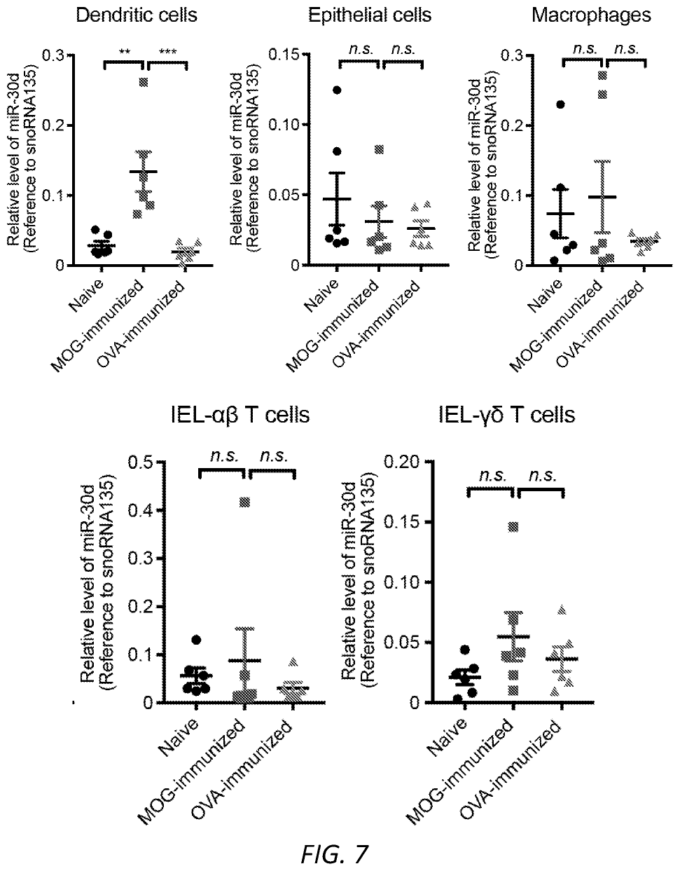

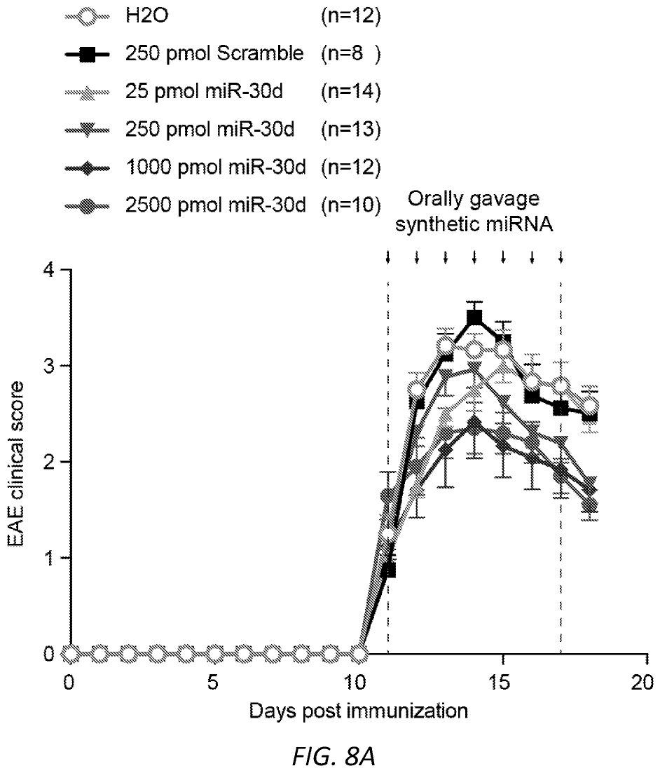

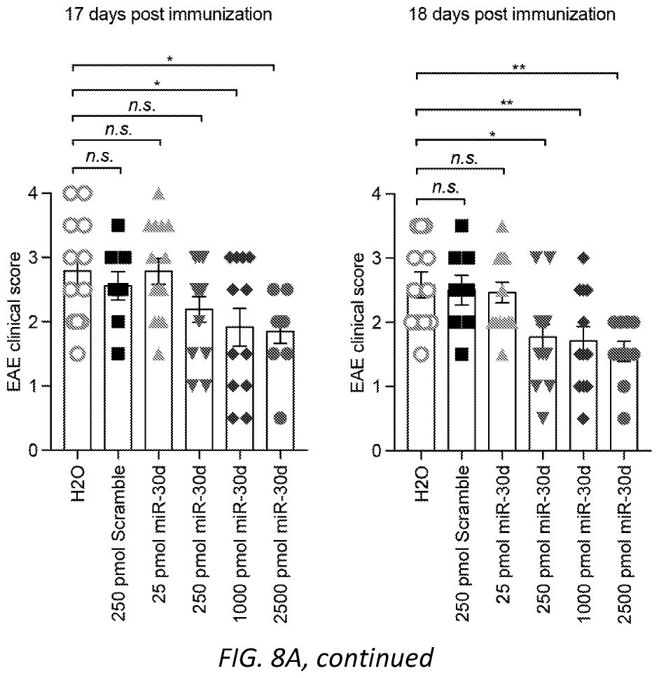

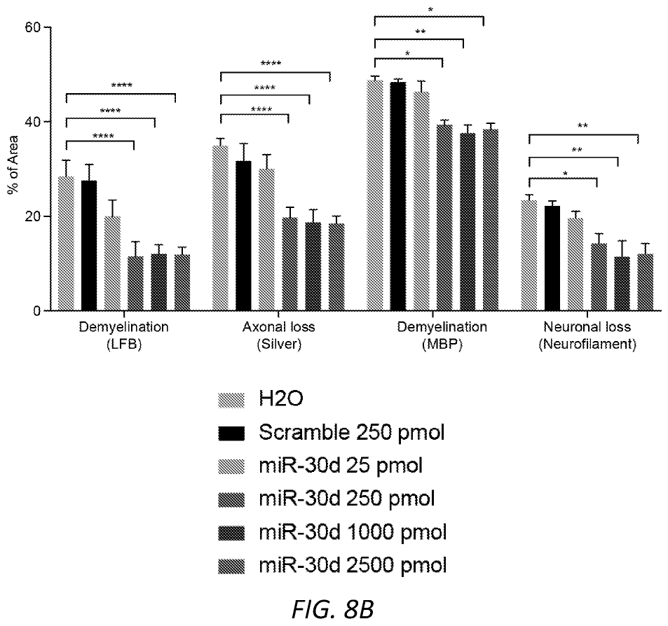

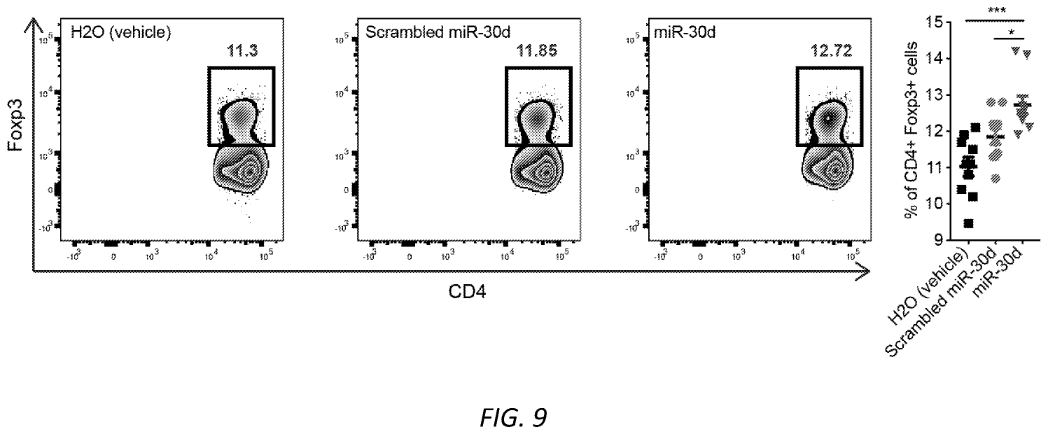

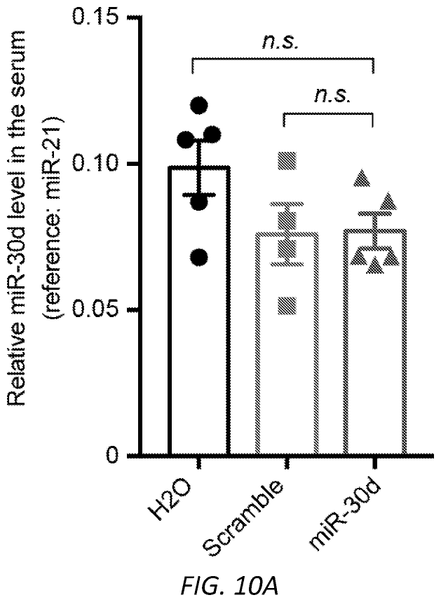

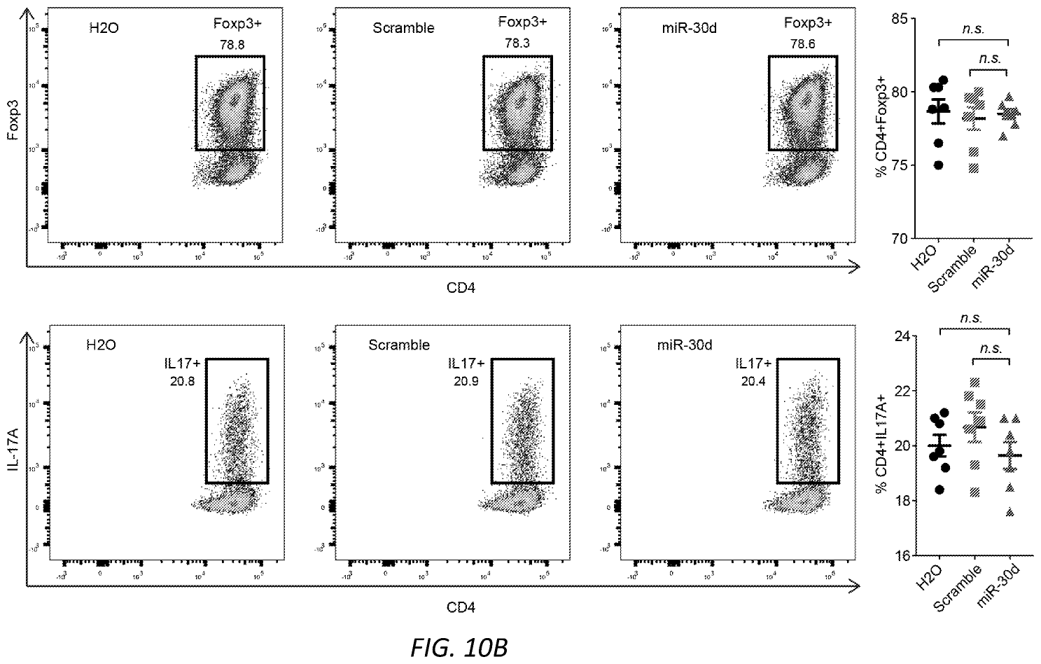

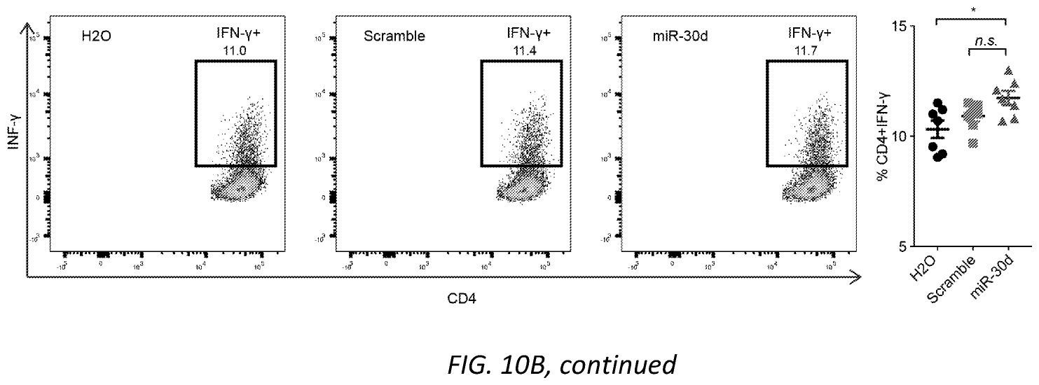

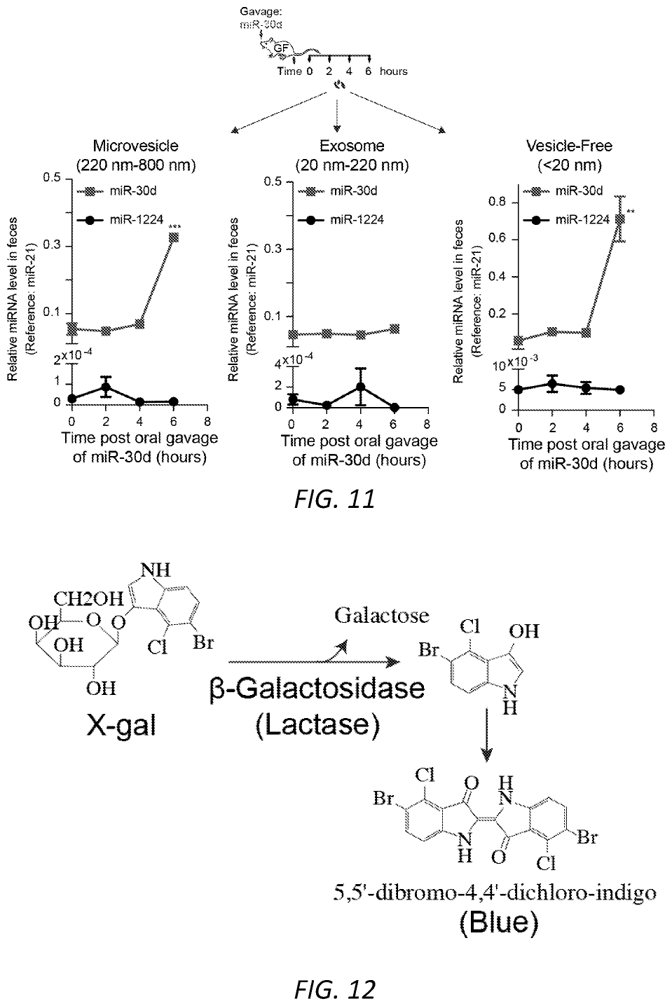

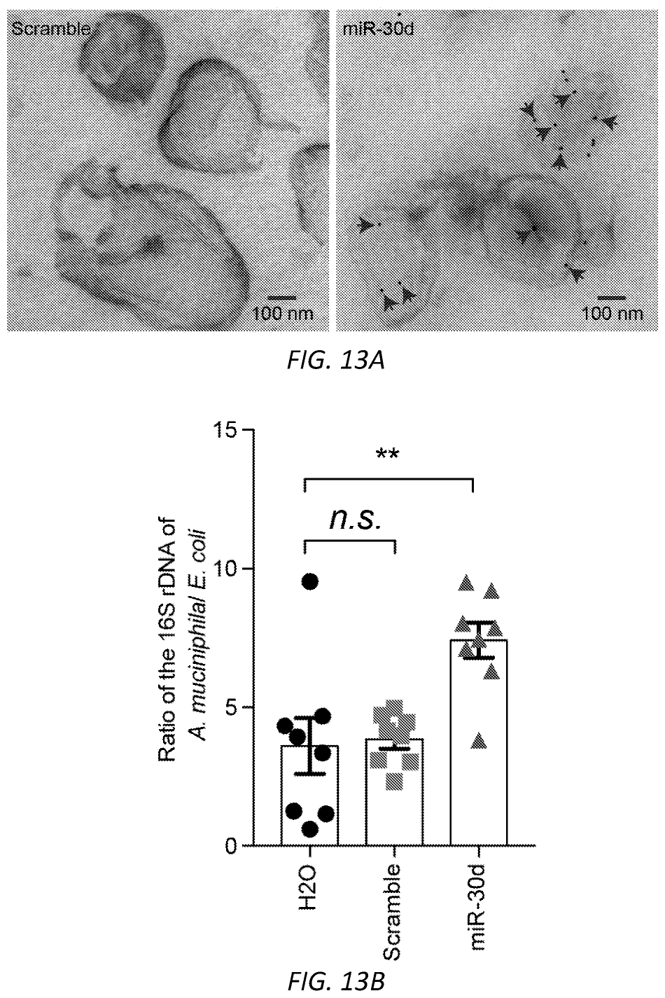

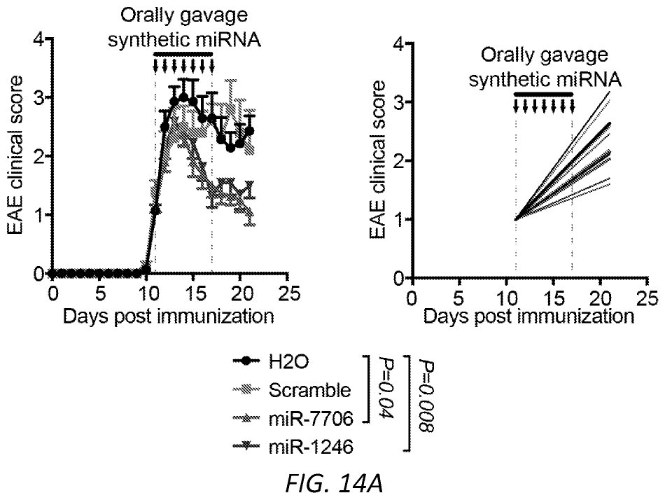

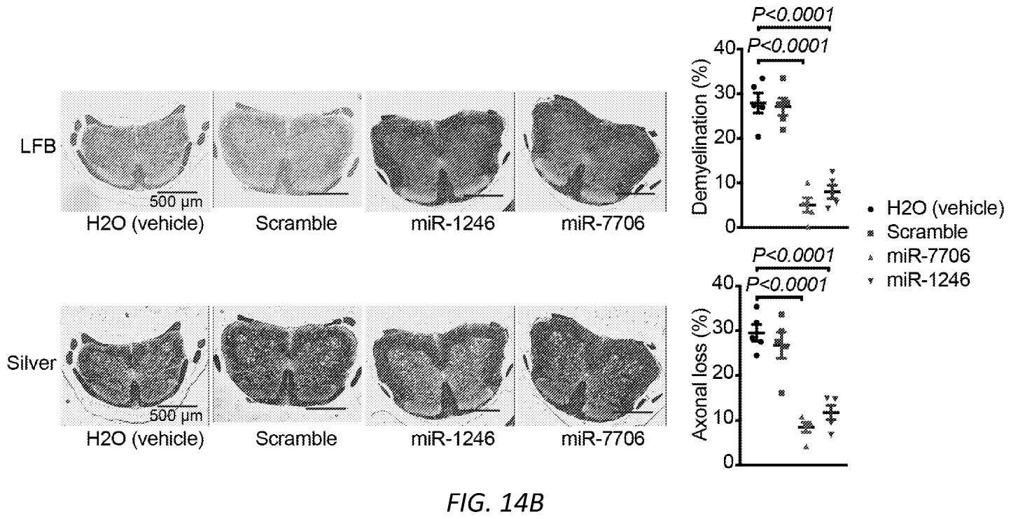

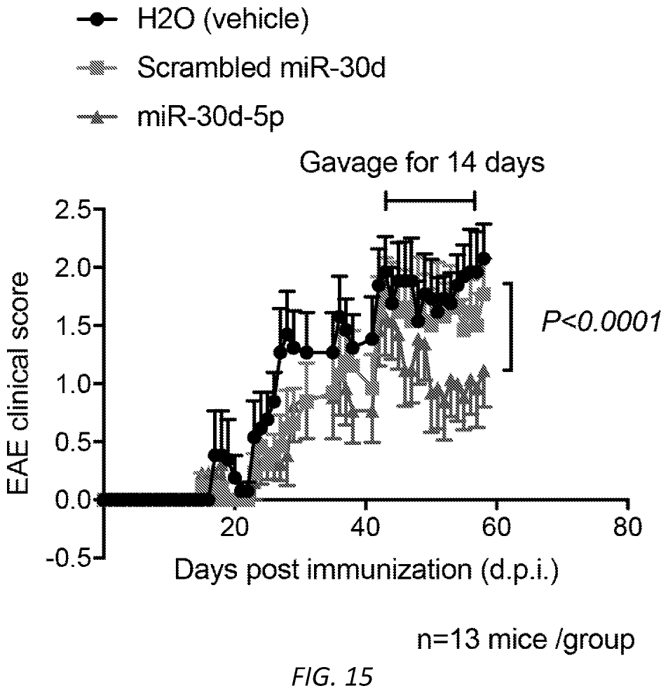

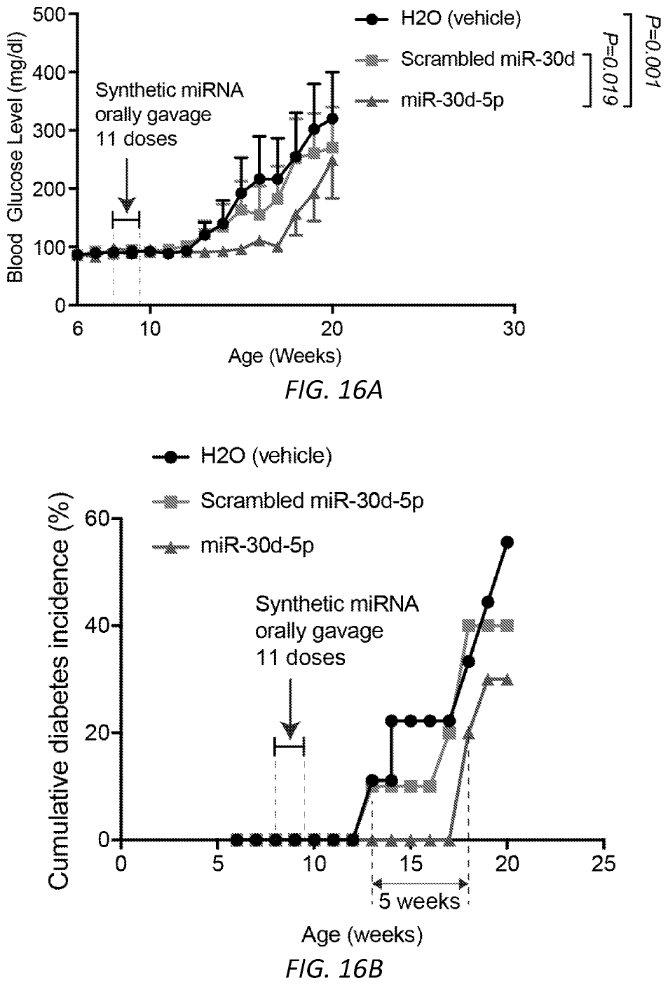

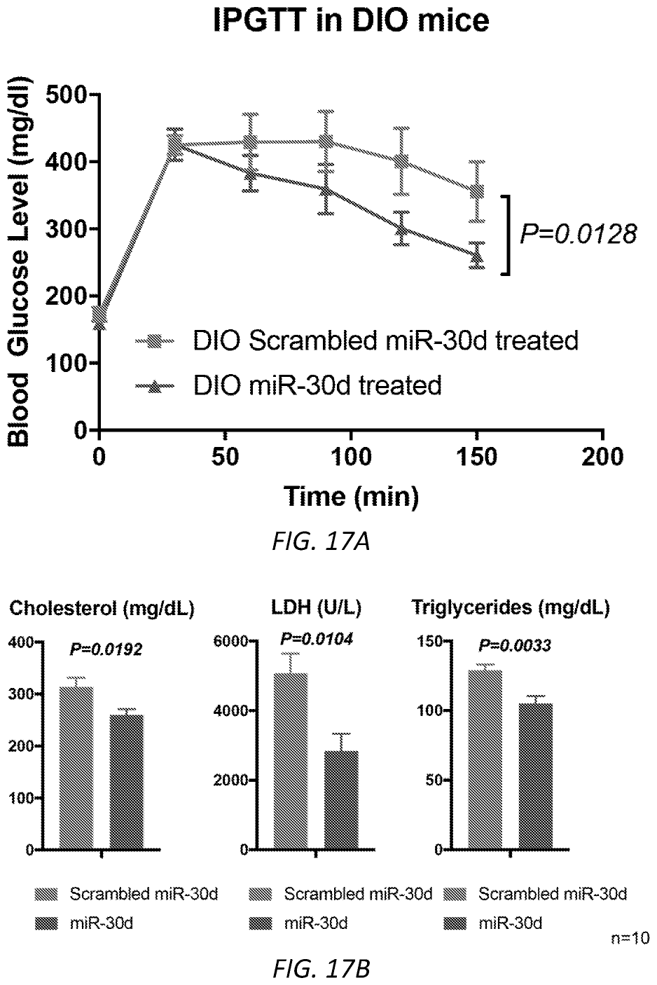

Commensal microbiome is essential for the development and function of the host immune system (Belkaid and Hand, 2014). EAE is a primary animal model of MS (Robinson et al., 2014). Mouse model of spontaneous relapsing-remitting MS does not develop EAE when raised under germ-free condition (Berer et al., 2011) and mice orally treated with antibiotics have less severe EAE (Ochoa-Reparaz et al., 2009). We and others have detected an altered gut microbiome in MS (Berer et al., 2017; Cekanaviciute et al., 2017; Chen et al., 2016; Jangi et al., 2016; Tremlett et al., 2016). To investigate the microbiome composition in EAE, we induced EAE in C57BL/6 mice by immunization with myelin oligodendrocyte glycoprotein (MOG) emulsified with Freund's complete adjuvant (CFA). Control mice were immunized with ovalbumin (OVA)/CFA emulsion. Fecal specimens were collected at the time of immunization, 8 days post immunization (8 d.p.i., prior to onset of EAE), and 15 d.p.i. (peak EAE disease); we performed 16S rDNA sequencing to analyze the microbiome. An unweighted (FIG. 1A) and a weighted (FIG. 1B) UniFrac beta-diversity metric assessment of overall microbial structure did not reveal significant difference between MOG-induced EAE and OVA-immunized control. We next investigated whether the relative abundances of the microbiome differed between MOG- and OVA-immunized mice at a species-level taxonomy. We found that A. muciniphila was increased in the feces from MOG-induced EAE mice, but not from OVA/CFA immunized mice, on day 15 (FIG. 1C and Table 1), which we confirmed by quantitative PCR (qPCR) (FIG. 1D). Of note, A. muciniphila was also found to be increased in the stool of untreated MS patients compared to healthy subjects from multiple studies including ours (Berer et al., 2017; Cekanaviciute et al., 2017; Jangi et al., 2016; Tremlett et al., 2016).

TABLE 1
Gut microbiome during EAE

| | Average | Average | Average | Average | Average | Average |
|---|---|---|---|---|---|---|
| | D 0 | D 8 | D 15 | D 0 | D 8 | D 15 |
| Taxonomy | OVA | OVA | OVA | MOG | MOG | MOG |
| A. | 55.61% | 44.16% | 42.66% | 66.93% | 49.97% | 56.59% |
| B. | 18.90% | 26.72% | 30.07% | 14.12% | 22.97% | 20.33% |
| C. | 0.02% | 3.86% | 2.78% | 0.02% | 2.69% | 6.53% |
| D. | 0.30% | 2.11% | 1.83% | 0.86% | 0.89% | 2.76% |
| E. | 2.24% | 3.82% | 4.67% | 1.66% | 3.08% | 2.72% |
| F. | 3.51% | 5.64% | 5.99% | 3.79% | 7.60% | 2.64% |
| G. | 2.83% | 3.05% | 3.90% | 2.46% | 2.73% | 2.59% |
| H. | 1.62% | 2.07% | 1.84% | 0.95% | 2.13% | 0.80% |
| I. | 10.30% | 2.54% | 0.44% | 5.33% | 1.26% | 0.39% |
| J. | 4.66% | 6.04% | 5.82% | 3.89% | 6.67% | 4.65% |

Taxonomy for Table 1:
A. k_Bacteria; p_Bacteroidetes; c_Bacteroidia; o_Bacteroidales; f_S24-7; g_; s_
B. k_Bacteria; p_Firmicutes; c_Clostridia; o_Clostridiales; f_; g_; s_
C. k_Bacteria; p_Verrucomicrobia; c_Verrucomicrobiae; o_Verrucomicrobiales; f_Verrucomicrobiaceae; g_Akkermansia; s_muciniphila
D. k_Bacteria; p_Firmicutes; c_Bacilli; o_Turicibacterales; f_Turicibacteraceae; g_Turicibacter; s_
E. k_Bacteria; p_Firmicutes; c_Clostridia; o_Clostridiales; f_Ruminococcaceae; g_Oscillospira; s_
F. k_Bacteria; p_Firmicutes; c_Clostridia; o_Clostridiales; f_Lachnospiraceae; g_; s_
G. k_Bacteria; p_Firmicutes; c_Clostridia; o_Clostridiales; f_Ruminococcaceae; g_; s_
H. k_Bacteria; p_Firmicutes; c_Clostridia; o_Clostridiales; f_Lachnospiraceae; g_[Ruminococcus]; s_gnavus
I. k_Bacteria; p_Firmicutes; c_Bacilli; o_Lactobacillales; f_Lactobacillaceae; g_Lactobacillus; s_
J. Other

### Example 2. Orally Transfer of Feces and Fecal miRNA from Peak EAE Ameliorated EAE

To investigate whether the gut microbiome from mice with EAE had pathogenic properties, we transplanted feces from EAE mice into naïve animals that were then immunized with MOG for EAE induction (FIG. 2A). We found that transplantation of feces obtained during peak EAE (day 15) ameliorated disease in recipient mice as compared to transplantation of feces from healthy mice (day 0) (FIG. 2B). A similar trend was observed using feces obtained from EAE animals on day 8 (FIG. 2B). To determine whether live bacteria in peak EAE feces were responsible for the effects we observed, we heat-inactivated feces from peak EAE donor mice prior to transfer (FIG. 2C). We found that both intact and heat-inactivated peak EAE feces ameliorated EAE in a similar fashion (FIG. 2D), indicating that live microbes were not required and a heat resistant component in the peak EAE feces improved EAE. No effect was observed when feces from OVA-immunized mice were transferred (FIG. 2D). We and others have reported that host miRNAs can modulate bacterial transcripts and growth (Liu et al., 2016; Teng et al., 2018). MicroRNAs are heat resistant (Jung et al., 2010); thus to determine whether fecal miRNAs were responsible for the effects we observed, we purified fecal RNA from peak EAE feces and administered it orally to recipient mice prior to immunization for EAE (FIG. 2E). We found that the purified peak EAE fecal RNA ameliorated EAE similarly to peak EAE feces (FIG. 2F). No effect was observed with fecal RNA from non-immunized animals or OVA-immunized animals (FIG. 2F). Furthermore, we also observed a disease ameliorating effect, as indicated by decreased clinical EAE score, less demyelination and axonal loss, when fecal RNA was administered at the onset of EAE (FIG. 2G-I) rather than being given prophylactically.

### Example 3. MiR-30d is Enriched in Feces from EAE Animals at Peak Disease and in Feces from Untreated MS Patients

We have previously shown that the majority of RNA components found in the feces are small RNAs, and predominantly miRNAs (Liu et al., 2016). To identify which fecal miRNAs were generated during EAE, we performed small RNA sequencing in which we measured fecal RNA from peak EAE, OVA-immunized and non-immunized mice. We found that peak EAE mice had an increased level of miR-30d-5p (miR-30d) compared to OVA-immunized and non-immunized mice (FIG. 3A and Table 2). We confirmed the increased level of miR-30d in peak EAE feces by qPCR (FIG. 3B). We then asked whether specific fecal miRNAs were increased in subjects with MS. We investigated untreated relapsing-remitting MS subjects and compared them to age- and sex-matched controls. Using small RNA sequencing followed by qPCR confirmation we found that miR-30d, miR-7706 and miR-1246 were higher in untreated MS vs. healthy control (FIG. 3C-D and Table 3). Thus, fecal miR-30d was increased in both peak EAE and subjects with MS.

To determine which cells were responsible for the upregulation of miR-30d and whether this upregulation was specific for MOG immunization, we immunized mice with either MOG or OVA emulsified in CFA and measured miR-30d in DCs, epithelial cells, macrophages, αβ T cells and γδ T cells in the colon. We found that only DCs upregulated miR-30d and that this effect was dependent on MOG immunization, as non-immunized mice or OVA-immunized mice did not show increased miR-30d expression (FIG. 7). Thus, colonic DCs are responsible for miR-30d upregulation upon MOG immunization.

TABLE 2
Fecal miRNA in OVA- vs MOG-immunized mice, p < 0.05

| | | | Average | T test MOG- | T test MOG- |
|---|---|---|---|---|---|
| | Average Non- | Average OVA- | MOG- | vs Non- | vs OVA- |
| miRNA/counts\Sample ID | immunized | immunized | immunized | immunized | immunized |
| mmu-miR-141-3p | 19.4812376 | 29.71032778 | 14.401735 | 0.185068967 | 0.002124419 |
| mmu-miR-148b-3p | 7.02518525 | 13.94037862 | 6.9339428 | 0.974807308 | 0.002128156 |
| mmu-miR-26a-5p | 358.8491988 | 285.1381989 | 412.58016 | 0.17873564 | 0.004078527 |
| mmu-miR-152-3p | 2.609313875 | 13.14010277 | 5.7207565 | 0.057980647 | 0.004641836 |
| mmu-miR-7a-5p | 6.95426066 | 41.06382846 | 16.330787 | 0.13377184 | 0.00692056 |
| mmu-miR-3068-3p | 4.952755061 | 12.03630402 | 6.5320682 | 0.535937517 | 0.008654869 |
| mmu-miR-378c | 1.843806221 | 6.838048018 | 2.3805947 | 0.722445405 | 0.009053307 |
| mmu-miR-151-5p | 17.56357672 | 10.99813845 | 15.665468 | 0.690966353 | 0.010286799 |
| mmu-miR-5121 | 0.96980238 | 10.66437932 | 3.1179271 | 0.141518132 | 0.012254126 |
| mmu-miR-27b-3p | 89.76144549 | 161.7832372 | 128.08881 | 0.04058011 | 0.01428429 |
| mmu-miR-378a-3p | 50.42659032 | 120.7021729 | 69.527215 | 0.126663607 | 0.01685987 |
| mmu-miR-200c-3p | 99.3170009 | 53.28374034 | 108.05168 | 0.692941611 | 0.016947971 |
| mmu-let-7i-5p | 28.85037589 | 75.49221623 | 32.051979 | 0.63533454 | 0.019131781 |
| mmu-let-7d-3p | 7.270810767 | 3.840992844 | 9.7325869 | 0.509182992 | 0.019149102 |
| mmu-miR-98-5p | 6.231165563 | 12.60955845 | 6.0566612 | 0.937498511 | 0.023865466 |
| mmu-miR-146b-5p | 1.398479905 | 6.821660106 | 2.6660202 | 0.251194977 | 0.024917691 |
| mmu-miR-200b-3p | 340.7634398 | 217.7737753 | 365.05042 | 0.630077156 | 0.025312065 |
| mmu-miR-30d-5p | 62.79324666 | 65.53185629 | 105.62115 | 0.041485087 | 0.02618502 |
| mmu-miR-29a-3p | 35.08255535 | 59.34824518 | 46.479479 | 0.132602909 | 0.036657319 |
| mmu-miR-340-5p | 2.453942132 | 2.992787846 | 5.3540781 | 0.062144012 | 0.036801622 |
| mmu-let-7g-5p | 117.0013059 | 167.7102222 | 127.38294 | 0.451536931 | 0.042602954 |
| mmu-miR-23a-3p | 70.6081192 | 99.9723709 | 58.087784 | 0.218314961 | 0.048213838 |

TABLE 3
Fecal miRNAs in MS patients vs healthy subjects, p < 0.05

| | Average | Average | T test |
|---|---|---|---|
| miRNA | HC | MS | MS vs HC |
| hsa-miR-30d-5p | 2.39876228 | 5.23212361 | 0.003418484 |
| hsa-miR-7706 | 39.0053994 | 235.443153 | 0.013255044 |
| hsa-miR-1246 | 421.837725 | 1908.75861 | 0.024266179 |
| hsa-miR-148a-3p | 4.63590941 | 1.92817995 | 0.045358167 |
| hsa-miR-770-5p | 0.52302653 | 0 | 0.049701438 |

### Example 4. Oral Administration of Synthetic miR-30d Ameliorates EAE in a Recipient Gut Microbiome-Dependent Manner

To investigate whether miR-30d could affect EAE, we synthesized miR-30d and administered it orally for 7 consecutive days to mice with established EAE. As a control, we administered a scrambled sequence of miR-30d. We found that oral administration of synthetic miR-30d at the dose of 250 pmol, but not its scrambled sequence, ameliorated EAE as measured by clinical score, which was associated with decreased demyelination and axonal loss (FIG. 4A, B). The EAE-improving effect was enhanced with an increasing doses of 1000 pmol and 2500 pmol (FIGS. 8A, B). To address potential cellular mechanisms by which oral miR-30d affected EAE, we examined T cells in the spleen and found an increase in Foxp3+CD4+ Tregs both in the total CD4+ T cell population and in the Vβ11+CD4+ T cell population (FIG. 4C-D, FIG. 9). Vβ11+CD4+ T cell population was MOG-specific (Bettelli et al., 2006a). No change was observed in IFN-γ-expressing Th1 or IL-17-expressing Th17 populations (not shown). We then asked whether the Treg-promoting effect of oral miR-30d was a result from a direct effect of miR-30d on T cell differentiation. We first measured the level of miR-30d in sera of synthetic miR-30d orally treated mice and found no increase of miR-30d in sera (FIG. 10A), suggesting that the orally administered synthetic miR-30d did not enter the circulation. Furthermore, we differentiated naïve CD4+ T cells into Treg, Th1 and Th17 cells in the presence of miR-30d, and found no effect (FIG. 10B). Thus, miR-30d indirectly induces Treg expansion.

The existence of miRNA in the gut lumen and feces has been reported by many studies including ours (Link et al., 2012; Liu et al., 2016; Mohan et al., 2016; Teng et al., 2018; Viennois et al., 2019). MiRNAs are stable (Jung et al., 2010) in a varies of mechanisms including existing in extracellular microvesicle (MV) and/or in a MV-free high-density lipoproteins or argonaute protein-binding form (Creemers et al., 2012). To investigate whether oral delivered miR-30d can survive the gastric acidity and reach intact to the colon. We measured dynamic miR-30d levels in extracellular vesicle fraction and non-vesicle fraction of feces after oral administration of synthetic miR-30d in germ-free mice. We found that synthetic miR-30d reached intact into the colon (feces) in the fraction of 220 nm to 800 nm-sized microvesicle and the fraction of non-vesicle (FIG. 11).

We have previously shown that orally administered miRNAs can shape the microbiome (Liu et al., 2016). To determine whether oral synthetic miR-30d administration induced a protective microbiome phenotype, we orally administered synthetic miR-30d or a scrambled control for 7 consecutive days, starting at the time of immunization. Feces were collected on day 7 post immunization and transferred to naïve recipient mice that had been pre-treated with antibiotics for microbiome depletion and then immunized with MOG/CFA for EAE induction (FIG. 4E). We found that microbiome transferred from miR-30d-treated donor mice ameliorated EAE in recipients as compared to transferred from water-treated or scrambled miR-30d-treated donors (FIG. 4F, G), suggesting that miR-30d treatment enriched microbes that were of regulatory effect for EAE. To further establish that the therapeutic effect of oral miR-30d required the microbiome, we administered antibiotics at the time of oral gavage with synthetic miR-30d and found that antibiotic administration abrogated the protective effect of oral miR-30d on EAE (FIG. 411, I).

### Example 5. MiR-30d Upregulates AMUC_RS06985, a New β-Galactosidase that is Essential for the Growth of A. muciniphila, and Expands A. muciniphila

We next investigated which components of the microbiome in the recipient were involved in the amelioration of EAE by orally administered miR-30d. We and others have previously reported that host fecal miRNA is able to regulate bacterial gene transcription and growth (Liu et al., 2016; Teng et al., 2018). Our data showed above suggest that it was/were microbe(s) that was/were increased by miR-30d or increased in the EAE feces that mediated the EAE-improving effect; and we showed that A. muciniphila was increased in the feces of EAE and MS. Thus, we asked whether miR-30d could regulate A. muciniphila. We blasted the miR-30d sequence against the whole genome sequence of A. muciniphila and found that three genes (Locus tags: AMUC_RS06985, AMUC_RS07700, and AMUC_RS10850) were potential targets of miR-30d (FIG. 5A). We then cultured A. muciniphila in the presence of synthetic miR-30d or its scrambled sequence and found that miR-30d promoted the expression of two of these candidate genes (Locus tag: AMUC_RS06985, AMUC_RS07700) (FIG. 5B). The function of these genes or their protein products has not been reported. Gene bank sequence annotations suggest that these genes encode putative phosphate-binding protein and putative glycosyl hydrolase family protein, respectively, indicating that these genes may be involved in the utilization of glucose (dextrose). We then asked whether A. muciniphila uses glucose for its survival and growth. As previously reported, A. muciniphila grows well on complete brain heart infusion (BHI) agar which contains 0.2% glucose (Derrien et al., 2004). Deprivation of glucose from BHI (BHI without dextrose) resulted in an impaired bacterial growth, suggesting that glucose is essential for A. muciniphila survival. When we replaced glucose with the disaccharide lactose (can be cleaved by β-galactosidase, also called lactase, into glucose and galactose) in culture, A. muciniphila was able to grow. The use of another disaccharide, sucrose, which requires α-galactosidase to cleave into glucose and fructose, did not support the growth of A. muciniphila. Thus, these data suggest that A. muciniphila has β-galactosidase, which converts lactose to glucose. To test this hypothesis, we used another BHI agar without glucose, but contains mucin, which is favored by A. muciniphila (Derrien et al., 2004), and added X-gal to ascertain the β-galactosidase activity. X-gal is an organic substrate for β-galactosidase that is hydrolyzed to the blue-color product 5,5′-dibromo-4,4′-dichloro-indigo (Kiernan, 2007) (FIG. 12). We found that A. muciniphila colonies turned blue, confirming that A. muciniphila has β-galactosidase to hydrolyze mucin. This is consistent with a study in which β-galactosidase was found to be among mucin-degrading enzymes in the A. muciniphila membrane protein fraction (Ottman et al., 2017). To determine whether the protein products of either AMUC_RS06985 or AMUC_RS07700 are the β-galactosidase in A. muciniphila, we aligned the protein sequences of AMUC_RS06985 or AMUC_RS07700 with the protein sequences of different β-galactosidases. We found that AMUC_RS06985 was homologous to β-galactosidases of several species including Ktedonobacter racemifer (FIG. 5C), Bifidobacterium bifidum, Pectobacterium parmentieri, Streptomyces pratensis, and Rattus norvegicus (not shown). We did not find homology between AMUC_RS07700 and any β-galactosidases. To verify the β-galactosidase activity of AMUC_RS06985, we cloned AMUC_RS06985 into a β-galactosidase-deficient Escherichia coli (E. coli) strain (lacZ4M15) and tested the β-galactosidase activity on an agar containing X-gal. We found that introduction of AMUC_RS06985, but not a truncated AMUC_RS06985, conferred β-galactosidase activity in the β-galactosidase-deficient E. coli strain (FIG. 5D). These data identified AMUC_RS06985 as a new β-galactosidase in A. muciniphila. We and others have showed that microRNA can enter bacteria, regulate the gene expression and growth of bacteria (Liu et al., 2016; Teng et al., 2018). We confirmed through in situ hybridization and transmission electron microscopy that miR-30d was able to enter A. muciniphila (FIG. 13A). We observed that in an in vitro co-culture of A. muciniphila and E. coli, supplement of miR-30d in the culture specifically increased A. muciniphila, as indicated by the increased ratio of A. muciniphila to E. coli (FIG. 13B). We next asked whether miR-30d affected β-galactosidase in A. muciniphila. We cultured A. muciniphila on agar containing lactose and X-gal, and treated them with miR-30d. We found that miR-30d-treated A. muciniphila exhibited a significant enhanced β-galactosidase activity, compared to scrambled miR-30d-treated A. muciniphila (FIG. 5E). Thus, miR-30d can promote A. muciniphila growth by enhancing bacterial β-galactosidase.

We then asked whether oral administration of synthetic miR-30d could affect the abundance of A. muciniphila in mouse gut. We orally gavaged mice with synthetic miR-30d for 7 days starting at the time of MOG-immunization and then analyzed the fecal microbiome by 16S sequencing (FIG. 5F). We found oral administration of synthetic miR-30d did not change the overall microbial structure, as suggested by weighted UniFrac beta-diversity metric assessment (FIG. 5G). However, we found an increase of A. muciniphila in miR-30d treated mice as compared to animals given a scrambled sequence or vehicle (FIG. 5H and Table 4). We confirmed this result by qPCR (FIG. 5I). Thus, it appears that oral miR-30d administration acts by expanding A. muciniphila.

TABLE 4
Gut microbiome modulated by oral miR-30d administration

| | | | | | T test miR- |
|---|---|---|---|---|---|
| | Average | Average | Average | T test miR- | 30d vs |
| Taxonomy | H2O | Scramble | miR-30d | 30d vs H2O | Scramble |
| A. | 40.62% | 39.67% | 38.13% | 0.400574865 | 0.256136688 |
| B. | 6.84% | 8.37% | 11.17% | 0.019933395 | 0.044359774 |
| C. | 6.66% | 7.44% | 6.44% | 0.85029761 | 0.419907036 |
| D. | 6.57% | 6.91% | 5.70% | 0.299359767 | 0.164802645 |
| E. | 7.27% | 4.62% | 5.74% | 0.36942399 | 0.227141707 |
| F. | 5.81% | 5.99% | 4.76% | 0.468354248 | 0.240212088 |
| G. | 3.78% | 4.35% | 3.66% | 0.865748495 | 0.277633604 |
| H. | 3.32% | 2.96% | 3.17% | 0.779467933 | 0.705073469 |
| I. | 2.93% | 2.72% | 1.90% | 0.071269836 | 0.056900803 |
| J. | 2.09% | 2.56% | 1.53% | 0.156442694 | 0.014675399 |
| K. | 0.80% | 0.59% | 3.90% | 0.01289194 | 0.00873431 |
| L. | 1.63% | 1.75% | 1.82% | 0.395879477 | 0.732234042 |
| M. | 11.67% | 12.06% | 12.07% | 0.711915689 | 0.986272997 |

Taxonomy for Table 4:
A. k_Bacteria; p_Bacteroidetes; c_Bacteroidia; o_Bacteroidales; f_S24-7; g_; s_
B. k_Bacteria; p_Bacteroidetes; c_Bacteroidia; o_Bacteroidales; f_Rikenellaceae; g_; s_
C. k_Bacteria; p_Firmicutes; c_Clostridia; o_Clostridiales; f_Clostridiaceae; g_; s_
D. k_Bacteria; p_Firmicutes; c_Clostridia; o_Clostridiales; f_; g_; s_
E. k_Bacteria; p_Firmicutes; c_Bacilli; o_Lactobacillales; f_Lactobacillaceae; g_Lactobacillus; s_
F. k_Bacteria; p_Firmicutes; c_Clostridia; o_Clostridiales; f_Lachnospiraceae; g_; s_
G. k_Bacteria; p_Firmicutes; c_Bacilli; o_Turicibacterales; f_Turicibacteraceae; g_Turicibacter; s_
H. k_Bacteria; p_Bacteroidetes; c_Bacteroidia; o_Bacteroidales; f_Bacteroidaceae; g_Bacteroides
I. k_Bacteria; p_Firmicutes; c_Clostridia; o_Clostridiales; f_Ruminococcaceae; g_Oscillospira; s_
J. k_Bacteria; p_Firmicutes; c_Clostridia; o_Clostridiales; f_Lachnospiraceae
K. k_Bacteria; p_Verrucomicrobia; c_Verrucomicrobiae; o_Verrucomicrobiales; f_Verrucomicrobiaceae; g_Akkermansia; s_muciniphila
L. k_Bacteria; p_Firmicutes; c_Clostridia; o_Clostridiales; f_Ruminococcaceae; g_Ruminococcus; s_
M. Other

### Example 6. A. muciniphila Ameliorates EAE by Stimulating Treg-Driving Cytokines in Dendritic Cells

To directly investigate whether there was an ameliorating effect of A. muciniphila on EAE, we orally treated established EAE with A. muciniphila for 7 consecutive days. We observed a decrease in disease score associated with reduced demyelination and axonal loss (FIG. 6A, B). As observed with synthetic oral miR30d, A. muciniphila treatment also increased Foxp3+ Tregs in the spleen (FIG. 6C, D).

To explore the potential mechanism by which A. muciniphila induces Foxp3+ Tregs, we first investigated whether A. muciniphila had a direct effect on Treg cell differentiation by co-culturing inactivated A. muciniphila or E. coli (as a control) with naïve CD4+ T cells. We found that both bacteria minimally induced Foxp3+ Tregs (FIG. 6E), suggesting that A. muciniphila does not directly induce more Tregs as compared to E. coli. DCs from mesenteric lymph node (MLN) are known to play a crucial role in Treg induction (Coombes et al., 2007; Cording et al., 2014; Pezoldt et al., 2018), we isolated DCs from MLN, pulsed them with inactivated A. muciniphila or E. coli, and co-cultured them with naïve CD4+ T cells. We found that A. muciniphila-pulsed DCs induced significantly more Foxp3+ Tregs as compared to E. coli-treated DCs (FIG. 6F). Thus, DCs are important for the induction of Foxp3+ Tregs by A. muciniphila. We next investigated how A. muciniphila increased the ability of DCs to induce Foxp3+ Tregs. We measured mRNAs of cytokines involved in CD4+ T cell differentiation, including Tgfb, il6 and il1b. We found that both A. muciniphila and E. coli induced Tgfb expression to a similar extent; however, the expression of Il6 and Il1b, known to inhibit Foxp3+ Tregs generation (Bettelli et al., 2006b; Lee et al., 2012; Sutton et al., 2006), was significantly lower in A. muciniphila-stimulated DCs as compared to E. coli-stimulated DCs (FIG. 6G), suggesting that A. muciniphila preferentially stimulates Treg-inducing cytokines. Taken together, these data suggest that miR-30d promotes A. muciniphila growth in the gut that in turn induce Tregs in a mechanism dependent on increased TGF-β and decreased IL-6 and IL-1β production by DCs.

### Example 7. Effect of Administration of miR-7706 and miR-1246 on EAE

We asked that whether two other miRNAs identified in MS stool, miR-7706 and miR-1246, can affect EAE. We synthesized the mimics of miR-7706 and miR-1246 and orally gave to MOG-immunized mice at disease onset when clinically scored 1 at the dose of 250 pmol for 7 consecutive days. We found that both miR-7706 and miR-1246 ameliorated EAE, as indicated by the EAE clinical scores (FIG. 14A) and pathology (FIG. 14B). Thus miR-7706 and miR-1246 are two additional miRNAs that are able to improve disease.

### Example 8. Effect of Administration of miR-30 in Animal Models of MS, T1D, and Obesity

So far there is no treatment for progressive MS. We investigated whether miR-30d could treat progressive MS using animal model. We used the NOD/ShiLtJ (commonly called NOD) mice and immunized them with MOG/CFA for progressive EAE. We treated the mice with 250 pmol miR-30d daily for 14 consecutive days starting when the EAE score=2. We found that miR-30d orally treatment significantly improved the disease (FIG. 15).

We next asked whether synthetic miRNA could treat diseases other than EAE/MS. Type 1 diabetes (T1D) is an autoimmune disease pathologically featured by lower insulin due to loss of pancreatic islets. The NOD/ShiLtJ (commonly called NOD) mice is a polygenic model for autoimmune type 1 diabetes. NOD mouse is characterized by hyperglycemia and insulitis. Dramatic pancreatic insulin decrease occurs in females at about 12 weeks of age. To test the effect of miRNA on T1D, we orally gavaged miR-30d to NOD mice starting prior to onset of disease at 8 weeks of age at the dose of 250 pmol for 11 consecutive days. We found that 11 days orally administration of synthetic miR-30d delayed the disease by 5 weeks (FIG. 16). Our data suggest that the miRNAs we identified from the stool of peak EAE and untreated MS patient stool not only have beneficial effects on EAE, but also have favorable potentials against other autoimmune diseases, such as T1D.

As noted above, miR-30d can modulate gut microbiome; obesity is a condition that has been reported to be associated with perturbations in the microbiome. We investigated whether miR-30d oral treatment can change obesity. We obtained high fat diet (HFD) induced diabetes mouse model (C57BL/J DIO stock No: 380050; Black 6 DIO, the Jackson Laboratory) and kept them on HFD. We treated the DIO mice by oral gavage synthesized Mission® miR-30d or scrambled control at the dose of 500 pmol every other day for 8 weeks starting at 12 weeks of age. IPGTT test was carried out by the end of treatment and lipids in sera were measured. We found that miR-30d treatment significantly improved the glucose tolerance (FIG. 17A), as well as reduced the cholesterol and triglycerides in DIO mice (FIG. 17B), suggesting that miR-30d may be a treatment in lowering lipids in obesity and in improving obesity-related conditions.

### REFERENCES

An, D., Oh, S. F., Olszak, T., Neves, J. F., Avci, F. Y., Erturk-Hasdemir, D., Lu, X., Zeissig, S., Blumberg, R. S., Kasper, D. L., 2014. Sphingolipids from a Symbiotic Microbe Regulate Homeostasis of Host Intestinal Natural Killer T Cells. Cell 156, 123-133. doi:10.1016/j.cell.2013.11.042
Atarashi, K., Tanoue, T., Shima, T., Imaoka, A., Kuwahara, T., Momose, Y., Cheng, G., Yamasaki, S., Saito, T., Ohba, Y., Taniguchi, T., Takeda, K., Hori, S., Ivanov, I. I., Umesaki, Y., Itoh, K., Honda, K., 2011. Induction of colonic regulatory T cells by indigenous Clostridium species. Science (New York, N.Y.) 331, 337-341. doi:10.1126/science.1198469
Baecher-Allan, C., Kaskow, B. J., Weiner, H. L., 2018. Multiple Sclerosis: Mechanisms and Immunotherapy. Neuron 97, 742-768. doi:10.1016/j.neuron.2018.01.021
Barcena, C., Valdes-Mas, R., Mayoral, P., Garabaya, C., Durand, S., Rodriguez, F., Fernandez-Garcia, M. T., Salazar, N., Nogacka, A. M., Garatachea, N., Bossut, N., Aprahamian, F., Lucia, A., Kroemer, G., Freije, J. M. P., Quiros, P. M., Lopez-Otin, C., 2019. Healthspan and lifespan extension by fecal microbiota transplantation into progeroid mice. Nat Med 25, 1234-1242. doi:10.1038/s41591-019-0504-5
Belkaid, Y., Hand, T. W., 2014. Role of the Microbiota in Immunity and Inflammation. Cell 157, 121-141. doi:10.1016/j.cell.2014.03.011
Benjamin, J. L., Sumpter, R., Levine, B., Hooper, L. V., 2013. Intestinal Epithelial Autophagy Is Essential for Host Defense against Invasive Bacteria. Cell Host Microbe 13, 723-734. doi:10.1016/j.chom.2013.05.004
Berer, K., Gerdes, L. A., Cekanaviciute, E., Jia, X., Xiao, L., Xia, Z., Liu, C., Klotz, L., Stauffer, U., Baranzini, S. E., Kümpfel, T., Hohlfeld, R., Krishnamoorthy, G., Wekerle, H., 2017. Gut microbiota from multiple sclerosis patients enables spontaneous autoimmune encephalomyelitis in mice. Proceedings of the National Academy of Sciences 114, 10719-10724. doi:10.1073/pnas.1711233114
Berer, K., Mues, M., Koutrolos, M., Rasbi, Z. A., Boziki, M., Johner, C., Wekerle, H., Krishnamoorthy, G., 2011. Commensal microbiota and myelin autoantigen cooperate to trigger autoimmune demyelination. Nature 479, 538-541. doi:10.1038/nature10554
Bettelli, E., Baeten, D., Jager, A., Sobel, R. A., Kuchroo, V. K., 2006a. Myelin oligodendrocyte glycoprotein-specific T and B cells cooperate to induce a Devic-like disease in mice. The Journal of Clinical Investigation 116, 2393-2402. doi:10.1172/JCI28334
Bettelli, E., Carrier, Y., Gao, W., Korn, T., Strom, T. B., Oukka, M., Weiner, H. L., Kuchroo, V. K., 2006b. Reciprocal developmental pathways for the generation of pathogenic effector TH17 and regulatory T cells. Nature 441, 235-238. doi:10.1038/nature04753
Blacher, E., Bashiardes, S., Shapiro, H., Rothschild, D., Mor, U., Dori-Bachash, M., Kleimeyer, C., Moresi, C., Harnik, Y., Zur, M., Zabari, M., Brik, R. B.-Z., Kviatcovsky, D., Zmora, N., Cohen, Y., Bar, N., Levi, I., Amar, N., Mehlman, T., Brandis, A., Biton, I., Kuperman, Y., Tsoory, M., Alfahel, L., Harmelin, A., Schwartz, M., Israelson, A., Arike, L., Johansson, M. E. V., Hansson, G. C., Gotkine, M., Segal, E., Elinav, E., 2019. Potential roles of gut microbiome and metabolites in modulating ALS in mice. Nature. doi:10.1038/s41586-019-1443-5
Caporaso, J. G., Kuczynski, J., Stombaugh, J., Bittinger, K., Bushman, F. D., Costello, E. K., Fierer, N., Pena, A. G., Goodrich, J. K., Gordon, J. I., Huttley, G. A., Kelley, S. T., Knights, D., Koenig, J. E., Ley, R. E., Lozupone, C. A., McDonald, D., Muegge, B. D., Pirrung, M., Reeder, J., Sevinsky, J. R., Turnbaugh, P. J., Walters, W. A., Widmann, J., Yatsunenko, T., Zaneveld, J., Knight, R., 2010. QIIME allows analysis of high-throughput community sequencing data. Nat. Methods 7, 335-336. doi:10.1038/nmeth.f.303
Caporaso, J. G., Lauber, C. L., Walters, W. A., Berg-Lyons, D., Huntley, J., Fierer, N., Owens, S. M., Betley, J., Fraser, L., Bauer, M., Gormley, N., Gilbert, J. A., Smith, G., Knight, R., 2012. Ultra-high-throughput microbial community analysis on the Illumina HiSeq and MiSeq platforms. The ISME Journal 6, 1621-1624. doi:10.1038/ismej.2012.8
Cekanaviciute, E., Yoo, B. B., Runia, T. F., Debelius, J. W., Singh, S., Nelson, C. A., Kanner, R., Bencosme, Y., Lee, Y. K., Hauser, S. L., Crabtree-Hartman, E., Sand, I. K., Gacias, M., Zhu, Y., Casaccia, P., Cree, B. A. C., Knight, R., Mazmanian, S. K., Baranzini, S. E., 2017. Gut bacteria from multiple sclerosis patients modulate human T cells and exacerbate symptoms in mouse models. Proceedings of the National Academy of Sciences 114, 10713-10718. doi:10.1073/pnas.1711235114
Chen, J., Chia, N., Kalari, K. R., Yao, J. Z., Novotna, M., Soldan, M. M. P., Luckey, D. H., Marietta, E. V., Jeraldo, P. R., Chen, X., Weinshenker, B. G., Rodriguez, M., Kantarci, O. H., Nelson, H., Murray, J. A., Mangalam, A. K., 2016. Multiple sclerosis patients have a distinct gut microbiota compared to healthy controls. Sci Rep 6, 28484. doi:10.1038/srep28484
Coombes, J. L., Siddiqui, K. R. R., Arancibia-Cárcamo, C. V., Hall, J., Sun, C.-M., Belkaid, Y., Powrie, F., 2007. A functionally specialized population of mucosal CD103+ DCs induces Foxp3+ regulatory T cells via a TGF-beta and retinoic acid-dependent mechanism. J Exp Med 204, 1757-1764. doi:10.1084/jem.20070590
Cording, S., Wahl, B., Kulkarni, D., Chopra, H., Pezoldt, J., Buettner, M., Dummer, A., Hadis, U., Heimesaat, M., Bereswill, S., Falk, C., Bode, U., Hamann, A., Fleissner, D., Huehn, J., Pabst, O., 2014. The intestinal micro-environment imprints stromal cells to promote efficient Treg induction in gut-draining lymph nodes. Mucosal Immunol 7, 359-368. doi:10.1038/mi.2013.54
Creemers, E. E., Tijsen, A. J., Pinto, Y. M., 2012. Circulating microRNAs: novel biomarkers and extracellular communicators in cardiovascular disease? Circ. Res. 110, 483-495. doi:10.1161/CIRCRESAHA.111.247452
Derrien, M., Vaughan, E. E., Plugge, C. M., de Vos, W. M., 2004. Akkermansia muciniphila gen. nov., sp. nov., a human intestinal mucin-degrading bacterium. Int. J. Syst. Evol. Microbiol. 54, 1469-1476. doi:10.1099/ijs.0.02873-0
Everard, A., Belzer, C., Geurts, L., Ouwerkerk, J. P., Druart, C., Bindels, L. B., Guiot, Y., Derrien, M., Muccioli, G. G., Delzenne, N. M., de Vos, W. M., Cani, P. D., 2013. Cross-talk between Akkermansia muciniphila and intestinal epithelium controls diet-induced obesity. Proceedings of the National Academy of Sciences 110, 9066-9071. doi:10.1073/pnas.1219451110
Fung, T. C., Olson, C. A., Hsiao, E. Y., 2017. Interactions between the microbiota, immune and nervous systems in health and disease. Nat Neurosci 20, 145-155. doi:10.1038/nn.4476
Hansen, C. H. F., Krych, L., Nielsen, D. S., Vogensen, F. K., Hansen, L. H., Sorensen, S. J., Buschard, K., Hansen, A. K., 2012. Early life treatment with vancomycin propagates Akkermansia muciniphila and reduces diabetes incidence in the NOD mouse. Diabetologia 55, 2285-2294. doi:10.1007/s00125-012-2564-7
Honda, K., Littman, D. R., 2016. The microbiota in adaptive immune homeostasis and disease. Nature 535, 75-84. doi:10.1038/nature18848
Hooper, L. V., Littman, D. R., Macpherson, A. J., 2012. Interactions between the microbiota and the immune system. Science (New York, N.Y.) 336, 1268-1273. doi:10.1126/science.1223490
Hsiao, E. Y., McBride, S. W., Hsien, S., Sharon, G., Hyde, E. R., McCue, T., Codelli, J. A., Chow, J., Reisman, S. E., Petrosino, J. F., Patterson, P. H., Mazmanian, S. K., 2013. Microbiota modulate behavioral and physiological abnormalities associated with neurodevelopmental disorders. Cell 155, 1451-1463. doi:10.1016/j.cell.2013.11.024
Ianiro, G., Bibbo, S., Scaldaferri, F., Gasbarrini, A., Cammarota, G., 2014. Fecal microbiota transplantation in inflammatory bowel disease: beyond the excitement. Medicine (Baltimore) 93, e97-e97. doi:10.1097/MD.0000000000000097
Ivanov, Atarashi, K., Manel, N., Brodie, E. L., Shima, T., Karaoz, U., Wei, D., Goldfarb, K. C., Santee, C. A., Lynch, S. V., Tanoue, T., Imaoka, A., Itoh, K., Takeda, K., Umesaki, Y., Honda, K., Littman, D. R., 2009. Induction of Intestinal Th17 Cells by Segmented Filamentous Bacteria. Cell 139, 14-14. doi: 10.1016/j.cell.2009.09.033
Jangi, S., Gandhi, R., Cox, L. M., Li, N., Glehn, Von, F., Yan, R., Patel, B., Mazzola, M. A., Liu, S., Glanz, B. L., Cook, S., Tankou, S., Stuart, F., Melo, K., Nejad, P., Smith, K., Topcuolu, B. D., Holden, J., Kivisakk, P., Chitnis, T., De Jager, P. L., Quintana, F. J., Gerber, G. K., Bry, L., Weiner, H. L., 2016. Alterations of the human gut microbiome in multiple sclerosis. Nat Commun 7, 12015. doi:10.1038/ncomms12015
Johnston, D. G. W., Williams, M. A., Thaiss, C. A., Cabrera-Rubio, R., Raverdeau, M., McEntee, C., Cotter, P. D., Elinav, E., O'Neill, L. A. J., Con, S. C., 2018. Loss of MicroRNA-21 Influences the Gut Microbiota, Causing Reduced Susceptibility in a Murine Model of Colitis. J Crohns Colitis 12, 835-848. doi:10.1093/ecco-jcc/jjy038
Jung, M., Schaefer, A., Steiner, I., Kempkensteffen, C., Stephan, C., Erbersdobler, A., Jung, K., 2010. Robust microRNA stability in degraded RNA preparations from human tissue and cell samples. Clin. Chem. 56, 998-1006. doi:10.1373/clinchem.2009.141580
Kiernan, J. A., 2007. Indigogenic substrates for detection and localization of enzymes. Biotech Histochem 82, 73-103. doi:10.1080/10520290701375278
Kopylova, E., Noe, L., Touzet, H., 2012. SortMeRNA: fast and accurate filtering of ribosomal RNAs in metatranscriptomic data. Bioinformatics 28, 3211-3217. doi:10.1093/bioinformatics/bts611
Koutrolos, M., Berer, K., Kawakami, N., Wekerle, H., Krishnamoorthy, G., 2014. Treg cells mediate recovery from EAE by controlling effector T cell proliferation and motility in the CNS. Acta Neuropathol Commun 2, 163. doi:10.1186/s40478-014-0163-1
Krüger, J., Rehmsmeier, M., 2006. RNAhybrid: microRNA target prediction easy, fast and flexible. Nucleic Acids Research 34, W451-4. doi:10.1093/nar/gk1243
Lee, Y., Awasthi, A., Yosef, N., Quintana, F. J., Xiao, S., Peters, A., Wu, C., Kleinewietfeld, M., Kunder, S., Hafler, D. A., Sobel, R. A., Regev, A., Kuchroo, V. K., 2012. Induction and molecular signature of pathogenic TH17 cells. Nat Immunol 13, 991-999. doi:10.1038/ni.2416
Link, A., Balaguer, F., Shen, Y., Nagasaka, T., Lozano, J. J., Boland, C. R., Goel, A., 2010. Fecal MicroRNAs as novel biomarkers for colon cancer screening. Cancer Epidemiol. Biomarkers Prev. 19, 1766-1774. doi:10.1158/1055-9965.EPI-10-0027
Link, A., Becker, V., Goel, A., Wex, T., Malfertheiner, P., 2012. Feasibility of fecal microRNAs as novel biomarkers for pancreatic cancer. PLoS ONE 7, e42933. doi:10.1371/journal.pone.0042933.s001
Liu, S., da Cunha, A. P., Rezende, R. M., Cialic, R., Wei, Z., Bry, L., Comstock, L. E., Gandhi, R., Weiner, H. L., 2016. The Host Shapes the Gut Microbiota via Fecal MicroRNA. Cell Host Microbe 19, 32-43. doi:10.1016/j.chom.2015.12.005
Love, M. I., Huber, W., Anders, S., 2014. Moderated estimation of fold change and dispersion for RNA-seq data with DESeq2. Genome Biol 15, 550. doi:10.1186/s13059-014-0550-8
Lu, L.-F., Rudensky, A., 2009. Molecular orchestration of differentiation and function of regulatory T cells. Genes \& Development 23, 1270-1282. doi:10.1101/gad.1791009
Mayo, L., Trauger, S. A., Blain, M., Nadeau, M., Patel, B., Alvarez, J. I., Mascanfroni, I. D., Yeste, A., Kivisakk, P., Kallas, K., Ellezam, B., Bakshi, R., Prat, A., Antel, J. P., Weiner, H. L., Quintana, F. J., 2014. Regulation of astrocyte activation by glycolipids drives chronic CNS inflammation. Nat Med 20, 1147-1156. doi:10.1038/nm.3681
Mazmanian, S. K., Liu, C. H., Tzianabos, A. O., Kasper, D. L., 2005. An immunomodulatory molecule of symbiotic bacteria directs maturation of the host immune system. Cell 122, 107-118. doi:10.1016/j.cell.2005.05.007
Mohan, M., Chow, C.-E. T., Ryan, C. N., Chan, L. S., Dufour, J., Aye, P. P., Blanchard, J., Moehs, C. P., Sestak, K., 2016. Dietary Gluten-Induced Gut Dysbiosis Is Accompanied by Selective Upregulation of microRNAs with Intestinal Tight Junction and Bacteria-Binding Motifs in Rhesus Macaque Model of Celiac Disease. Nutrients 8. doi:10.3390/nu8110684
Moreira, T. G., Horta, L. S., Gomes-Santos, A. C., Oliveira, R. P., Queiroz, N. M. G. P., Mangani, D., Daniel, B., Vieira, A. T., Liu, S., Rodrigues, A. M., Gomes, D. A., Gabriely, G., Ferreira, E., Weiner, H. L., Rezende, R. M., Nagy, L., Faria, A. M. C., 2019. CLA-supplemented diet accelerates experimental colorectal cancer by inducing TGF-β-producing macrophages and T cells. Mucosal Immunol 12, 188-199. doi:10.1038/s41385-018-0090-8
Nadkarni, M. A., Martin, F. E., Jacques, N. A., Hunter, N., 2002. Determination of bacterial load by real-time PCR using a broad-range (universal) probe and primers set. Microbiology (Reading, Engl.) 148, 257-266. doi: 10.1099/00221287-148-1-257
Nath, N., Khan, M., Paintlia, M. K., Singh, I., Hoda, M. N., Giri, S., 2009. Metformin attenuated the autoimmune disease of the central nervous system in animal models of multiple sclerosis. J Immunol 182, 8005-8014. doi:10.4049/jimmunol.0803563
Norrander, J., Kempe, T., Messing, J., 1983. Construction of improved M13 vectors using oligodeoxynucleotide-directed mutagenesis. Gene 26, 101-106.
Notredame, C., Higgins, D. G., Heringa, J., 2000. T-Coffee: A novel method for fast and accurate multiple sequence alignment. J. Mol. Biol. 302, 205-217. doi:10.1006/jmbi.2000.4042
Ochoa-Repáraz, J., Mielcarz, D. W., Ditrio, L. E., Burroughs, A. R., Foureau, D. M., Haque-Begum, S., Kasper, L. H., 2009. Role of gut commensal microflora in the development of experimental autoimmune encephalomyelitis. J Immunol 183, 6041-6050. doi:10.4049/jimmunol.0900747
Olson, C. A., Vuong, H. E., Yano, J. M., Liang, Q. Y., Nusbaum, D. J., Hsiao, E. Y., 2018. The Gut Microbiota Mediates the Anti-Seizure Effects of the Ketogenic Diet. Cell 173, 1728-1741.e13. doi:10.1016/j.cell.2018.04.027
Ott, S. J., Musfeldt, M., Wenderoth, D. F., Hampe, J., Brant, O., Fölsch, U. R., Timmis, K. N., Schreiber, S., 2004. Reduction in diversity of the colonic mucosa associated bacterial microflora in patients with active inflammatory bowel disease. Gut 53, 685-693. doi:10.1136/gut.2003.025403
Ott, S. J., Waetzig, G. H., Rehman, A., Moltzau-Anderson, J., Bharti, R., Grasis, J. A., Cassidy, L., Tholey, A., Fickenscher, H., Seegert, D., Rosenstiel, P., Schreiber, S., 2017. Efficacy of Sterile Fecal Filtrate Transfer for Treating Patients With Clostridium difficile Infection. Gastroenterology 152, 799-811.e7. doi:10.1053/j.gastro.2016.11.010
Ottman, N., Reunanen, J., Meijerink, M., Pietila, T. E., Kainulainen, V., Klievink, J., Huuskonen, L., Aalvink, S., Skurnik, M., Boeren, S., Satokari, R., Mercenier, A., Palva, A., Smidt, H., de Vos, W. M., Belzer, C., 2017. Pili-like proteins of Akkermansia muciniphila modulate host immune responses and gut barrier function. PLoS ONE 12, e0173004. doi:10.1371/journal.pone.0173004
Pezoldt, J., Pasztoi, M., Zou, M., Wiechers, C., Beckstette, M., Thierry, G. R., Vafadarnejad, E., Floess, S., Arampatzi, P., Buettner, M., Schweer, J., Fleissner, D., Vital, M., Pieper, D. H., Basic, M., Dersch, P., Strowig, T., Hornef, M., Bleich, A., Bode, U., Pabst, O., Bajenoff, M., Saliba, A.-E., Huehn, J., 2018. Neonatally imprinted stromal cell subsets induce tolerogenic dendritic cells in mesenteric lymph nodes. Nat Commun 9, 3903. doi:10.1038/s41467-018-06423-7
Plovier, H., Everard, A., Druart, C., Depommier, C., Van Hul, M., Geurts, L., Chilloux, J., Ottman, N., Duparc, T., Lichtenstein, L., Myridakis, A., Delzenne, N. M., Klievink, J., Bhattacharjee, A., van der Ark, K. C. H., Aalvink, S., Martinez, L. O., Dumas, M.-E., Maiter, D., Loumaye, A., Hermans, M. P., Thissen, J.-P., Belzer, C., de Vos, W. M., Cani, P. D., 2017. A purified membrane protein from Akkermansia muciniphila or the pasteurized bacterium improves metabolism in obese and diabetic mice. Nat Med 23, 107-113. doi:10.1038/nm.4236
Qin, J., Li, Y., Cai, Z., Li, S., Zhu, J., Zhang, F., Liang, S., Zhang, W., Guan, Y., Shen, D., Peng, Y., Zhang, D., Jie, Z., Wu, W., Qin, Y., Xue, W., Li, J., Han, L., Lu, D., Wu, P., Dai, Y., Sun, X., Li, Z., Tang, A., Zhong, S., Li, X., Chen, W., Xu, R., Wang, M., Feng, Q., Gong, M., Yu, J., Zhang, Y., Zhang, M., Hansen, T., Sanchez, G., Raes, J., Falony, G., Okuda, S., Almeida, M., LeChatelier, E., Renault, P., Pons, N., Batto, J.-M., Zhang, Z., Chen, H., Yang, R., Zheng, W., Li, S., Yang, H., Wang, J., Ehrlich, S. D., Nielsen, R., Pedersen, O., Kristiansen, K., Wang, J., 2012. A metagenome-wide association study of gut microbiota in type 2 diabetes. Nature 490, 55-60. doi:10.1038/nature11450
Rehmsmeier, M., Steffen, P., Hochsmann, M., Giegerich, R., 2004. Fast and effective prediction of microRNA/target duplexes. RNA 10, 1507-1517. doi:10.1261/rna.5248604
Reuter, E., Gollan, R., Grohmann, N., Paterka, M., Salmon, H., Birkenstock, J., Richers, S., Leuenberger, T., Brandt, A. U., Kuhlmann, T., Zipp, F., Siffrin, V., 2015. Cross-recognition of a myelin peptide by CD8+ T cells in the CNS is not sufficient to promote neuronal damage. J. Neurosci. 35, 4837-4850. doi:10.1523/JNEUROSCI.3380-14.2015
Rezende, R. M., da Cunha, A. P., Kuhn, C., Rubino, S., M'Hamdi, H., Gabriely, G., Vandeventer, T., Liu, S., Cialic, R., Pinheiro-Rosa, N., Oliveira, R. P., Gaublomme, J. T., Obholzer, N., Kozubek, J., Pochet, N., Faria, A. M. C., Weiner, H. L., 2015. Identification and characterization of latency-associated peptide-expressing γδ T cells. Nat Commun 6, 8726. doi:10.1038/ncomms9726
Robinson, A. P., Harp, C. T., Noronha, A., Miller, S. D., 2014. The experimental autoimmune encephalomyelitis (EAE) model of MS: utility for understanding disease pathophysiology and treatment. Handb Clin Neurol 122, 173-189. doi: 10.1016/B978-0-444-52001-2.00008-X
Round, J. L., Lee, S. M., Li, J., Tran, G., Jabri, B., Chatila, T. A., Mazmanian, S. K., 2011. The Toll-like receptor 2 pathway establishes colonization by a commensal of the human microbiota. Science (New York, N.Y.) 332, 974-977. doi:10.1126/science.1206095
Schmidt, T. S. B., Raes, J., Bork, P., 2018. The Human Gut Microbiome: From Association to Modulation. Cell 172, 1198-1215. doi:10.1016/j.cell.2018.02.044
Schmittgen, T. D., Livak, K. J., 2008. Analyzing real-time PCR data by the comparative C(T) method. Nat Protoc 3, 1101-1108. doi:10.1038/nprot.2008.73
Schönauen, K., Le, N., Arnim, von, U., Schulz, C., Malfertheiner, P., Link, A., 2018. Circulating and Fecal microRNAs as Biomarkers for Inflammatory Bowel Diseases. Inflammatory Bowel Diseases 142, 46. doi:10.1093/ibd/izy046
Sefik, E., Geva-Zatorsky, N., Oh, S., Konnikova, L., Zemmour, D., McGuire, A. M., Burzyn, D., Ortiz-Lopez, A., Lobera, M., Yang, J., Ghosh, S., Earl, A., Snapper, S. B., Jupp, R., Kasper, D., Mathis, D., Benoist, C., 2015. Individual intestinal symbionts induce a distinct population of RORγ+ regulatory T cells. Science (New York, N.Y.) 349, 993-997. doi:10.1126/science.aaa9420
Shin, N.-R., Lee, J.-C., Lee, H.-Y., Kim, M.-S., Whon, T. W., Lee, M.-S., Bae, J.-W., 2014. An increase in the Akkermansia spp. population induced by metformin treatment improves glucose homeostasis in diet-induced obese mice. Gut 63, 727-735. doi:10.1136/gutjnl-2012-303839
Subramanian, S. L., Kitchen, R. R., Alexander, R., Carter, B. S., Cheung, K.-H., Laurent, L. C., Pico, A., Roberts, L. R., Roth, M. E., Rozowsky, J. S., Su, A. I., Gerstein, M. B., Milosavljevic, A., 2015. Integration of extracellular RNA profiling data using metadata, biomedical ontologies and Linked Data technologies. J Extracell Vesicles 4, 27497. doi:10.3402/jev.v4.27497
Sutton, C., Brereton, C., Keogh, B., Mills, K. H. G., Lavelle, E. C., 2006. A crucial role for interleukin (IL)-1 in the induction of IL-17-producing T cells that mediate autoimmune encephalomyelitis. J Exp Med 203, 1685-1691. doi: 10.1084/j em.20060285
Tankou, S. K., Regev, K., Healy, B. C., Tjon, E., Laghi, L., Cox, L. M., Kivisakk, P., Pierre, I. V., Hrishikesh, L., Gandhi, R., Cook, S., Glanz, B., Stankiewicz, J., Weiner, H. L., 2018. A probiotic modulates the microbiome and immunity in multiple sclerosis. Annals of Neurology 83, 1147-1161. doi:10.1002/ana.25244
Teng, Y., Ren, Y., Sayed, M., Hu, X., Lei, C., Kumar, A., Hutchins, E., Mu, J., Deng, Z., Luo, C., Sundaram, K., Sriwastva, M. K., Zhang, L., Hsieh, M., Reiman, R., Haribabu, B., Yan, J., Jala, V. R., Miller, D. M., Van Keuren-Jensen, K., Merchant, M. L., McClain, C. J., Park, J. W., Egilmez, N. K., Zhang, H.-G., 2018. Plant-Derived Exosomal MicroRNAs Shape the Gut Microbiota. Cell Host Microbe 24, 637-652.e8. doi:10.1016/j.chom.2018.10.001
Tremaroli, V., Backhed, F., 2012. Functional interactions between the gut microbiota and host metabolism. Nature 489, 242-249. doi:10.1038/nature11552
Tremlett, H., Fadrosh, D. W., Faruqi, A. A., Zhu, F., Hart, J., Roalstad, S., Graves, J., Lynch, S., Waubant, E., US Network of Pediatric MS Centers, 2016. Gut microbiota in early pediatric multiple sclerosis: a case-control study. Eur. J. Neurol. 23, 1308-1321. doi:10.1111/ene.13026
Turnbaugh, P. J., Hamady, M., Yatsunenko, T., Cantarel, B. L., Duncan, A., Ley, R. E., Sogin, M. L., Jones, W. J., Roe, B. A., Affourtit, J. P., Egholm, M., Henrissat, B., Heath, A. C., Knight, R., Gordon, J. I., 2008. A core gut microbiome in obese and lean twins. Nature 457, 480-484. doi:10.1038/nature07540
van Nood, E., Vrieze, A., Nieuwdorp, M., Fuentes, S., Zoetendal, E. G., de Vos, W. M., Visser, C. E., Kuijper, E. J., Bartelsman, J. F. W. M., Tijssen, J. G. P., Speelman, P., Dijkgraaf, M. G. W., Keller, J. J., 2013. Duodenal infusion of donor feces for recurrent Clostridium difficile. N. Engl. J. Med. 368, 407-415. doi:10.1056/NEJMoa1205037
Viennois, E., Chassaing, B., Tahsin, A., Pujada, A., Wang, L., Gewirtz, A. T., Merlin, D., 2019. Host-derived fecal microRNAs can indicate gut microbiota healthiness and ability to induce inflammation. Theranostics 9, 4542-4557. doi:10.7150/thno.35282
Waterhouse, A. M., Procter, J. B., Martin, D. M. A., Clamp, M., Barton, G. J., 2009. Jalview Version 2—a multiple sequence alignment editor and analysis workbench. Bioinformatics 25, 1189-1191. doi:10.1093/bioinformatics/btp033
Wei, Z., Batagov, A. O., Schinelli, S., Wang, J., Wang, Y., Fatimy, El, R., Rabinovsky, R., Balaj, L., Chen, C. C., Hochberg, F., Carter, B., Breakefield, X. O., Krichevsky, A. M., 2017. Coding and noncoding landscape of extracellular RNA released by human glioma stem cells. Nat Commun 8, 1145-15. doi:10.1038/s41467-017-01196-x
Wu, H., Esteve, E., Tremaroli, V., Khan, M. T., Caesar, R., Mannerås-Holm, L., Ståhlman, M., Olsson, L. M., Serino, M., Planas-Félix, M., Xifra, G., Mercader, J. M., Torrents, D., Burcelin, R., Ricart, W., Perkins, R., Fernandez-Real, J. M., Bäckhed, F., 2017. Metformin alters the gut microbiome of individuals with treatment-naive type 2 diabetes, contributing to the therapeutic effects of the drug. Nat Med 23, 850-858. doi:10.1038/nm.4345
Yissachar, N., Zhou, Y., Ung, L., Lai, N. Y., Mohan, J. F., Ehrlicher, A., Weitz, D. A., Kasper, D. L., Chiu, I. M., Mathis, D., Benoist, C., 2017. An Intestinal Organ Culture System Uncovers a Role for the Nervous System in Microbe-Immune Crosstalk. Cell 168, 1135-1148.e12. doi:10.1016/j.cell.2017.02.009

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating, reducing risk of development or progression of, or reducing symptoms of, an inflammatory condition in a subject, the method comprising administering a therapeutically effective amount of a nucleic acid comprising a sequence that is identical to a contiguous sequence of at least 12 nucleotides present in mature miR-30d, miR-7706, and/or miR-1246 microRNA, to a subject in need thereof.

2. A method of reducing interferon gamma (IFNγ)-producing Th1 and/or interleukin-17 (IL-17)-secreting Th17 CD4+ T cells, and/or increasing regulatory cells such as FoxP3+ regulatory T cells (Tregs), in the periphery and/or in the CNS in a subject, the method comprising administering a therapeutically effective amount of a nucleic acid comprising a sequence that is identical to a contiguous sequence of at least 12 nucleotides present in mature miR-30d, miR-7706, and/or miR-1246 microRNA, to a subject in need thereof.

3. The method of claim 1, wherein the nucleic acid is a miRNA selected from miR-30d, miR-7706, and/or miR-1246.

4. The method of claim 1, comprising administering miR-30d; miR-7706; miR-1246; miR-30d and miR-7706; miR-30d and miR-1246; miR-7706 and miR-1246; or miR-30d, miR-7706, and miR-1246.

5. A method of increasing relative abundance of Akkermansia muciniphila in the gut microbiome of a subject, the method comprising administering a therapeutically effective amount of a nucleic acid comprising a sequence that is identical to a contiguous sequence of at least 12 nucleotides present in mature miR-30d to a subject in need thereof.

6. The method of claim 2, wherein the subject has an inflammatory condition.

7. The method of claim 1, wherein the condition is an inflammatory autoimmune disease.

8. The method of claim 1, wherein the condition is selected from the group consisting of Type 1 diabetes; multiple sclerosis; inflammatory bowel disease (IBD)/colitis; obesity and obesity-related conditions; epilepsy; immune-mediated liver injury; amyotrophic lateral sclerosis (ALS); rheumatoid arthritis; and aging or progeria.

9. The method of claim 1, wherein the nucleic acid is a miRNA mimic.

10. The method of claim 9, wherein the miRNA mimic comprises one or more modifications.

11. The method of claim 10, wherein the modifications include but are not limited to: double-stranded sequence, 5′ Amino-Modifier C6, and/or 3′ [dT][dT].

12. The method of claim 1, wherein the nucleic acid is administered orally.

13. The method of claim 1, wherein the nucleic acid is administered rectally.

14-26. (canceled)
